(19) United States
(12) Patent Application Publication
(10) Pub. No.: US 2007/0142317 A1
(43) Pub. Date: Jun. 21, 2007

(54) Topical composition for treatment of skin disorders

(75) Inventors: Stephen L. Warren, Westfield, CO (US); Jerome Arthur Morris, Fort Collins, CO (US); Cody L. Yarborough, Cleburne, TX (US)

Correspondence Address: SCHWEGMAN, LUNDBERG, WOESSNER & KLUTH, P.A., P.O. BOX 2938, MINNEAPOLIS, MN, 55402, US

(73) Assignee: QLT USA, Inc., Fort Collins, CO (US)

(21) Appl. No.: 11/478,362

(22) Filed: Jun. 29, 2006

Related U.S. Application Data
(63) Continuation of application No. PCT/US04/44060, filed on Dec. 29, 2004.
(60) Provisional application No. 60/599,445, filed on Aug. 6, 2004.
(60) Provisional application No. 60/533,647, filed on Dec. 29, 2003.
(60) Provisional application No. 60/578,165, filed on Jun. 9, 2004.

(51) Int. Cl. A61K 31/7076

(52) U.S. Cl. 514/46

(57) ABSTRACT

The present invention provides for a topical composition that includes a topical carrier and an adenosine deaminase inhibitor. Suitable specific adenosine deaminase inhibitors include, e.g., deoxycoformycin (dCF), deoxyadenosine (dAdo), cladrabine (CdA), fludarabine (F-Ara-A), cytrabine (Ara-C), and thioguanine. The present invention also provides for a method to treat lymphocyte mediated skin diseases and to alleviate symptoms associated with such skin diseases. The method includes topically administering the composition to a mammal in need of such treatment. The present invention also provides for kits and syringe systems that include the adenosine deaminase inhibitor.

### RELATED APPLICATIONS

This invention claims priority to U.S. Ser. No. 60/599,445 filed on 6 Aug. 2004; to U.S. Ser. No. 60/533,647 filed on 29 Dec. 2003; and to U.S. Ser. No. 60/578,165 filed on Jun. 9, 2004; all of which are incorporated by reference herein, in their entirety.

### BACKGROUND OF THE INVENTION

Lymphocyte-mediated chronic skin disorders are a broad group of skin diseases that are driven by an immunological response involving a sub-class of white blood cells called lymphocytes. Typically, lymphocytes aid in the protection against infection and disease; however in numerous immune-mediated chronic skin disorders, lymphocytes may become constitutively or intermittently activated by a variety of pathological mechanisms.

In one class of lymphocyte-mediated skin disease (e.g., psoriasis and alopecia areata) the inflammatory cascade is driven predominantly by T_{H}1 or Tc1 effector cells, which include CD4+ “helper” T-lymphocytes and CD8+ “cytotoxic” T-lymphocytes. In T_{H}1 type inflammation, CD4+ and CD8+ T-cell subsets produce type 1 cytokines, which include interferon-γ (IF-γ), interleukin-2 (IL-2) and tumor necrosis factor-α (TNF-α), but little or no production of interleukins 4, 10 and 11. This maturation pathway is stimulated by interleukin-12 (IL-12), which is released from antigen presenting (dendritic) cells that have been activated by type 1 cytokines. In certain T_{H}1 or Tc1 diseases, such as psoriasis, cytokines produced by dermal and epidermal lymphocytes stimulate keratinocytes in the epidermis to proliferate and differentiate, to express multiple cell adhesion and co-stimulatory molecules (e.g., ICAM, CD40, and MHC type II antigens), and to secrete several chemokines (e.g., IL-8, MIG, IP-10, MIP-3α and RANTES) that intensify and augment the inflammatory cascade.

An example of this class of lymphocyte-mediated skin disease is psoriasis. In its typical form, psoriasis results in plaques of inflamed skin covered with scales. Patients typically experience pain and itching (which can interfere with basic daily functions) and they may also experience social isolation and psychological distress. Psoriasis is one the most prevalent of these skin disorders and affects 6 to 8 million people in the United States. Psoriatic plaque most often occurs on the elbows, knees, other parts of the legs, scalp, lower back, face, palms, and soles of the feet, but it can occur on skin anywhere on the body.

In another class of lymphocyte-mediated skin disease (e.g., atopic dermatitis, lupus erythematosis, bullous pemphigoid), T_{H}2 or Tc2 effector T-cells are generated. The latter T-cells may also be CD4+ or CD8+, but in this type of inflammation, the effector cells release a different spectrum of cytokines, which includes interleukin-4 (IL-4), interleukin-6 (IL-6), interleukin-10 (IL-10), but not IF-γ. The latter cytokines tend to suppress the actions of type 1 effectors and stimulate production of immunoglobulin synthesis by B-lymphocytes. In certain cases, the immunoglobulins are ‘autoreactive’— i.e. directed against self-antigens present in the skin and other tissues. In other cases, the antibodies may be directed against allergens, e.g., IgE antibodies in atopic dermatitis.

There are a number of treatments currently available to people with immune-mediated chronic skin disorders, such as psoriasis. Topical treatments include corticosteroids, calcipotriene, retinoids, anthralin, coal tar, salicylic acid, photochemotherapy with ultraviolet A (PUVA) and phototherapy with UVB. Many of latter treatments are heavily prescribed for treatment of psoriasis, but only PUVA and UVB are capable of killing the T-lymphocytes that drive the disease process. T-lymphocyte killing is required to provide long term (“remittive”) effects lasting months after the therapy is discontinued. However, none of the topical drug treatments mentioned above lead to efficient T-lymphocyte killing within the plaque; therefore, these therapies are generally associated with relapse or rebound shortly after the therapy is discontinued. The latter therapies are commonly referred to as “suppressive” to distinguish them from the remittive type therapies. Most, if not all, currently available topical therapies treat the symptoms rather than the cause of psoriasis.

A new category of biological product is being introduced to the dermatological practitioner that promises better treatment by selectively inhibiting numerous T-cell functions and cytokines. For example, certain biological agents block T-cell adhesion (e.g., anti-E-selectin, antiCD11a); other agents block T-cell proliferation (e.g., anti-CD25 basiliximab, daclizumab); other agents block T-cell activation (e.g., Alefacept, Efalizumab, CTLA4-IG, Anti-CD2 and anti-CD4); and other agents block proinflammatory cytokines such as TNF-α (e.g., etanercept and infliximab). Most of the currently available biological agents do not induce apoptosis of T-cells in the epidermal layer of the plaque and therefore do not produce durable (remittive) responses. Some of these agents appear to be quite effective and have been particularly useful for patients who have moderate to severe psoriasis. However, biological agents are expensive to the patient, must be used systemically, and must be injected intravenously or subcutaneously. Furthermore, essentially all patients treated with biological agents will relapse and/or rebound after the therapy wears off. Biological agents are generally suppressive, rather than remittive.

To date, there are no remittive topical drug products that act primarily by inducing apoptosis of T cells or antigen presenting cells in the psoriatic plaque. By eliminating the antigen presenting cells, one would then eliminate the activation of T-cells, which would interrupt the psoriatic inflammatory cascade at the initial pathogenic step. Additionally, there are no topical medicinal products that act by increasing the apoptotic threshold for lymphocytes. Very little is known about the effect of drugs on the principle antigen presenting cells of the skin, the Langerhans cells. Therefore, there is a need in the art for a topical drug product capable of eliminating Langerhans cells, or their precursors, as well as the T-cells in the epidermal layer of psoriatic plaque. By eliminating the antigen presenting cells, one would eliminate the activation of T-cells, which would interrupt the psoriatic inflammatory cascade at the initial pathogenic step. Remittive response may be generated by elimination of T-cells or Langerhans cells.

Deoxycoformycin (dCF) is an intravenous oncology drug that is used to treat hairy cell leukemia, chronic lymphocytic leukemia, non-Hodgkin's lymphoma, malignant histiocytosis and other hematolymphoid malignancies (Grever et al., J. Clin. Oncol., 3:196 (1985); Malpeis, Cancer Treatment Symposia, 2:7 (1984); Ho, et al., Blood, 72:1884 (1988); Grever, Blood, 57:406 (1981); Lombardi, J. of Pediatrics, 130(2);330 (1997); Lombardi, Hematology J., 3:118 (2002); Weitzman et al., Med. Pediatr. Oncol., 33:476 (1999)). Intravenous dCF has been used in an experimental setting to treat life-threatening autoimmune diseases (Goodman, Seminars in Oncology, 27:67 (2000); Byrd, Seminars in Oncology, 27:1 (2000); Osuji, Br. J. Hemat., 123:297 (2003); Law, Crit. Care Med., 31:1475 (2003); Pavletic, Brief Comm., 877 (2003); Heald, Seminars in Oncology, 27:3 (2000)), and graft-versus-host disease (GvHD) (Margolis, Seminars in Oncology, 27: 9 (2000)). Intravenous dCF has also been used as an experimental therapy for refractory rheumatoid arthritis (Albert, American College of Rheumatology, (1995). Albert, National Scientific Meeting, October 1995), but this was before the advent of effective biological agents that target TNF-alpha.

Systemic administration of dCF results in side effects including serious toxicities such as lymphopenia (lymphotoxicity (i.e. lymphopenia) is an undesirable drug effect in the oncology setting, because it may put the patient at risk for opportunistic infections (Margolis, Seminars in Oncology, 27:9 (2000); Steis, J. Nat. Cancer Inst., 82:1678 (1991); Trotta, Cancer Res., 41:2189 (1981); Seymour, Leukemia 11:42 (1997); Lee, Br. J. Hemat., 56:107 (1984)). In addition to immunosuppression, dCF treated patients may experience skin rash, as well as serious renal, pulmonary and CNS toxicities. (Nipent® package insert; SuperGen, Inc., Dublin Calif.). However, dCF induces apoptosis of non-malignant T-lymphocytes, monocytes as well as neoplastic cell lines derived from the lymphocytic and monocytic lineages (Smyth, Cancer Chemo. Pharm., 5:93 (1980); Ogawa, Tohoku J. Exp Med., 192:87 (2000); Johnston, Leukemia Res., 16:781 (1992); Pettitt, Br. J. Hemat., 121:692 (2003); Niitsu et al., Blood, 96:1512 (1996)). And it is known that autoreactive T-lymphocytes cause psoriasis and other lymphocyte-mediated skin diseases. Furthermore, it is known that monocytes, as well as monocyte-derived macrophages and dendritic (Langerhans) cells have important roles in many autoimmune diseases, such as psoriasis, atopic dermatitis, pemphigus vulgaris, and lupus dermatitis. Thus, by the above criteria, dCF, and other deaminase inhibitors and nucleoside analogs, seem to have the activities needed to treat certain autoimmune diseases.

Despite the ability of dCF to kill the dysregulated immune cells that cause autoimmune diseases, given dCF's history, employing dCF (or other anti-leukemia drugs) as a potentially useful treatment for non-life threatening, mild, moderate or severe autoimmune skin conditions, such as psoriasis or atopic dermatitis, would be counterintuitive. In fact, it seems counterintuitive to use any anticancer drug to treat non-life threatening autoimmune skin disorders such as psoriasis, especially in cases where the disease activity is mild to moderate. The typical objection is that dCF is an oncology drug, and as such, it is considered to be too toxic for non-oncology indications. An examination of the idea may lead to the conclusion that the therapeutic index will simply be too narrow. Finally, there are potential formulation barriers, including the well known aqueous instability of dCF. Specifically, dCF is highly susceptible to degradation (e.g., hydrolysis). As such, an aqueous formulation that includes dCF will have a relatively short shelf-life.

Another way to target the viability of cells is by interfering with their DNA synthesis process so as to induce apoptosis. Nucleotides, nucleosides, bases and analogs thereof, including by not limited to, classes of compounds known as “antimetabolites” (including, but not limited to, cladribine (CdA), Ara-A, Ara-G, fludarabine (F-Ara-A), nelarabine, clofarabine (CAFdA), Ara-C and gemcitabine) affect cells in this manner. Some of these compounds are utilized in anticancer treatments when given systemically or orally (Fludarabine and Clofarabine) and utilized in topical anti-viral treatment (Ara-A and Acyclovir). These compounds differ enough from normal metabolites to interfere with the synthesis of DNA. Antimetabolites, when in their activated, phosphorylated form, are only subtly different from intracellular nucleotides, such as deoxynucleotides. Some of these compounds can indirectly inhibit DNA synthesis by inhibition of DNA metabolizing enzymes such as ribonucleotide reductase (RNR). Inhibition of RNR reduces the pool of deoxyribonucleotides available for DNA synthesis. In lymphoid cells and monocytes this can trigger apoptosis. In addition, these antimetabolites can directly interfere with DNA synthesis by their incorporation into chromosomal DNA to only be recognized later as imperfections, which then halts the process.

In the case of any such compound previously used to treat solid tumors and hematopoietic malignancies, it is counterintuitive that one could use such drugs as a safe treatment for lymphocyte-mediated autoimmune skin disorders. That is because of the well documented serious, potentially life threatening toxicities (e.g., alopecia, bone marrow toxicity, mucositis and gastroenteritis) associated with the systemic administration of antimetabolites in the oncology setting.

Another way to induce apoptosis is to cause an imbalance in the levels of normal metabolites within susceptible cells such as lymphocytes or monocytes. For example, elevation of deoxyadenosine triphosphate (dATP) in the cytoplasm of lymphocytes and monocytes leads to apoptosis. This may be achieved by exposing cells to deoxyadenosine, a precursor of dATP. Thus, deoxyadenosine (dAd), a normal and ubiquitous metabolite, may also be used as a cytotoxic agent which is selective for lymphocytes and monocytes. The use of a normal and ubiquitous metabolite as a cytotoxic agent is counterintuitive. The use of deoxyadenosine to treat psoriasis is not apparent for the reasons stated above, but also because it is not clear how to specifically expose dysregulated T-cells and/or monocytes to dAd at levels that are sufficient to induce apoptosis. For example, the levels must be sufficiently high to overcome the counteractive effects of ADA enzymes, which are abundant in, and on the surface of, cells.

Thus, there is a need in the art for compositions and methods to effectively and safely (e.g., non-toxic) treat skin disorders, in particular immune-based skin disorders, such as psoriasis.

### SUMMARY OF THE INVENTION

The present invention provides compositions and methods to treat lymphocyte mediated skin disease, such as psoriasis or dermatitis. The compositions described herein have a stable shelf life, are relatively non-toxic when used in the topical formulations described herein (in comparison to systemic administration), penetrate into the epidermis to reach the intradermal immune cells which are believed to drive the inflammation that characterizes psoriasis, and affects immune cells, including T-lymphocytes, B-lymphocytes, natural killer cells, monocytes, macrophages, dendritic and Langerhans cells (antigen presenting cells). Methods of using such compositions topically provide an acceptable therapeutic index.

Local administration of the active compounds described herein (e.g., 2′-halo deoxyadenosines) can be accomplished with a safety margin that is much better than its use as a systemic therapy for the leukemia/lymphoma oncology indications; for serious or life-threatening autoimmune diseases, such as GvHD, Nijmegen chromosome breakage syndrome, granulomatous slack skin disease; and for refractory rheumatoid arthritis. The active compounds described herein (e.g., 2′-halo deoxyadenosines) provided herein increase the safety margin of anti-leukemia drugs by a factor of at least about 1.8-fold and greater, as compared to the safety margin when these active compounds are administered systemically for the treatment of the leukemia/lymphoma indications; for serious or life-threatening autoimmune diseases, such as GvHD, Nijmegen chromosome breakage syndrome, granulomatous slack skin disease; and for refractory rheumatoid arthritis.

The relative selectivity of the active compounds described herein (e.g., 2′-halo deoxyadenosines) as lymphocyte toxins enable the compounds to be a safe and effective treatment for diseases driven by abnormally activated T cells. This includes chronic immune-mediated skin disorders (e.g., psoriasis, alopecia areata, atopic dermatitis, lupus erythematosis and bullous pemphigoid). Since the active compounds described herein, such as cladribine, have found success as lymphocyte toxins when systemically delivered, these agents will also find similar benefit in the dermal regions when topically applied.

The topical compositions described herein include a 2′-halo deoxyadenosine, such as cladribine. The amount of 2′-halo deoxyadenosine employed in the topical composition is significantly lower than the amount present, e.g., in intravenous (i.v.) formulations used to treat cancer. Additionally, when present in a topical composition, as opposed to an intravenous composition, it is believed that only a fraction (e.g., less than about 10%) of the 2′-halo deoxyadenosine is systemically absorbed. As such, the topical compositions described herein possess suitable safety profiles.

When the 2′-halo deoxyadenosine is delivered onto the skin, the formulation characteristics that lead to epidermal penetration will cause the drug to come into contact with activated T cells and antigen presenting cells and their precursors (monocytes) within the epidermal plaque and underlying dermis where apoptotic induction occurs. This selective toxic effect on lymphocytes will lead to an accumulation of deoxyadenosine nucleoside triphosphate (dATP), which, by an as yet incompletely understood mechanism, stimulates caspases and ultimately causes apoptosis of the abnormal lymphocytes, monocytes, and antigen-presenting cells, such as Langerhans cells, which are enriched in the psoriatic lesions. Thus, topically administered 2′-halo deoxyadenosine provides a novel and durable treatment of the immune-mediated skin diseases including, but not limited to, psoriasis.

The 2′-halo deoxyadenosine is processed to maximize storage times and to be uniformly dispersed in a viscous vehicle for topical application. The 2′-halo deoxyadenosine can be placed in contact with the skin for a prolonged duration so as to penetrate the skin and act upon diseased cells within the diseased epidermis and underlying dermis. The rate and duration of release of the 2′-halo deoxyadenosine can be modified with the selection of the excipients making up the composition.

Based upon calculated safety margins, it is possible to formulate a 2′-halo deoxyadenosine into safe and effective topical treatments for lymphocyte-mediated autoimmune skin disorders. Such drugs can be formulated so that they adequately penetrate the epidermal and dermal tissues and achieve the desired effects on immune cells, such as cell cycle arrest and apoptosis. Finally, it is possible to generate an acceptable safety profile by limiting systemic exposure to the antimetabolites through topical formulation strategies while achieving the desired pharmacodynamic effects on the immune cells in the epidermis and dermis. These strategies will help to achieve a safety margin that is acceptable for non-life threatening dermatology indications. The present invention includes single agent and combination therapies to achieve the desired synergistic effects and an adequate safety margin.

The invention also provides formulations that include a 2′-halo deoxyadenosine which inhibits the synthesis of DNA (directly or indirectly) and/or are resistant to deamination with the chemical stability required to meet regulatory guidelines and to allow for the manufacture of a commercial product. For example, cladribine forms degradation products by hydrolysis reactions. In one embodiment, the present invention provides for a two-part formulation, so that the product will be stable. In another embodiment, the present invention provides for a one-part product that exhibits the requisite level of stability by including water below a certain maximum threshold, such that hydrolysis is minimized.

One embodiment of the present invention provides a method for treating a skin disorder in a mammal inflicted with a skin disorder including topically administering in an amount effective to induce cell cycle arrest and/or apoptosis of T-lymphocytes, B-lymphocytes, natural killer cells and/or antigen-presenting cells such as monocytes, macrophages, and dendritic (Langerhans) cells within the superficial epidermis and/or dermis. Preferably, when applied as a topical therapy, effective therapeutic levels of the drug will be achieved only in the epidermis and dermis.

The present invention provides a method of treating a psoriasis in a mammal. The method includes topically administering to a mammal in need of such treatment a pharmaceutical composition that includes:

(a) a compound of formula (I): 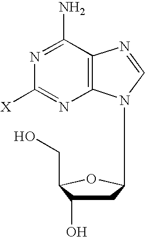
wherein X is F, Cl, Br or I;

(b) a penetration skin enhancer; and

(c) a pharmaceutically acceptable carrier;

in an amount and for a period of time effective to treat the psoriasis.

The present invention also provides the use of a pharmaceutical composition that includes:

(a) a compound of formula (I): 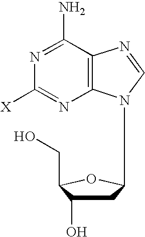
wherein X is F, Cl, Br or I;

(b) a penetration skin enhancer, and

(c) a pharmaceutically acceptable carrier,

for the manufacture of a medicament for treating psoriasis.

The present invention also provides a kit that includes:

(a) a first container comprising a compound of formula (I): 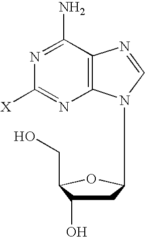
wherein X is F, Cl, Br or I; and

(b) a second container comprising a pharmaceutically acceptable carrier.

The present invention also provides a syringe system that includes:

a first syringe having a female fitting, the first syringe comprising a first syringe barrel having an inner surface and an open proximal end;

a first syringe plunger having a first stopper tip in slidable communication with the inner surface of the first syringe barrel via the open proximal end, the first stopper tip configured for fluid-tight engagement with a first composition;

a second syringe having a male fitting, the second syringe comprising a second syringe barrel having an inner surface and an open proximal end; and

a second syringe plunger having a second stopper tip in slidable communication with the inner surface of the second syringe barrel via the open proximal end, the second stopper tip configured for fluid-tight engagement with a second composition;

wherein the female fitting is sized to receive and configured to interlock with the male fitting for fluid-tight engagement between the first and the second syringes;

the first syringe comprising a compound of formula (I): 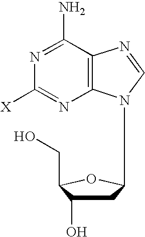
wherein X is F, Cl, Br or I; and the second syringe comprising a pharmaceutically acceptable carrier.

### BRIEF DESCRIPTION OF THE DRAWINGS

FIG. 1 is a perspective view of two syringes in accordance with one embodiment.

FIG. 2 is a side view of a syringe system in accordance with one embodiment.

FIG. 3 is a side view of a syringe system in accordance with one embodiment.

FIG. 4 is a side view of a locking mechanism in accordance with one embodiment.

FIG. 5 is a side view of a syringe system in accordance with one embodiment.

FIG. 6 is a side view of a syringe system in accordance with one embodiment.

FIG. 7 is a schematic of Langerhan and T-cell activation.

### DETAILED DESCRIPTION OF THE INVENTION

As used herein, the following terms and expressions have the indicated meanings.

Definitions

As used herein, “treating” or “treat” includes (i) preventing a pathologic condition (e.g., psoriasis) from occurring; (ii) inhibiting the pathologic condition (e.g., psoriasis) or arresting its development; (iii) relieving the pathologic condition (e.g., psoriasis), or (iv) alleviating the symptoms (e.g., itching) associated with the pathologic condition (e.g., psoriasis).

As used herein, “analog” or “analogue” refers to a chemical with a similar structure to another but differing slightly in composition (as in the replacement of one or more atoms by an atom of a different element (e.g., replacement of an —OH with an H) or in the presence of a particular functional group). For example, a nucleoside analog refers to, for example, an analog of adenosine, including, but not limited to, 2′-deoxyadenosine, 2-flouro-2-deoxyadenosine, 2-chloro-2-deoxyadenosine etc. A nucleoside analog also refers to analogs of guanosine uridine, and cytidine (nucleoside analogs include, but are not limited to, 2-deoxyribose nucleosides, such as 2-deoxyadenosine, 2-deoxyguanosine, 2-deoxycytidine, and 2-deoxythymine and analogs thereof (e.g., 2-chloro-2-deoxyadenosine)). “Analog” or “analogue” also includes a compound that resembles another in structure but is not necessarily an isomer (e.g., 5-fluorouracil is an analog of thymine). Analogs are often used to block enzymatic reactions by combining with enzymes (e.g., isopropyl thiogalactoside vs. Lactose).

An “inhibitor” refers to a substance that restrains or retards physiological, chemical, or enzymatic action to any degree. For example, an enzyme inhibitor is a substance/molecule that restrains or retards enzymatic action. The inhibitor may partially inhibit enzyme activity or fully inhibit enzyme activity. For example, the inhibitor may inhibit (competitively or non-competitively) about 5%, about 10%, about 15%, about 20%, about 30%, about 40%, about 50%, about 60%, about 70%, about 80%, about 90% or about 100% of the natural enzyme activity.

As used herein, “adenosine deaminase inhibitor” refers to a class of compounds that inhibit deamination of nucleosides (many of the inhibitors in this class of compounds interfere with and/or inhibit to some extent the action of an enzyme necessary for the normal synthesis of part of a DNA molecule). Additionally, “adenosine deaminase inhibitors” may possess other biological activities. Many of the compounds in this class are known to be effective for the treatment of hairy cell leukemia, chronic lymphocytic leukemia, Non-Hodgkin's lymphoma, acute lymphocytic leukemia, mycosis fungoides, prolymphocytic leukemia (B-cell and T-cell origin) and T-cell leukemia by selectively inducing apoptosis of lymphocytes. The selectivity of these agents derives partly from the exquisite sensitivity of proliferating lymphocytes to elevated levels of deoxyadenosine triphosphate (dATP), which result from the effective inhibition of adenosine deaminase. Essentially, all non-lymphocytic cells are relatively insensitive to the inhibition of adenosine deaminase, a conclusion strongly supported by the relative lack of pathology in non-lymphoid tissues of patients with a hereditary deficiency of adenosine deaminase. In one specific embodiment of the present invention, the adenosine deaminase inhibitor can be an antimetabolites isolated from, e.g., Streptomyces antiobiotiucus or Aspergillus nidulans. In another specific embodiment of the present invention, the adenosine deaminase inhibitor can be selected from the group of cladribine, deoxycoformycin (pentostatin, Nipent®), coformycin, diethyl pyrocarbonate, erythro-9-(2-hydroxy-3-nonyl) adenine, erythro-9-[3-(2-hydroxynonyl)]adenosine, erythro-9-(2-hydroxy-3-nonyl)-adenosine (EHNA), 6-(R)-hydroxyl-1,6-dihydropurine ribonucleoside (HDPR), imidazole-4-carboxamide derivatives, erythro-6-amino-9(2-hydroxy-3-nonyl)-purine hydrochloride, erythro-9-(2-hydroxy-3-nonyl)-3-deazaadenine, 1-deazaadenosine, Adenosine, 2-cyano-2′,3′-dideoxy-, Adenosine, 2′,3′-dideoxy-2-ethyl-, Adenosine, 2′,3′-dideoxy-2-(methylthio)-, Adenosine, 2′,3′-dideoxy-2-(trifluoromethyl)-, 2′,3′-Dideoxy-2-iodoadenosine, (+/−)-9H-Purine-9-ethanol, 6-amino-β-hexyl-α-methyl-, and analogs and combinations thereof In another specific embodiment of the present invention, the adenosine deaminase inhibitor can be (R)-3-(2-Deoxy-β-D-erythro-pentofuranosyl)-3,6,7,8-tetrahydroimidazo[4,5-d][1,3]diazepin-8-ol.

As used herein, “mammal” refers to an animal of the class Mammalia, e.g., human.

As used herein, a “lotion” refers to a liquid, usually an aqueous medicinal preparation containing one or more insoluble substances and applied externally for skin disorders; “cream” refers to an emulsified medicinal or cosmetic preparation; a semisolid emulsion of either the oil-in-water or the water-in-oil type, ordinarily intended for topical use; “gel” refers to a colloid in a more solid form than a solution; a jelly-like material formed by the coagulation of a colloidal liquid; many gels have a fibrous matrix and fluid filled interstices: gels are viscoelastic rather than simply viscous and can resist some mechanical stress without deformation; and “ointment” refers to a salve or unguent for application to the skin, specifically a semisolid medicinal preparation usually having a base of fatty or greasy material; an ointment has an oil base whereas a cream is water-soluble. (Webster's II New College Dictionary, Houghton Mifflin Company, New York (2001); Merriam Webster's Medical Desk Dictionary, Merriam-Webster. Incorporated, Springfield, Mass. (1996))

“Therapeutically effective amount” is intended to include an amount of a nucleoside analogue (e.g., dAd, cladribine and dCF) useful in the present invention or an amount of the combination of compounds claimed, e.g., to treat the skin disorder or treat the symptoms of the skin disorder in a host. The combination of compounds is preferably a synergistic combination. Synergy, as described for example by Chou and Talalay, Adv. Enzyme Regul., 22:27 (1984), occurs when the effect (in this case, treatment of skin disorder) of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

As used herein, “calamine” is a pink powder of zinc oxide and a skin protectant containing about 98% zinc oxide and about 0.5% ferric oxide; “aloe” is the dried latex of leaves of Curaco Aloe (Aloe barbadenis Miller, Aloe vera Linne) or Cape Aloe (Aloe ferox Miller and hybrids), of the family Liliacaea; “Vitamin E” is 3,4-dihydro-2,5,7,8-tetramethyl-2-(4,8,12-trimethyltridecyl)-2H-1-benzopyran-6-ol; “Vitamin E acetate” is 3,4-dihydro-2,5,7,8-tetramethyl-2-(4,8,12-trimethyltridecyl)-2H-1-benzopyran-6-ol acetate; and “lanolin” is the fat-like secretion of the sebaceous glands of sheep (i.e., complex mixture of esters and polyesters of 33 high molecular weight alcohols and 36 fatty acids) which is deposited onto the wool fibers.

As used herein, “deoxycoformycin” or “pentostatin” refers to (R)-3-(2-Deoxy-β-D-erythro-pentofuranosyl)-3,6,7,8-tetrahydroimidazo[4,5-d][1,3]diazepin-8-ol.

As used herein, “coformycin” refers to a ribonucleoside antibiotic synergist and/or an adenosine deaminase inhibitor isolated from organisms such as Nocardia interforma or Streptomyces kaniharaensis. It is proposed that as an antineoplastic agent and immunosuppressant, Corformycin's activities include, but are not limited to, an antineoplastic, antineoplastic synergist, immunosuppressant, antibiotic, and enzyme inhibitor.

As used herein, “diethyl pyrocarbonate” refers to diethyl pyrocarbonate and diethyl dicarbonate. Its empirical formula is C₆H₁₀O₅.

As used herein, “erythro-9-(2-hydroxy-3-nonyl) adenine” (EHNA) refers to an adenosine deaminase inhibitor and/or an inhibitor of cyclic GMP-stimulated phosphodiesterase. Cladribine, erythro-6-amino-9(2-hydroxy-3-nonyl)-purine hydrochloride, erythro-9-(2-hydroxy-3-nonyl) adenine and erythro-9-[3-(2-hydroxynonyl)]adenosine refer to additional adenosine deaminase inhibitors.

As used herein, “6-(R)-hydroxyl-1,6-dihydropurine ribonucleoside” (HDPR) refers to a transition-state analogue inhibitor of adenosine deaminase that differs only by a single hydroxyl group at the C6 position of purine ribonucleoside.

Erythro-9-(2-hydroxy-3-nonyl)-3-deazaadenine, 1-deazaadenosine Adenosine, 2-cyano-2′,3′-dideoxy-, Adenosine, 2′,3′-dideoxy-2-ethyl-, Adenosine, 2′,3′-dideoxy-2-(methylthio)-, Adenosine, 2′,3′-dideoxy-2-(trifluoromethyl)-, 2′,3′-Dideoxy-2-iodoadenosine, (+/−)-9H-Purine-9-ethanol, 6-amino-β-hexyl-α-methyl-, and analogs thereof are additional examples of adenosine deaminase (adenosine aminohydrolase, ADA) inhibitors.

As used herein, “skin absorption enhancer” or “skin penetration enhancer” refers to any substance that aids, assists, and/or increases the ability of a substance (e.g., dCF, dAdo, cladribine, or a combination thereof) to be absorbed into the skin surface of a mammal (e.g., human). “Skin absorption enhancer” or “skin penetration enhancer” also refers to any substance that speeds up the absorption of substances (e.g., dAd) through the skin. For example, a “skin absorption enhancer” or “skin penetration enhancer” may increase the amount of substance that is absorbed into the skin by about 10%, about 15%, about 25%, about 50%, about 75, about 100% or more, when compared to topical use of the substance (e.g., dAd) without a “skin absorption enhancer” or “skin penetration enhancer”. Suitable skin absorption/penetration enhancers include, e.g., diethylene glycol monoethyl ether (transcutol), dimethyl sulfoxide (DMSO), C₁₀DMSO, propyleneglycol, ionic surfactants, non-ionic surfactants, anionic surfactants, isopropyl myristate (IPM), calcipotriene, detergents, emollients, chelators (e.g., calcium chelators such as EDTA, EGTA), and combinations thereof. Additional Examples of enhancers include Loramide DEA, Ethoxydiglycol, NMP, Triacetin, Propylene Glycol, Benzyl Alcohol, Sodium Laureth Sulfate, Dimethyl Isosorbide, Isopropyl Myristate, Olive Squalane, Medium Chain Triglyceride Oil (MCT Oil), Menthol, Isopropyl Palmitate, Isopropyl Isostearate, Propylene Glycol Monostearate, Lecithin, Diisopropyl Adipate, Diethyl Sebacate, Oleic Acid, Ethyl Oleate, Urea, Glyceryl Oleate, Caprylic/Capric Triglyceride, Propylene Glycol Dicaprylate/Dicaprate, Laureth 4, Oleth-2, Oleth-20, Propylene Carbonate, Nonoxynol-9, 2-n-nonyl-1,3-dioxolane, C7 to C14-hydrocarbyl substituted 1,3-dioxolane, 1,3-dioxane, or acetal, and Nonoxynol-15. Specifically, the skin absorption enhancer can include diethylene glycol monoethyl ether (transcutol). As used herein, “diethylene glycol monoethyl ether” or “transcutol” refers to 2-(2-ethoxyethoxy)ethanol [CAS NO. 001893].

As used herein, “keratolytic agent” refers to a substance that causes desquamation (loosening) and debridement or sloughing of the surface cells of the epidermis. Any suitable keratolytic agent can be employed, preferably the keratolytic agent effectively causes desquamation (loosening) and debridement or sloughing of the surface cells of the epidermis. Preferably, the keratolytic agent is pharmaceutically acceptable for topical use on humans. Suitable keratolytic agents include, e.g., alcloxa, resorcinol, or a combination thereof As used herein, “alcloxa” refers to Al-chlorhydroxy allontoinate; and “resorcinol” refers to m-dihydroxybenzene or 1,3-benzenediol. Any suitable and effective amount of keratolytic agent can be employed, provided the amount of keratolytic agent effectively causes desquamation (loosening) and debridement or sloughing of the surface cells of the epidermis. The keratolytic agent can include an amount of alkaline material (e.g., potassium hydroxide (KOH), sodium hydroxide (NaOH), etc.), effective to cause desquamation (loosening) and debridement or sloughing of the surface cells of the epidermis. Alternatively, the desquamation (loosening) and debridement or sloughing of the surface cells of the epidermis can be achieved with the use, e.g., of mechanical stripping, tape, etc. Alternatively, the desquamation (loosening) and debridement or sloughing of the surface cells of the epidermis can be achieved with the use, e.g., of radiant energy such as ultrasound, heat, etc., or with the use of photodynamic therapy.

As used herein, “adhesive skin patch” refers to an article of manufacture that includes a flexible backing having a front side and a back side and a formulation positioned on and/or in at least a portion of the front side of the backing. The formulation includes a therapeutically effective amount of the compositions described herein. The formulation will also typically include an adhesive on and/or in at least a portion of the front side of the backing, sufficient to adhere the adhesive skin patch directly to a skin surface. The adhesive skin patch can either be occlusive or non-occlusive. In one specific embodiment of the invention, the adhesive skin patch can be a transdermal adhesive skin patch.

As used herein, “essentially free of liquid” refers to less than about 10 wt. % liquid, less than about 1 wt. % liquid, less than about 0.5 wt. % liquid, or less than about 0.1 wt. % liquid.

As used herein, “essentially free of water” refers to less than about 10 wt. % water, less than about 1 wt. % water, less than about 0.5 wt. % water, or less than about 0.1 wt. % water.

As used herein, “liquid” refers to a substance, which at standard temperature and pressure, undergoes continuous deformation under a shearing stress; the substance exhibits a characteristic readiness to flow, little or no tendency to disperse, and relatively high incompressibility. Specifically, liquid includes water.

As used herein, “corticosteroid” includes any of the synthetic or naturally occurring substances with the general chemical structure of steroids. Corticosteroids affect carbohydrate metabolism, electrolyte levels and protein catabolism as well as immune responses, gluconeogenesis (glyconeogenesis) and gonad function. Glucocorticoids, such as cortisol, control carbohydrate, fat and protein metabolism and are anti-inflammatory by preventing phospholipid release, decreasing eosinophils action and a number of other mechanisms. Cortisone and hydrocortisone are used to treat Addison's disease, a disorder caused by underproduction of the adrenal cortex hormones. Mineralocorticoids (any of the group of C21 corticosteroids, including aldosterone) control electrolyte and water levels, mainly by promoting sodium retention in the kidney. These and synthetic steroids are used extensively to treat arthritis and other rheumatoid diseases, including rheumatic heart disease. They are also used in some cases of autoimmune diseases such as systemic lupus erythematosus, in severe allergic conditions such as asthma, in allergic and inflammatory eye disorders, and in some respiratory diseases. The anti-inflammatory, itch-suppressing, and vasoconstrictive properties of steroids make them useful when applied to the skin to relieve diseases such as eczema and psoriasis and insect bites. The most common natural hormones are corticosterone (C₂₁H₃₀O₄), cortisone (C₂₁H₂₈O₅, 17-hydroxy-11-dehydrocorticosterone) and aldosterone. Other examples of corticosteroids include prednisone, prednisolone, triamcinolone, and betamethasone.

As used herein, “calcipotriene” refers to a synthetic topical form of vitamin D. It is involved in the growth and development of skin cells. Topical calcipotriene is used to treat plaque psoriasis (psoriasis with scaly patches). Chemically, calcipotriene is (5Z,7E, 22E,24S)-24-cyclopropyl-9, 10-secochola-5,7,10(19), 22-tetraene-1alpha, 3 beta, 24-triol-, with the empirical formula C₂₇H₄₀O₃.

As used herein, “retinoid” refers to vitamin A or vitamin A-like compounds, including, but not limited to, retinoic acid (RA), a natural acidic derivative of vitamin A. Retinoids play a critical role in normal development, growth and differentiation by modulating the expression of target genes.

As used herein, “anthralin” refers to an anthraquinone (the 9, 10 quinone derivative of anthracene; anthraquinones can be made synthetically and also occur naturally in aloe, cascara sagrada, senna, and rhubarb; the antineoplastic mitoxantrone is a synthetic derivative) derivative that reduces DNA synthesis and mitotic activity in hyperplastic epidermis, restoring the normal rate of epidermal cell proliferation and keratinization; used topically in the treatment of psoriasis and other skin conditions (also called dithranol).

As used herein, “coal tar” refers to a viscous black liquid containing numerous organic compounds that is obtained by the destructive distillation of coal. Coal tar can be distilled into many fractions to yield a number of useful organic products, including benzene, toluene, xylene, naphthalene, anthracene, and phenanthrene. These substances, called the coal-tar crudes, form the starting point for the synthesis of numerous products-notably dyes, drugs, explosives, flavorings, perfumes, preservatives, synthetic resins, and paints and stains. Coal tar is used medically to treat eczema, psoriasis, seborrheic dermatitis, and other skin disorders.

As used herein, “salicylic acid” refers to 2-hydroxybenzoic acid (C₆H₄(OH)CO₂H), which is a colorless, crystalline organic carboxylic acid. Salicylic acid is used to treat many skin disorders, such as acne, dandruff, psoriasis, seborrheic dermatitis of the skin and scalp, calluses, corns, common warts, and plantar warts.

As used herein, “photochemotherapy with ultraviolet A (PUVA)” refers to a type of ultraviolet radiation treatment (phototherapy) used for severe skin diseases. PUVA is a combination treatment which consists of Psoralen (P) administration and then exposure of the skin to long wave ultraviolet radiation (UVA). Psoralens include compounds which make the skin temporarily sensitive to UVA.

As used herein, “phototherapy with UVB” refers to a type of radiation treatment or therapy involving exposure to ultraviolet B light (wavelength 280-315 nm).

As used herein, “anti-E-selectin” includes antibodies, or fragments thereof, which interact with E-selectin, a vascular addressin.

As used herein, “antiCD11a” refers to a monoclonal or polyclonal antibody which inhibits T cells. The term “antiCD11a” also includes humanized monoclonal antibodies against a subunit of integrin LFA-1expressed on T cells. By inhibiting interaction of LFA-1and its ligands, T-cell activation and trafficking into psoriatic plaques are decreased.

As used herein, “anti-CD25 basiliximab” refers to a chimeric (murine/human) monoclonal antibody (IgG_{1κ}), produced by recombinant DNA technology, that functions as an immunosuppressive agent (e.g., it can be used to lower the body's natural immunity in patients who receive transplants), specifically binding to and blocking the interleukin-2 receptor α-chain (IL-2Rα, also known as CD25 antigen) on the surface of activated T-lymphocytes. It is a glycoprotein obtained from fermentation of an established mouse myleoma cell line genetically engineered to express plasmids containing the human heavy and light chain constant region genes and mouse heavy and light chain variable region genes encoding the RFT5 antibody that binds selectively to the IL-2Rα.

As used herein, “daclizumab” refers to a group of medicines known as immunosuppressive agents (e.g., it can be used to lower the body's natural immunity in patients who receive transplants). “Daclizumab” includes an immusuppresive, humanized IgG1 monoclonal antibody produced by recombinant DNA technology that binds specifically to the alpha subunit (p55 alpha, CD25 or Tac subunit) of the human high-affinity interleukin-2 (IL-2) receptor that is expressed on the surface of activated lymphocytes.

As used herein, “Alefacept” refers to an immunosuppressive dimeric fusion protein that consists of the extracellular CD2-binding portion of the human leukocyte function antigen-3 (LFA-3) linked to the Fc (hinge, CH2 and CH3 domains) portion of human IgG1. One use for alefacept is the treatment of psoriasis.

As used herein, “Efalizumab” refers to an immunosuppressive recombinant humanized IgG1 kappa isotype monoclonal antibody that binds to human CD11a.

As used herein, “CTLA4-IG” refers to a soluble form of CTLA (a key regulator of the activity of the immune system in that it “turns off” the immune response after it has successfully cleared a bacterial or viral infection). Its uses include treating autoimmune diseases and organ transplant rejection.

As used herein, “Anti-CD2” refers to antibodies which recognize an epitope of CD2 antigen implicated in T cell activation.

As used herein, “anti-CD4” refers to antibodies which recognize an epitope of CD4 antigen.

As used herein, “etanercept” refers to a dimeric fusion protein consisting of the extracellular ligand-binding portion of the human 75 kilodalton (p75) tumor necrosis factor receptor (TNFR) linked to the Fc portion of human IgG1. The Fc component of etanercept contains the CH2 domain, the CH3 domain and hinge region, but not the CH1 domain of IgG1.

As used herein, “infliximab” refers to a chimeric IgG1κ monoclonal antibody with an approximate molecular weight of 149,100 daltons. It is composed of human constant and murine variable regions. Infliximab binds specifically to human tumor necrosis factor alpha (TNFα) with an association constant of 10¹⁰ M⁻¹.

As used herein, “adenosine nucleotide” refers to a molecule with a ribose sugar attached to an adenine base on one of the nitrogen atoms of the adenosine (Ado) heterocyclic adenosine structure and at least one phosphate attached to the exterior carbon of ribose (the carbon on the outside of the five member ring for ribose).

As used herein, “adenosine nucleoside” refers to a molecule with a ribose sugar attached via a C1 carbon of the ribose ring to an adenine base on the N9 nitrogen atom of the adenosine heterocyclic adenosine structure. (A base linked to a sugar is called a nucleoside; when a phosphate group is added, the base-sugar-phosphate is called a nucleotide).

As used herein, “adenosine base” refers to adenine, which is one of the purine bases used in forming nucleotides of DNA and RNA. Adenine forms adenosine, a nucleoside, when attached to ribose and deoxyadenosine when attached to deoxyribose, and it forms Adenosine triphosphate, a nucleotide, when one or more phosphate group is added to adenosine. Adenosine triphosphate is used in many known cellular metabolisms as one of the basic methods of transferring chemical energy between reactions.

As used herein, “deoxyadenosine” (dAdo) refers to an adenine molecule attached to a deoxyribose ring (dA; C₁₀H₁₃N₅O₃, 2′-deoxyadenosine). In other words, deoxyadenosine molecules are adenosine molecules which can be substituted in any position, but are lacking one hydroxyl group in the ribose part of the molecule. Included in the definition of deoxyadenosine are analogs thereof, including, but not limited to, cladribine, clofarabine and fludarabine (F-Ara-A).

Agents useful in the present invention include nucleoside arabinosides, including adenosine arabinoside, such a Ara-A. As used herein, “Ara-A” (Vidarabine) refers to an analog of 2′-deoxyadenosine (dAdo). See, Structure of AraA (Cheson et al.,: Nucleoside Analogs in Cancer Therapy. Marcel Dekker, Inc. 1997. ISBN 0-8247-9850-3). It differs from dAdo in that the sugar moiety is a 2′ epimer of the ribose or a cyclic arabinose. AraA is a purine nucleoside analog that markedly potentiates the apoptotic effects of dCF on lymphoid and monocytic cells. AraA is a relatively good substrate of ADA, and it is deaminated. However, in the presence of ADA inhibition, AraA is metabolized to the triphosphate Ara-ATP through a series of phosphorylation reactions. After phosphorylation, Ara-ATP is a potent inhibitor of DNA synthesis. AraA can enter into the dNTP pools that serve as substrates for DNA polymerases that replicate the DNA. The inhibition of DNA synthesis is thought to be mediated through a competitive inhibition with the normal dATP for the dATP binding site on polymerases α and β. By incorporation into the DNA synthesis, Ara-ATP additionally interferes with the continued elongation of DNA during replication and repair of DNA damage by chain termination. Ara-ATP can also inhibit RNR by binding to the control function of the enzyme causing depletion in dATP and dCTP pools and upregulating dCK. This causes a self-potentiating effect because the lower the concentrations of dCTP and dATP become the more likely Ara-A will be phosphorylated and inhibit DNA synthesis. AraA is approximately 100-fold more potent as compared to dAdo when used in combination with dCF; however, the mechanism by which AraA synergizes with (e.g., potentiates, or is potentiated by) dCF's actions is different than dAdo. AraA's mechanism of action involves its ability to directly interfere with DNA synthesis. Ara-ATP is a competitive inhibitor of dATP as a substrate for DNA polymerase. In addition to inhibiting DNA polymerase, Ara-ATP is incorporated into DNA. Thus, in cells exposed to dCF and AraA, apoptosis is triggered very efficiently as a result of the combined inhibition of ADA, DNA polymerase and the incorporation of Ara-ATP into DNA. (Niitsu et al., Blood, 96:1512).

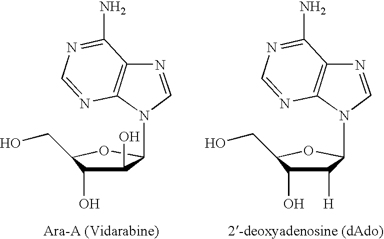

As used herein, “Ara-G” refers to an analog of deoxyguanosine (dGuo) that has resistance to catabolism by purine nucleoside phosphorylase (PNP). Ara-G has the advantage over Ara-A in that it is not susceptible to deamination by ADA. It differs from dGuo in that the sugar moiety is a 2′ epimer of the ribose or a cyclic arabinose. Below is the structure of Ara-G in comparison to dGuo.

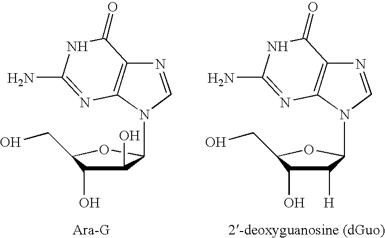

After phosphorylation, Ara-GTP is a potent inhibitor of DNA synthesis in the same aspect as Ara-ATP. Ara-GTP has specific activity in T-cells, as compared to B-cells. In particular, the selective toxicity to immature T-lymphoblasts is thought to occur through the incorporation into DNA synthesis as a chain terminator therefore hindering DNA elongation. In contrast to Ara-ATP, Ara-GTP has not been demonstrated to inhibit RNR. Because of its purine structural difference compared to Ara-A, the binding affinity to the RNR control-binding site is minimized.

As used herein, “Fludarabine” (F-ara-A) is a deoxyadenosine analog. Fludarabine was synthesized to be resistant to deamination by virtue of the presence of a halogen on the purine base as shown by the structure below. 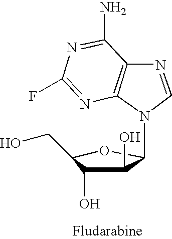
The active form of this purine analog is the triphosphate, F-Ara-ATP. As with Ara-A, F-Ara-ATP reduces DNA synthesis by incorporation into DNA by a DNA polymerase. Because F-Ara-ATP competes with the natural base, dATP, the cellular concentrations of dATP to F-Ara-ATP will be a strong determinant of the potential for inhibition of DNA synthesis. When F-Ara-ATP was studied upon incorporation into DNA synthesis, 95% was found at the 3′ terminus indicating a strong chain-terminating action. F-Ara-ATP also showed strong resistance to 3′→5′ excision, which is intrinsic to several DNA polymerases and acts as a first line of defense in correcting DNA polymerase errors. Additionally, F-Ara-ATP inhibits DNA ligase I that functions to join the 3′ end of one single DNA strand with the 5′ end of an immediately adjacent DNA strand annealed to the same DNA template. This is an essential natural function during DNA replication and the final step in repair of DNA damage. Not only does F-Ara-ATP inhibit DNA synthesis by incorporation into DNA polymerase and inhibiting repair, but also it effectively inhibits ribonucleotide reductase (RNR). RNR is the primary source for the production of deoxynucleoside triphosphates. This enzyme utilizes each ribonucleoside diphosphate as a substrate for the production of the corresponding deoxyribonucleoside diphosphate. The triphosphate quickly forms by the nucleoside diphosphate kinase. The concentration that gives 50% inhibition (IC₅₀) of RNR is approximately 0.6 μM. F-Ara-ATP creates a self-potentiating gradient after inhibiting RNR because of the direct decreases in the deoxynucleotides pools. Over the course of exposure to F-Ara-A, the competition will decrease between dATP with F-Ara-ATP for incorporation into DNA so the likelihood that F-Ara-ATP will inhibit DNA synthesis increases. In addition, RNR inhibition decreases dCTP levels. dCTP regulates phosphorylation by dCK and with decreasing dCTP levels dCK is upregulated generating greater amounts of F-Ara-ATP. In addition to the effect on DNA synthesis, F-Ara-ATP can incorporate into RNA. After incorporation into mRNA, F-Ara-ATP acts as a chain terminator and interferes with protein translation. The malignancies in which F-Ara-ATP demonstrated clinical activity are characterized by slow proliferating cells so it can have an effect on quiescent cells, such as chronic lymphoid leukemia.

As used herein, “Cladribine” (CdA, 2-CdA, Leustatin®, 2-chlorodeoxyadenosine, 2-chloro-2′-deoxy-β-D-adenosine, 2-chloro-2′-deoxyadenosine, 2-halo-2′-deoxyadenosine, NSC-105014-F, 2-chloro-6-amino-9-(2-deoxy-β-D-erythropento-furanosyl) purine) refers to a synthetic antineoplastic agent that is part of the second-generation nucleoside analogs. Cladribine is structurally related to fludarabine (it differs from Fludarabine in that it is of non-arabinose nature) and pentostatin, but has a different mechanism of action. It was synthesized to be resistant to deamination by ADA by the addition of a halogen group, specifically chlorine, at the 2 position of the purine ring, when compared to dAdo. This is illustrated by the below structure. 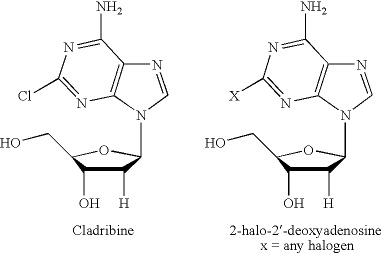
Cladribine is phosphorylated by dCK and dGK to the metabolically active nucleoside triphosphate, CdATP. This is accomplished significantly more efficient than for Fludarabine with a Kₘ for dCK of 5 μM and 80 μM for dGK. After phosphorylation, CdATP is incorporated into DNA synthesis more readily than F-Ara-ATP, but it is incorporated as an internal part of the DNA sequence instead of a chain terminator. In addition, CdATP has weak resistance to 3′ to 5′ excision so once CdATP is incorporated into the DNA the effect can be negated more efficiently. CdATP inhibits RNR but at concentrations approximately 10-times lower than that of F-Ara-ATP, IC50 of 0.06 μM. This leads to the same self-potentiating effects as seen with F-Ara-ATP. Once RNR is inhibited, dATP and dCTP levels will be depleted. This allows CdATP to be incorporated in to DNA synthesis more efficiently because there is less competition with dATP. The depletion of dCTP will cause an upregulation of dCK making CdA more susceptible to phosphorylation. Because of the internal incorporation of CdATP into DNA synthesis, the effect on quiescent cells may be greater than on dividing cells. In resting cells, elevated levels of CdATP will cause an accumulation of single strand DNA breaks that is presumed to activate the enzyme poly (ADP-ribose) polymerase (PARP). Upregulation of PARP leads to cellular loss of nicotinamide adenine dinucleotide (NAD), which is a cofactor in energy (ATP) production, resulting in ATP depletion and subsequent loss of cell function.

In recent studies Cladribine (CdA) is deaminated by adenosine deaminase to the novel metabolite, 2-chlorodeoxyinosine. (Bierau et al. Journal of Chromatography, B: Analytical Technologies in the Biomedical and Life Sciences, 805(2) (2004)) Since CdA can be deaminated by ADA in the presence of dAdo, it is required to bind to the enzyme, and it necessarily follows that it competitively inhibits the deamination of dAdo. Therefore, by definition, CdA is an ADA inhibitor.

As used herein, “Nelarabine” (506U78) refers to a second-generation purine nucleotide that gets it effectiveness from the subsequent deamination by ADA. Nelarabine is a prodrug of Ara-G that was synthesized to increase its solubility in water and make it a clinically useful water-soluble prodrug of Ara-G. Nelarabine is not active itself, but demethoxylation of nelarabine by ADA converts it to the biologically active Ara-G. The Kₘ of Nelarabine to ADA is less than 1% that of adenosine, however, the high specific activity of this enzyme in lymphocytes results in rapid conversion. This reaction is shown below. 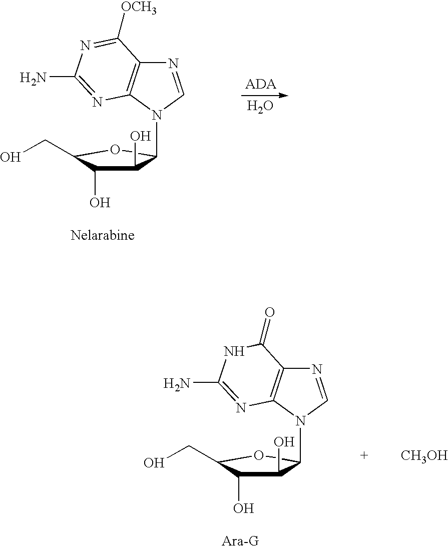
The use of Nelarabine in T-cell specific diseases makes it even more specific because the need for ADA deamination before phosphorylation to Ara-ATP. After conversion to Ara-G, phosphorylation to the active Ara-GTP inhibits DNA synthesis by chain termination.

As used herein, “Clofarabine” (Cl-F-ara-A; CAFdA) refers to a deoxyadenosine analog. Clofarabine, a third generation purine nucleoside analogue, incorporates the active properties of fludarabine and cladribine. It is resistant to deamination due to the addition of a —Cl group at the 2 position of the purine ring and it contains a 2′-fluro-arabinosyladenine, which is thought to obtain additional activity against DNA synthesis. The compound is illustrated below. 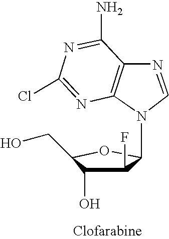
Clofarabine is phosphorylated by the enzyme dCK at the same efficiency as Cladribine, Kₘ of 5 μM and the mechanistic action is similar (its mechanism of action includes inhibition of DNA polymerase α and ribonucleotide reductase, leading to the depletion of intracellular deoxynucleotide triphosphate pools, disruption of mitochondrial function and apoptosis). The advantage to Clofarabine is that the arabinose fluorine group makes it more stable at a lower pH and less prone to degradation in semi-solid formulations. This makes it desirable for use in a finished product due to the increased stability.

As used herein, “Ara-C” (Cytarabine) refers to an analog of 2′-deoxycytarabine (dCyt) which was one of the first pyrimidine nucleoside analogs studied in T-cell type diseases. Ara-C undergoes deamination by cytidine deaminase (CDA), but not by ADA. Unlike Ara-A, Ara-C is less prone to deamination due to the lower concentration of CDA compared to ADA in lymphocytes. Ara-C derives its activity from the arabinosyl carbohydrate moiety and is metabolized to Ara-CTP by phosphorylation with dCK. The Kₘ of Ara-C for dCK is 10 μM. 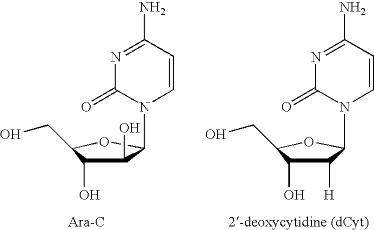
Ara-CTP competes with dCTP for incorporation into the DNA synthesis. Ara-CTP has strong inhibitory effects on DNA polymerases because Ara-CTP is incorporated into the terminus of the elongating DNA strand. Ara-CTP can also have fairly strong resistance to 3′→5′ excision making it inhibitory to DNA repair. Ara-C does not have any self-potentiating effects mostly because it does not inhibit RNR, although Ara-C in combination with RNR inhibitors have shown effects that are 100-times stronger than Ara-C alone. Another limitation with Ara-CTP is the rapid elimination of the triphosphate form.

As used herein, “Gemcitabine” (dFdC) refers to another pyrimidine analog of dCyt which has two fluorines placed in the geminal configuration on the 2′ carbon of the sugar base. Gemcitabine also undergoes rapid deamination by CDA systemically with a half-life in the range of 10-20 minutes because CDA has high specific activity in large organs such as liver, spleen, and kidneys. 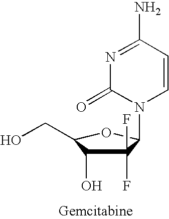
Gemcitabine is phosphorylated at a relatively high rate. Gemcitabine is active in both the di- and tri-phosphate forms. As a triphosphate, gemcitabine has the most affinity for incorporation into the internal position of replicating DNA. While this is not done efficiently, once the triphosphate is incorporated it is extremely resistant to 3′→5′ excision. In contrast, the diphosphate of gemcitabine is the metabolite that inhibits RNR. The respective IC₅₀ is 0.2 μM. Although gemcitabine is susceptible to deamination by CDA, once it is phosphorylated to its respective nucleoside phosphate the self-potentiating properties cause an exponential effect on specific cells. After gemcitabine diphosphate starts to inhibit RNR, the levels of dCTP become decreased. This leads to an upregulation of dCK that will increase gemcitabine potential to phosphorylate. These phosphates are eliminated by dCMP deaminase and require dCTP for activation, so with decreasing levels of dCTP from inhibition of RNR there becomes a prolonged terminal half-life. A self-potentiating effect of the triphosphate form is its likeliness to inhibit CTP synthetase, which is a metabolic enzyme in the production of CTP. The decreased concentration of CTP will ultimately lower the dCTP levels, further enhancing the activity of gemcitabine. A combination product that was resistant to deamination and inhibited RNR efficiently (cladribine) could first be used to decrease the levels of dCTP enough so that when gemcitabine was added the effect on cells would be immediate.

Pyrimidine compounds of use in the invention include, but are not limited to, fluoromethylenedeoxycytidine and troxacitabine. As used herein, “fluoromethylenedeoxycytidine” refers to ((E)-2′-deoxy-2′-(fluoromethylene)-cytidine; FMdC) a deoxycytidine analog displaying a very high toxicity toward a variety of solid tumor cell lines and xenografts. It is activated intracellularly by deoxycytidine kinase (dCK). As used herein, “troxacitabine” (TROX) refers to a dioxolane L-nucleoside analog with broad cytotoxic activity in in vitro and in vivo models. Other than its unique stereochemistry, troxacitabine has distinct pharmacology: it undergoes intracellular phosphorylation (predominant intracellular form is the diphosphate), but is resistant to deamination and is a complete DNA chain terminator.

Additional, agents useful in the present invention include halogenated nucleosides, nucleotides, purine bases, pyrimidine bases, and analogues thereof, including nucleosides (e.g., deoxyadenosine), nucleotides, and bases (e.g., adenine) and analogues thereof halogenated with fluorine (F-Ara-A), chlorine (e.g., cladribine), bromine, iodine and/or astatine, including, but not limited to, 2-halo-deoxyadenosines, e.g., 2-chloro-deoxyadenosine, 2-bromo-deoxyadenosine, and 2-fluoro-deoxyadenosine. Additionally, halogenated, as well as non-halogenated, nucleoside arabinosides are included within the scope of the invention, including, but not limited to, 2-halo-Ara-A, 2-halo-Ara-C and 2-halo-Ara-G, and analogs thereof.

As used herein, “lymphocyte” refers to a class of leukocyte produced in a variety of lymphoid organs throughout the body that is responsible for cellular and humoral immune responses. Lymphocytes are a type of white blood cell and can be divided into two main classes, T lymphocytes and B lymphocytes. T-cells are responsible for cell mediated immunity, whereas B-cells are responsible for humoral immunity (relating to antibodies). In the presence of an antigen, B-cells become much more metabolically active and transform into plasma cells. Plasma cells are large lymphocytes with a large nuclear to cytoplasmic ratio and are the form of B-cell lymphocytes that produce antibodies.

Lymphocytes play an important and integral part of the body's defenses. For example, lymphocytes are often seen at sites of infection and chronic inflammation. They produce many secretory products (lymphokines) that modulate the functional activities of a wide variety of cell types. A lymphocyte count is part of a peripheral complete blood cell count and is expressed as percentage of lymphocytes to total white blood cells counted. A general increase in the number of lymphocytes is known as lymphocytosis whereas a decrease is lymphocytopenia. (Kuby, Janis ( 1992), Immunology, New York: W. H. Freeman and Co.; Goldsby et al. (1992), Immunology, Fifth Edition, New York: W. H. Freeman and Co.; Abbas and Lichtman (2003), Cellular and Molecular Immunology, Fifth Edition, Elsevier Science, Saunders)

As used herein, “monocyte” refers to a type of white blood cell that has a single nucleus and can ingest (take in) foreign material. (In other words, a monocyte is a mononuclear phagocyte that circulates in the blood.) Monocytes can emigrate from blood into the tissues of the body and there differentiate (evolve into) into cells called macrophages which play an important role in killing of some bacteria, protozoa, and tumor cells, release substances that stimulate other cells of the immune system, and are involved in antigen presentation. (Kuby, Janis ( 1992), Immunology, New York: W. H. Freeman and Co.; Goldsby et al. (1992), Immunology, Fifth Edition, New York: W. H. Freeman and Co.; Abbas and Lichtman (2003), Cellular and Molecular Immunology, Fifth Edition, Elsevier Science, Saunders)

As used herein, “macrophages” refers to any of the many forms of mononuclear phagocytes found in tissues. Mononuclear phagocytes arise from hematopoietic stem cells in the bone marrow. After passing through the monoblast and promonocyte states of the monocyte stage, they enter the blood, circulating for about 40 hours. They then enter tissues and increase in size, phagocytic activity, and lysosomal enzyme content and become macrophages. The morphology of macrophages varies among different tissues and between normal and pathologic states, and not all macrophages can be identified by morphology alone. However, most macrophages are large cells with a round or indented nucleus, a well-developed Golgi apparatus, abundant endocytotic vacuoles, lysosomes, and phagolysosomes, and a plasma membrane covered with ruffles or microvilli. Among the functions of macrophages are nonspecific phagocytosis and pinocytosis, specific phagocytosis of opsonized microorganisms mediated by Fc receptors and complement receptors, killing of ingested microorganisms, digestion and presentation of antigens to T and B lymphocytes, and secretion of a large number of diverse products, including many enzymes (lysozyme, collagenases, elastase, acid hydrolases), several complement components and coagulation factors, some prostaglandins and leukotrienes, and several regulatory molecules (interferon, interleukin-1). Among the cells now recognized as macrophages are histiocytes, Kupffer cells, osteoclasts, microglial cells, synovial type A cells, interdigitating cells, and Langerhans cells (in normal tissues) and epithelioid cells and Langerhans-type and foreign-body-type multinucleated giant cells (in inflamed tissues). (Kuby, Janis ( 1992), Immunology, New York: W. H. Freeman and Co.; Goldsby et al. (1992), Immunology, Fifth Edition, New York: W. H. Freeman and Co.; Abbas and Lichtman (2003), Cellular and Molecular Immunology, Fifth Edition, Elsevier Science, Saunders)

As used herein, “dendritic cells” refers to immune cells which form part of the immune system. Multiple types of dendritic cells form from monocytes, white blood cells which circulate in the body and, depending on the right signal, can turn into dendritic cells or macrophages. Dendritic cells are present in those tissues which are in contact with the environment: in the skin (where they are often called Langerhans cells) and the lining of nose, lungs, stomach and intestines. They have “long spiky arms” called dendrites. Dendritic cells constantly sample the surroundings for viruses and bacteria Once they have captured an invader, they cut its proteins into small pieces and present those fragments at their cell surface using MHC molecules. They then travel through the blood stream to the spleen or through the lymphatic system to a lymph node. Here they act as antigen presenting cells: they activate helper T-cells (every helper T-cell is specific to one particular antigen; usually, only dendritic cells are able to activate a helper T-cell which has never encountered its antigen before) and killer T-cells as well as B-cells by presenting them with the pieces of the invader. Depending on the type of invader, this results in an immune response involving antibodies or killer cells. (Kuby, Janis ( 1992), Immunology, New York: W. H. Freeman and Co.; Goldsby et al. (1992), Immunology, Fifth Edition, New York: W. H. Freeman and Co.; Abbas and Lichtman (2003), Cellular and Molecular Immunology, Fifth Edition, Elsevier Science, Saunders).

As used herein, “Langerhans cells” (LCs) refer to the main type of dendritic cell (DC) found in the epidermis, although it should be noted that they do not appear to be preferentially localized to psoriatic lesions (Wollenberg, A et al., 2002. J Invest Dermatol., 119:1096-1102). They have the phenotype of immature DCs, including low expression of CD80 (B7-1), CD83, CD86 (B7-2) and ICAM-1, but are distinguished from other DC populations by the expression of CD1a and langerin (CD207) and the presence of Birbeck granules (Geissmann, F. et al., 1998. J. Exp. Med., 187:961-966; Charbonnier, A. S. et al., 1999. J Exp. Med., 190:1755-1767; Dieu-Nosjean, M. C. et al., 2000. J. Exp. Med., 192:705-717). LCs are believed to play a key role in capturing antigens in the skin and migrating to lymph nodes to present these antigens to T cells. Human LCs have been prepared from CD14+ monocytes by culture in the presence of GM-CSF, IL4 and TGF-β (Geissmann, F. et al., 1998. J. Exp. Med., 187:961-966). TGF-β has been shown to be a key cytokine for LC development, as TGF-β-null mice lack epidermal LCs (Borkowski, T. A. et al., 1996. J Exp Med., 184:2417-2422). LCs have also been prepared from murine bone marrow by culture with TGF-β (Zhang, Y. et al., 1999. Blood, 93:1208-1220).

As used herein, “plasmacytoid DCs” (pDCs) refers to cells which were originally described as a rare subpopulation of peripheral blood mononuclear cells (PBMCs) that produced very high amounts of type I interferons (IFN α and β) upon viral infection (the natural type I IFN producing cell) (Cella, M. et al., 2000. Nat. Med. 5:919-923; Siegal, F. P. et al., 2000. Science, 284:1835-1837). Using antibodies to the pDC markers CD123 and BDCA-2, they were found in the epidermis of lesions from a variety of inflammatory skin conditions, including psoriasis, contact dermatitis and lupus erythematosus, though they were absent in normal skin and atopic dermatitis (Wollenberg, A et al., 2002. J Invest Dermatol., 119:1096-1102). In mice, pDCs have been prepared from bone marrow by growth in the presence of Flt3 ligand (Boonstra, A. et al., 2003. J Exp Med, 197:101-109) and their numbers can be increased greatly in bone marrow and spleen by treating mice in -vivo with Flt 3 ligand and GM-CSF (Bjorck, P. 2001. Blood, 98:3520-3526). pDCs have been shown to be capable of inducing both Th1 and Th2 effectors depending on the antigen dose and activation signal (Boonstra, A. et al., 2003. J Exp Med. 197:101-109).

As used herein, “inflammatory dendritic epidermal cells” (IDECs) refers to cells that differ from classical LCs by lacking Birback granules and in expressing FceRI and CD11b. Like pDCs, they are present at elevated levels in skin lesions from psoriasis and contact dermatitis, but unlike pDCs, they are also found in atopic dermatitis lesions (Wollenberg, A et al., 2002. J Invest Dermatol., 119:1096-1102; Bowcock, A. M. et al. 2001. Human Molecular Genetics, 17:1793-1805).

As used herein, “synergize” or “synergizes” or “synergistic” refers to the working together of two substances (e.g., dCF and dAdo) to produce an effect greater than the sum of their individual effects (Webster's II New Collage Dictionary, Houghton Mifflin Company, New York (2001); Merriam Webster's Medical Desk Dictionary, Merriam-Webster. Incorporated, Springfield, Mass. (1996))

As used herein, “potentiate” or “potentiates” refers to the ability of one substance to make another substance (e.g., of one drug to make a second drug) effective or active or more effective or more active (Webster's II New Collage Dictionary, Houghton Mifflin Company, New York (2001); Merriam Webster's Medical Desk Dictionary, Merriam-Webster. Incorporated, Springfield, Mass. (1996)).

The term “prodrug” as used herein refers to any compound that when administered to a biological system generates the drug substance, i.e. active ingredient, as a result of spontaneous chemical reaction(s), enzyme catalyzed chemical reaction(s), photolysis, and/or metabolic chemical reaction(s). A prodrug is thus a covalently modified analog or latent form of a therapeutically-active compound. A prodrug may include an active metabolite (e.g., any substance involved in metabolism (either as a product of metabolism or as necessary for metabolism) or the drug itself.

As used herein, “antimicrobial preservative” refers to any number of compounds which inhibits mold, mildew, fungus, and/or bacteria growth in or on items, including drugs.

As used herein, “alleviate” refers to a physical or mental lightening, lessening, eliminating or diminishing of the severity or length of time of a condition or symptom underlying the condition.

As used herein, “chronic” refers to a condition, symptom or disease which persists over a long period of time and/or is marked by frequent recurrence (e.g., chronic colitis). Chronic disease refers to a disease which is of long continuance, or progresses slowly, in distinction from an acute disease, which quickly terminates.

As used herein, “skin disorder” refers to disorders of the skin including, but not limited to, disease of the skin, skin condition, skin disease, skin problems, which include, but are not limited to, acne, eczema, psoriasis, rosacea, skin cancer, skin burns, skin allergies, congenital skin disorders, acantholysis, acanthosis, acanthosis nigricans, dermatosis, disease, erythroderma, furunculosis, impetigo, jungle rot, keratoderma, keratodermia, keratonosis, keratosis, keratosis nigricans, leukoderma, lichen, livedo, lupus, melanism, melanosis, molluscum, necrobiosis lipoidica, necrobiosis lipoidica diabeticorum, pemphigus, prurigo, rhagades, Saint Anthony's fire, seborrhea, vitiligo, xanthoma, xanthosis, Psoriatic arthritis, Reiter's syndrome, Guttate psoriasis, Dyshidriotic eczema, Acute and chronic graft versus host disease, Systemic sclerosis, Morphea, Spongiotic dermatitis, Allergic dermatitis, Nummular eczema, Pityriasis rosacea, Pityriasis rubra pilaris, Pemphigus erythematosus, Pemphigus vulgaris, Lichenoid keratosis, Lichenoid nitidus, Lichen planus, Lichenoid dermatitis, Seborrheic dermatitis, Autosensitization dermatitis, Dermatitis herpetiformis, and Eosinophilic dermatitis. In one specific embodiment, the skin disorder can be mediated by an immunological response. In another specific embodiment, the skin disorder can be a lymphocyte-mediated skin disorder. In another specific embodiment, the skin disorder can be selected from the group of alopecia areata, psoriasis, atopic dermatitis, lupus erythematosis, bullous pemphigoid, psoriatic plaque, and combinations thereof. In another specific embodiment, the skin disorder can be psoriasis. In another specific embodiment, the skin disorder can be a chronic skin disorder. In another specific embodiment, the skin disorder can be an autoimmune skin disorder. In another specific embodiment, the skin disorder can be a malignant lymphoid disease that manifests in the skin.

As used herein, “emulsifying agent” refers to any substance that coats the particles of the dispersed phase and prevents coagulation of colloidal particles; an emulsifier.

As used herein, “solublizing agent” refers to any agent that can make a substance soluble or more soluble in another substance, especially in water.

As used herein, “humectant” refers to any substance that promotes retention of moisture.

As used herein, “ointment base” refers to any highly viscous or semisolid substance used, for example, in cosmetics, emollients, medicaments or salves.

As used herein, “solvent” refers to a substance, usually a liquid, capable of dissolving another substance, e.g., a solid substance or semi-solid substance.

As used herein, “viscosity-inducing agent” refers to any agent which can increase the viscosity of a solution/substance. Viscosity-inducing agents include, but are not limited to, water soluble natural gums, cellulose-derived polymers and the like.

As used herein, “wetting agent” refers to a substance that reduces the surface tension of a liquid, causing the liquid to spread across or penetrate more easily the surface of a solid.

As used herein, “mineral oil” refers to any of various light hydrocarbon oils, especially a distillate of petroleum.

As used herein, “propylene glycol” refers to colorless, viscous, hygroscopic liquid, CH₃CHOHCH₂OH, used in antifreeze solutions, in hydraulic fluids, and as a solvent.

As used herein, “wax” refers to any of various natural, oily or greasy heat-sensitive substances, consisting of hydrocarbons or esters of fatty acids that are insoluble in water but soluble in nonpolar organic solvents.

As used herein, “lyophilized” refers to drying and/or freezing of substances in a high vacuum to remove water, moisture or liquid therein (e.g., water content less than 5 wt. %, less than 1 wt. %, or less than 0.5 wt. %). Lyophilized also refers to freeze-dry.

As used herein, “itching” refers to an irritating skin sensation causing a desire to scratch.

As used herein, “inflammation” refers to a localized protective reaction of tissue to irritation, injury, or infection, characterized by pain, redness, swelling, and/or sometimes loss of function.

As used herein, “pain” refers to an unpleasant sensation occurring in varying degrees of severity as a consequence of injury, disease, or emotional disorder.

As used herein, an “analgesic” is a topically (i.e., externally) applied agent that relieves pain by altering perception of nociceptive stimuli without producing anesthesia or loss of consciousness; an “antipruritic” is a topically (i.e., externally) applied agent that prevents or relieves itching; and an “anesthetic” is a topically (i.e., externally) applied agent that can reversibly depress neuronal function, producing loss of ability to perceive pain and/or other sensations (see, Stedman's Medical Dictionary, 25th Ed., Ill., 1990, p. 65, p. 77, and p. 99).

The analgesic, anesthetic, or antipruritic can include one or more of camphor, menthol, benzocaine, butamben picrate, dibucaine, dibucaine hydrochloride, dimethisoquin hydrochloride, dyclonine hydrochloride, lidocaine, metacresol, lidocaine hydrochloride, pramoxine hydrochloride, tetracaine, tetracaine hydrochloride, benzyl alcohol, camphorated metacresol, juniper tar, phenol, phenolate sodium, resorcinol, diphenhydramine hydrochloride, tripelennamine hydrochloride, hydrocortisone, a corticosteroid, and hydrocortisone acetate. In one embodiment, the antipruritic can be camphor, menthol or a combination thereof. In another embodiment, the medicament can be lidocaine, hydrocortisone, or a combination thereof. In yet another embodiment, the medicament can be lidocaine, hydrocortisone, camphor, menthol or a combination thereof. The amount of the analgesic, anesthetic, or antipruritic will typically comply with Federal Register, Vol. 48, No. 27, § 348, and references cited therein. For example, as disclosed in Federal Register, Vol. 48, No. 27, § 348, camphor can be present up to about 3.0 wt. % of the therapeutic formulation and menthol can be present up to about 1.0 wt. % of the therapeutic formulation. In addition, benzocaine can be present in about 5.0 wt. % to about 20.0 wt. % of the therapeutic formulation. Butamben picrate can be present in about 0.5 wt. % to about 1.5 wt. % of the therapeutic formulation. Dibucaine can be present in about 0.25 wt. % to about 1.0 wt. % of the therapeutic formulation. Dibucaine hydrochloride can be present in about 0.25 wt. % to about 1.0 wt. % of the therapeutic formulation. Dimethisoquin hydrochloride can be present in about 0.3 wt. % to about 0.5 wt. % of the therapeutic formulation. Dyclonine hydrochloride can be present in about 0.5 wt. % to about 1.0 wt. % of the therapeutic formulation. Lidocaine can be present in about 0.5 wt. % to about 4.0 wt. % of the therapeutic formulation. Lidocaine hydrochloride can be present in about 0.5 wt. % to about 4.0 wt. % of the therapeutic formulation. Pramoxine hydrochloride can be present in about 0.5 wt. % to about 1.0 wt. % of the therapeutic formulation. Tetracaine can be present in about 1.0 wt. % to about 2.0 wt. % of the therapeutic formulation. Tetracaine hydrochloride can be present in about 1.0 wt. % to about 2.0 wt. % of the therapeutic formulation. Benzyl alcohol can be present in about 10.0 wt. % to about 33.0 wt. % of the therapeutic formulation. Camphor can be present in about 0.1 wt % to about 3.0 wt. % of the therapeutic formulation. Juniper tar can be present in about 1.0 wt. % to about 5.0 wt. % of the therapeutic formulation. Phenolate sodium can be present in about 0.5 wt. % to about 1.5 wt. % of the therapeutic formulation. Resorcinol can be present in about 0.5 wt. % to about 3.0 wt. % of the therapeutic formulation. Diphenhydramine hydrochloride can be present in about 1.0 wt. % to about 2.0 wt. % of the therapeutic formulation. Tripelennamine hydrochloride can be present in about 0.5 wt. % to about 2.0 wt. % of the therapeutic formulation. Hydrocortisone can be present in about 0.25 wt. % to about 1.0 wt. % of the therapeutic formulation. A corticosteroid can be present in about 0.25 to about 5.0 wt. % of the therapeutic formulation. Camphor can be present in about 3 wt. % to about 10.8 wt. % of the therapeutic formulation with phenol in accordance with Federal Register, Vol. 48, No. 27, § 348.20(a)(4). Camphor can be present in about 3 wt. % to about 10.8 wt. % of the therapeutic formulation with metacresol in about 1 wt. % to about 3.6 wt. % of the therapeutic formulation, as caphorated metacresol. In addition, hydrocortisone acetate can be present in about 0.25 wt. % to about 1.0 wt. % of the therapeutic formulation. See, e.g., Federal Register, Vol. 48, No. 27, § 348.

The therapeutic formulation can optionally include a topical moisturizer (i.e., skin conditioner). Any suitable topical moisturizer can be employed. Suitable topical moisturizers include, e.g., alpha hydroxy acid, a glycosaminoglycan, grape seed oil, cranberry seed oil, green tea, white tea, methylparaben, propylparaben, caffeine, xanthine, Vitamin B-3, nicotinamide, licorice, calamine, aluminum hydroxide gel, cocoa butter, aloe, lanolin, glycerin, Vitamin E, Vitamin E acetate, farnesol, glycyrrhetinic acid, propylene glycol, ethylene glycol, triethylene glycol, hard fat, kaolin, lanolin, mineral oil, petrolatum, topical starch, white petroleum, cod liver oil, shark liver oil, zinc oxide; or a combination thereof. Specifically, the topical moisturizer can be calamine, aloe, Vitamin E (i.e., tocopheryl), Vitamin E acetate (i.e., tocopheryl acetate), Vitamin C (i.e., L-(+)-ascorbic acid), lanolin, or a combination thereof. When employed, any suitable amount of topical moisturizer can be employed. The suitable amount of topical moisturizer will typically depend in part upon the specific moisturizer or moisturizers present in the therapeutic formulation. For example, the topical moisturizer (e.g., calamine, aloe, Vitamin E (i.e., tocopheryl), Vitamin E acetate (i.e., tocopheryl acetate), Vitamin C (i.e., L-(+)-ascorbic acid), lanolin, or a combination thereof) can be present up to about 40.0 wt. % of the therapeutic formulation, up to about 5.0 wt. % of the therapeutic formulation, or up to about 1.0 wt. % of the therapeutic formulation.

As used herein, “aluminum hydroxide gel” refers to a suspension containing aluminum oxide (Al₂O₃), mainly in the form of a hydroxide. It is typically obtained by drying the product of interaction in aqueous solution of an aluminum salt with ammonium or sodium carbonate.

As used herein, “cocoa butter” refers to a fatty substance in cocoa beans; a thick oily solid obtained from cocoa beans and used in making chocolate, cosmetics, and suntan oil. Also known as threobroma oil, it lubricates and softens the skin.

As used herein, “topical starch” refers to cornstarch.

As used herein, “kaolin” refers to aluminum silicate; powdered and freed from gritty particles by elutriation. Kaolin refers to the name of the locality in China where the substance is found in abundance.

As used herein, “white petroleum” refers to a purified mixture of hydrocarbons obtained from petroleum. A bleached version of yellow soft paraffin, it is used as an emollient and as a base for ointments. It is odorless when rubbed into the skin and not readily absorbed.

As used herein, “mineral oil” refers to the heavy liquid petrolatum; liquid paraffin or petroleum; a mixture of liquid hydrocarbons obtained from petroleum, and is typically used as a vehicle in pharmaceutical preparations.

As used herein, “petrolatum” refers to petroleum jelly, a yellow soft paraffin; a yellowish mixture of the softer members of the paraffin or methane series of hydrocarbons, obtained from petroleum as an intermediate product in the distillation; typically used as a soothing application to burns and abrasions of the skin, and as a base for ointments.

As used herein, “cod liver oil” refers to the partially destearinated fixed oil extracted from the fresh livers of Gadus morrhuae and other species of the family Gadidae, containing Vitamins A and D.

As used herein, “shark liver oil” refers to the oil extracted from the livers of sharks, mainly of the species Hypoprion brevirostris; a rich source of Vitamins A and D.

As used herein, “zinc oxide” refers to ZnO, which is typically used as a protective ointment.

As used herein, “calamine” is a pink powder of zinc oxide and a skin protectant containing about 98% zinc oxide and about 0.5% ferric oxide; “aloe” is the dried latex of leaves of Curaco Aloe (Aloe barbadenis Miller, Aloe vera Linne) or Cape Aloe (Aloe ferox Miller and hybrids), of the family Liliacaea. Aloe is commercially available as Aloe Vera Gel from Terry Laboratories (Melbourne, Fla.). Aloe Vera Gel is commercially available as Aloe Vera Gel 40× (20.0 wt. % solution in water), Aloe Vera Gel 1× (0.5 wt. % solution in water), Aloe Vera Gel 10× (5.0 wt. % solution in water), or solid Aloe Vera. The solid Aloe Vera can be dissolved in a carrier, such as water, to the desired concentration. In addition, the commercially available forms of Aloe Vera are optionally available as decolorized Aloe Vera.

As used herein, “Vitamin E” is 3,4-dihydro-2,5,7,8-tetramethyl-2-(4,8,12-trimethyltridecyl)-2H-1-benzopyran-6-ol; “Vitamin E acetate” is 3,4-dihydro-2,5,7,8-tetramethyl-2-(4,8,12-trimethyltridecyl)-2H-1-benzopyran-6-ol acetate; “lanolin” is the fat-like secretion of the sebaceous glands of sheep (i.e., complex mixture of esters and polyesters of 33 high molecular weight alcohols and 36 fatty acids) which is deposited onto the wool fibers; “farnesol” is 3,7,11-trimethyl-2,6,10-dodecatrien-1-ol. Farnesol is commercially available from American Radiolabeled Chemicals (ARC) (St. Louis, Mo.), and “glycyrrhetinic acid” is a pentacyclic triterpenoid derivative of the beta-amyrin type and is shown below: 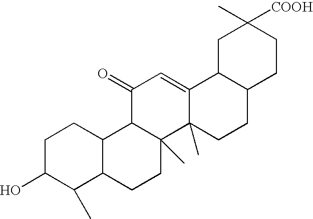
Nucleotides, Nucleoside, Bases and Analogs Thereof and Mechanisms of Their Action

Nucleoside transport systems provide the pathway for nucleoside analogs to cross the plasma membrane and enter cells. Available data suggests that nucleosides and nucleoside analogs are not limited in the transport mechanisms above approximately 1 μM. Data suggests that below 1 μM the rate-limiting step for the formation of the respective nucleotide will depend on how efficient the equilibrium system removes nucleosides from the cell in the process of the transportation of them into the cell. Once inside the cell, the nucleosides and/or analogs thereof will require phosphorylation to the respective nucleotide to be biologically active. Phosphorylation is accelerated through deoxyribonucleoside (dRNS) enzymes, the most universal being deoxycytidine kinase (dCK). After phosphorylation, the nucleosides and/or analogs thereof demonstrate elimination kinetics ranging from 2 to 30 hours. For the purine nucleosides, the elimination time is much slower than the pyrimidine congeners. This increased residence time creates prolonged intracellular presence and makes them valuable in lymphocyte-mediated diseases.

In comparison to other functional cells, levels of dCK are high in lymphocytes. Among the lymphocytes, dCK is expressed in T-cells considerably higher than in B-cells, which is marked by higher phosphorylating activity. In addition, 5′-nucleotidease (5NT) can dephosphorylate the nucleotide analogs to their respective nucleoside, but in lymphocytes the ratio of dCK to 5NT is significantly elevated. Another enzyme present in lymphocytes is adenosine deaminase (ADA). ADA has the ability to deaminate the purines adenosine and 2′-deoxyadenosine to their respective metabolites, inosine and 2′-deoxyinosine, and thus, some of the analogs susceptible to deamination by ADA.

Nucleosides analogs have two mechanisms of action, which are inhibition of ribonucleotide reductase (RNR) and/or DNA synthesis. The triphosphate nucleoside analogs can possess either or both of these mechanisms to differing degrees. In certain cases, the inhibition of one of these mechanisms will lead to a self-potentiation effect on the second mechanism. Below is a general illustration on the potential effects and metabolism process of nucleoside analogs.

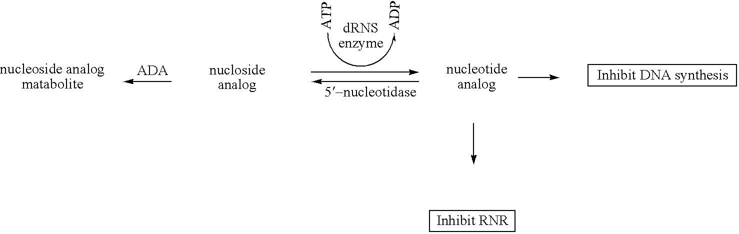

Suitable purine analogs include adenosine nucleotides, adenosine nucleosides and adenosine bases. Such purine analogs are disclosed, e.g., in EP 349242; U.S. Pat. No. 5,034,394; M. T. Crimmins, and B. W. King, J. Org. Chem. 61:4192 (1996); S. M. Daluge et al., Antimicrob. Ag. Chemother. 41:1082 (1997); R. H. Foster and D. Faulds, Drugs, 55:729-736 (1998); S. Staszewski et al., J. Am. Med. Assoc., 285:1155 (2001); Rosenberg et al., Coll. Czech. Chem. Commun. 53:2753 (1988); A. Holy et al., Coll. Czech. Chem. Commun., 60:1390 (1995); L. M. Schultze et al., Tetrahedron Letters, 39:1853 (1998); M. N. Arimilli et al., Antivir. Chem. Chemother., 8:557 (1997); WO 98 04569; U.S. Pat. No. 5,922,695; J. Balzarini et al., Biochem. Biophys. Res. Commun., 219:337 (1996); J.-P. Shaw et al., Pharm. Res., 14:1824 (1997); S. G. Deeks et al., Antimicrob. Ag. Chemother., 42:2380 (1998); W. Plunkett and S. S. Cohen, Cancer Res. 35:1547 (1975); EP 206497; R. R. Webb et al., Nucleosides Nucleotides, 7:147 (1988); H. Mitsuya and S. Broder, Proc. Nat. Acad. Sci. USA, 83:1911 (1986); D. D. Richman, Antimicrob. Ag. Chemother. 31:1879 (1987); J. E. Dahlberg et al., Proc. Nat. Acad. Sci. USA, 84:2469 (1987); G. Ahluwalia et al., Biochem. Pharmacol., 36:3797 (1987); G. Ray and E. Murrill, Anal. Letters, 20, 1815 (1987); J. S. Lambert et al., N. Engl. J. Med. 322:1333 (1990); T. P. Cooley et al., N. Engl. J. Med., 322:1340. M. N. Nassar et al., Anal. Profiles Drug Subs. Excip., 22:185-227 (1993); Kamel et al., Eur J Mass Spectrom (Chichester, Eng). 2004;10(2):239-57; Lee, Drugs Today (Barc). 1998 March; 34(3):241-9; Wilhelmus, Cochrane Database Syst Rev. 2003 (3):CD002898; Shiraki, Nippon Rinsho. 2003 February; 61 Suppl 2:792-7; U.S. Pat. No. 6,677,310; Niwa et al., Oncol Rep. 2003 September-October;10(5):1437-41; Hayashi et al., Histol Histopathol. 2003 October;18(4):1155-68; Spiers, Haematologia (Budap). 1996;27(2):55-84; U.S. Pat. No. 6,734,178; Henrot, Ann Dermatol Venereol. 2002 April; 129(4 Pt 2):533-49; Worthington, N Engl J Med. 2003 May 8;348(19):1920-5; author reply 1920-5; Chilukuri et al., Dermatol Clin. 2003 April; 21(2):311-20; Kleymann, Expert Opin Investig Drugs. 2003 February; 12(2):165-83; Klammer et al., Hautarzt. 2003 April,54(4):362-4; U.S. Pat. No. 6,509,320; Kiewe et al., Leuk Lymphoma. 2003 October; 44(10):1815-8; Junghanss et al., Br J Haematol. 2003 November; 123(4):662-70; Chosidow et al., Ann Dermatol Venereol. 2002 April; 129(4 Pt 2):635-45; Ishii et al., Intern Med. 2003 March; 42(3):250-4; Holdiness, Contact Dermatitis. 2001 May; 44(5):265-9; Wilhelmus, Trans Am Ophthalmol Soc. 2000;98:505-32; U.S. Pat. No. 6,392,085; Wilhelmus, Cochrane Database Syst Rev. 2001 (1):CD002898; U.S. Pat. No. 6,358,963; Josephson et al., Antivir Ther. 1996 August; 1(3):147-56; Spiers, Am J Ther. 1995 March; 2(3):196-216; U.S. Pat. No. 6,255,485; Wang et al., Zhonghua Shi Yan He Lin Chuang Bing Du Xue Za Zhi. 1998 March; 12(1):12-4; Thomas-Golbanov et al., Expert Opin Investig Drugs. 2001 July; 10(7):1279-89; U.S. Pat. No. 6,255,292; Hernández et al., Biopolymers. 2002;67(6):440-55; Morales et al., Am J Ophthalmol. 2001 July; 132(1):114-6; Field et al., Eur J Haematol. 2002 July; 69(1):54-7; Fraternale et al., Eur J Clin Invest. 2001 March; 31(3):248-52; Rossi et al., Br J Haematol. 2001 October; 115(1):58-62; Bordigoni et al., Clin Infect Dis. 2001 May 1; 32(9):1290-7; Kesson, Paediatr Drugs. 2001; 3(2):81-90; Kimura et al., J Pediatr Hematol Oncol. 2001 June-July; 23(5):294-9; Tran et al., Gastroenterol Clin Biol. 2000 December; 24(12):1234-5; RE37,045; Kamiyama et al., J Gen Virol. 2001 November; 82(Pt 11):2761-5; Fried et al., Antivir Ther. 1996 April;1(2):71-6; Aoki, Herpes. 2001 July; 8(2):41-5; Hambach et al., Bone Marrow Transplant. 2001 October; 28(7):705-7; Su et al., Zhonghua Shi Yan He Lin Chuang Bing Du Xue Za Zhi. 2002 March; 16(1):16-9; Abruzzo et al., Am J Surg Pathol. 2002 May; 26(5):630-6; U.S. Pat. No. 5,808,147; Magnani et al., J Leukoc Biol. 2003 November; 74(5):764-71; Jindra et al., Bone Marrow Transplant. 2003 May; 31(10):951-2; Stratta et al., Transplant Proc. 2004 May; 36(4):1082-3; Chi-Ming Chiu et al., J Ethnopharmacol. 2004 July; 23(1):63-8; Labetoull, J Fr Ophtalmol. 2004 May; 27(5):547-57; Hurwitz et al., Pediatr Transplant. 2004 June; 8(3):267-72; Andrei et al., Antiviral Res. 2004 March; 61(3):181-7; Clark et al., Transplantation. May 15, 2004; 77(9):1480; Whitley, Curr Opin Infect Dis. 2004 June; 17(3):243-6; Genova et al., Toxicol Appl Pharmacol. Jun. 1, 2004; 197(2):107-12; Cheshenko et al., Antimicrob Agents Chemother. 2004 June; 48(6):2025-36; Benarroch et al., J Biol Chem. May 19, 2004; De Clercq et al., Nucleosides Nucleotides Nucleic Acids. 2001 April-July; 20(4-7):271-85; De Clercq, Nucleosides Nucleotides Nucleic Acids. 2000 October-December; 19(10-12):1531-41; De Clercq et al., Rev Med Virol. 2001 November-December; 11(6):381-95; Berenguer et al., Proc Assoc Am Physicians. 1998 March-April; 110(2):98-112; Andrei et al., Antiviral Res. 2004 March; 61(3):181-7; De Clercq, J Clin Virol. 2004 June; 30(2):115-33; Maggs et al., Am J Vet Res. 2004 April; 65(4):399-403; Kim et al., Org Biomol Chem. Apr. 21, 2004; 2(8):1164-8; Satoh et al., Int J Radiat Oncol Biol Phys. Jun. 1, 2004; 59(2):562-71; Walling et al., J Infect Dis. May 1, 2004; 189(9):1656-63; Brodell et al., Postgrad Med. 2004 April; 115(4):63-5; Lee, Drugs Today (Barc). 1998 March; 34(3):241-9; Morin et al., Nucl Med Biol. 2004 July; 31(5):623-30; Bidanset et al., Antiviral Res. 2004 July; 63(1):61-4; Stratta et al., Transplant Proc. 2004 May; 36(4):1082-3; Hurwitz et al., Pediatr Transplant. 2004 June; 8(3):267-72; Maggon et al., Rev Med Virol. 2004 May-June; 14(3):149-68; Medeiros et al., J Clin Gastroenterol. 2004 January; 38(1):84-6; Ribeiro et al., Hepatology. 2003 August; 38(2):509-17; Romero-Gómez et al., Am J Gastroenterol. 2003 July; 98(7):1621-6; Seignères et al., Antimicrob Agents Chemother. 2003 June; 47(6):1842-52; Chong et al., Antivir Chem Chemother. 2002 March; 13(2): 115-28; Seignères et al., Hepatology. 2002 September; 36(3):710-22; Pramoolsinsup, J Gastroenterol Hepatol. 2002 February; 17 Suppl:S125-45; Sumpter et al., Antiviral Res. 2004 June; 62(3):111-20; De Clercq, J Clin Virol. 2004 June; 30(2):115-33, MacManus et al., AIDS. Mar. 5, 2004; 18(4):651-5; Blanckenberg et al., AIDS. Mar. 5, 2004; 18(4):631-40; J. A. Montgomery and K. Hewson, J. Med. Chem. 12, 498 (1969); J. A. Montgomery et al., J. Heterocycl. Chem. 16:157 (1979); U.S. Pat. No. 4,210,745 R. W. Brockman et al., Biochem. Pharmacol., 26:2193 (1977);. W. Brockman et al., Cancer Res., 40:3610 (1980); V. Gandhi et al., J. Chromatog., 413:293 (1987); U.S. Pat. No. 4,357,324; M. R. Hersh et al., Cancer Chemother. Pharmacol., 17:277 (1986); R. P. Warrell, Jr. and E. Berman, J. Clin. Oncol. 4:74 (1986); H. G. Chun et al., Cancer Treat. Rep. 70:1225 (1986); Sem. Oncol., 17:Suppl. 8, 1-78 (1990); Levene and Bass, Nucleic Acids, p. 163 (New York, 1931); Levene, Tipson, J. Biol. Chem., 94:809 (1932); Bredereck, Ber., 66, 198 (1933); Z. Physiol. Chem., 223:61 (1934); Gulland, Holiday, J. Chem. Soc., 765 (1936); Szent-Györgyi, J. Physiol., 68:213 (1930); Lythgoe et al., J. Chem. Soc,. 355 (1947);: Lythgoe et al., J. Chem. Soc., 965 (1948); Davoll et al., J. Chem. Soc., 967 (1948); H. Vorbrueggen and K. Krolikiewicz, Angew. Chem. Intl. Ed., 14, 421 (1975); T. F. Lai and R. E. Marsh, Acta Crystallogr., B28:1982 (1972). D. B. Davies and A. Rabczenko, J. Chem. Soc. Perkin Trans., 2:1703 (1975); Prog. Clin. Biol. Res., 230, 1-395 (1987); Levine et al., Biol Blood Marrow Transplant., 9(3):189-97 (2003); Feinstein et al., Biol Blood Marrow Transplant., 9(4):266-72 (2003); Maris et al., Blood, 102(6):2021-30 (2003); Georges et al., Int J Hematol., 76 Suppl 1:184-9 (2002); Murotani et al., Clin Lab Haematol., 24(5):303-6 (2002); Mielcarek et al., Blood, 102(2):756-62 (2003); Maris et al., Transfus Clin Biol., 8(3):231-4 (2001); Niederwieser et al., Blood, 101(4):1620-9 (2003); Kreiter et al., Bone Marrow Transplant., 28(2):157-61 (2001); Feinstein et al., Ann N Y Acad Sci., 938:328-37; discussion 337-9 (2001); Ruiz-Argüelles, Rev Invest Clin., 54(2):154-60 (2002); Tsimberidou et al., Cancer, 100(12):2583-91 (2004); Ferrajoli et al., Semin Oncol., 31(2 Suppl 4):60-5 (2004); Zent et al., Leuk Res., 28(5):495-507 (2004); de Leval et al., Am J Surg Pathol., 28(5):679-86 (2004); Hillmen, Semin Oncol., 31(1 Suppl 2):22-6 (2004); Tam et al., Cancer, 100(10):2181-9 (2004); Huynh et al., Clin Nucl Med., 29(4):275-6 (2004); Rossi et al., J Clin Oncol., 22(7):1260-7 (2004); Nabhan et al., Leuk Res., 5:429-42 (2004); Shvidel et al., Leuk Lymphoma, 44(11):1947-50 (2003); Erba HP., Curr Hematol Rep., 3(1):47-53 (2004); Lozanski et al., Blood, 103(9):3278-81 (2004); Tóthová et al., Neoplasma, 50(6):433-7 (2003); Hallek et al., MMW Fortschr Med., 145(27-28):46-9 (2003); Friedberg et al., Leuk Res., 28(2):139-47 (2004); Jones et al., Blood, 103(5):1855-61 (2004); Imagawa et al., Gan To Kagaku Ryoho, 31(2):267-70 (2004); Usman et al., J Pak Med Assoc., 53(10):496-7 (2003); Mamani-Matsuda et al., Br J Haematol., 124(2):141-50 (2004); Kiewe et al., Leuk Lymphoma, 44(10):1815-8 (2003); Braester et al., Leuk Lymphoma, 44(10):1835-6 (2003); Montillo et al., Leukemia, 18(1):57-62 (2004); Tiscornia et al., Leukemia, 18(1):48-56 (2004); Stiefelhagen, MMW Fortschr Med., 145(39):55-6 (2003); Frankel et al., Clin Cancer Res., 9(10 Pt 1):3555-61 (2003); Pahler et al., Clin Cancer Res. 2(12):4570-7 (2003); Schetelig et al., J Clin Oncol., 21(14):2747-53 (2003); Johnston et al., Oncogene, 22(51):8356-69 (2003); Schiavone et al., Eur J Haematol., 71(1):23-8 (2003); Giles et al., Cancer Chemother Pharmacol., 52(3):223-8 (2003); Pettitt, Br J Haematol., 121(5):692-702 (2003); Castagna et al., Blood, 102(6):2309; author reply 2309-10 (2003); Starczynski et al., Br J Haematol., 123(1):66-71 (2003); Dreger et al., Leukemia, 17(5):841-8 (2003); Wells et al., Ann Pharmacother., 37(5):671-4 (2003); Tournilhac et al., Blood, 103(1):363-5 (2004); Weiss et al., J Clin Oncol., 21(7):1278-84 (2003); Montserrat, Semin Oncol. 2003 February; 30(1 Suppl 2):34-9; Morabito et al., Haematologica. 2003 August; 88(8):864-73; Beyan et al., Haematologia (Budap). 2002; 32(3):287-90; Rubnitz et al., Leuk Res. 2004 April; 28(4):349-52; Holowiecki et al., Leukemia. 2004 May; 18(5):989-97; Juliusson et al., Br J Haematol. 2003 December; 123(5):810-8; Wrzesień-Kuś et al., Eur J Haematol. 2003 September; 71(3):155-62; Robak, Leuk Lymphoma. 2003 March; 44(3):391-409; Zwaan et al., Blood. 1 Nov. 2002; 100(9):3352-60; Robak et al., Neoplasma 2001; 48(3):203-7; Korycka et al., Leuk Lymphoma. 2003 September; 44(9):1549-55; Razzouk et al., Leukemia. 2001 September; 15(9):1326-30; Krance et al., J Clin Oncol. Jun. 1, 2001; 19(11):2804-11; Lindemalm et al,. Haematologica. 2003 March; 88(3):324-32; Korycka et al., Arch Immunol Ther Exp (Warsz). 2003; 51(1):61-7; Crews et al., J Clin Oncol. Oct. 15, 2002; 20(20):4217-24; Seshadri, Leuk Res. 2000 July; 24(7):637; Robak et al., Leuk Lymphoma. 2000 September; 39(1-2):121-9; Lech-Marańda et al., Eur J Haematol. 2000 November; 65(5):317-21; Schirmer et al., Leuk Res. 1999 December; 23(12):1121-6; Gordon et al., Leuk Res. 2000 October; 24(10):871-5; Al-Fiar et al., Leuk Res. 1999 August; 23(8):719-21; Korycka et al., Arch Immunol Ther Exp (Warsz). 2000; 48(4):293-9; Niitsu et al., Leuk Res. 2000 January; 24(1):1-9; Van Den Neste et al., Ann Hematol. 1998 January; 76(1):19-23; Saito et al., Clin Cancer Res. 2002 April; 8(4):1014-20; Martinelli et al., Haematologica. 1996 May-June; 81(3):286-7; Giralt et al., Blood. Jun. 15, 1997; 89(12):4531-6; Korycka et al., Eur J Haematol. 2000 May; 64(5):347-9; Gollard et al., Leuk Lymphoma. 1997 December; 28(1-2):183-5; Dann et al., J Clin Oncol. 1998 April; 16(4):1498-504; Aleskog et al., Eur J Haematol. 1999 May; 62(5):293-9; Korycka et al., Leuk Res. 1997 September; 21(9):857-65; Cheson et al., J Clin Oncol. 1999 August;17(8):2454-60; Kimby et al., Eur J Haematol. 2004 April; 72(4):292-5; Crews et al., J Pediatr Hematol Oncol. 2002 November; 24(8):677-80; Giralt et al., Blood. Feb. 1, 2001; 97(3):631-7; Mathew et al., Leukemia. 2000 July; 14(7):1314-5; Kornblau et al., Leukemia. 1996 October; 10(10):1563-9; Klippstein et al., J Cancer Res Clin Oncol. 2003 May,129(5):316-9; Konstantinov et al., J Cancer Res Clin Oncol. 2002 May; 128(5):271-8; Zulkowski et al., J Cancer Res Clin Oncol. 2002 February; 128(2):111-3; Ruffert, Zentralbl Chir. 1998; 123 Suppl 5:156-8; Balfour et al., Drugs. 2001;61(5):631-8; discussion 639-40; Brockmann et al., Arch Geschwulstforsch. 1989;59(5):341-6; Höffken et al., J Cancer Res Clin Oncol. 1998;124(11):627-32; Strumberg et al., Anticancer Drugs. 1996 June; 7(4):415-21; Mehta et al., Bone Marrow Trasplant. 2004 June; 3(11):1107-14; Ishitsuka et al., Eur J Haematol. 2004 April; 72(4):280-4; Matsuda et al., Rinsho Ketsueki. 2003 September; 44(9):946-51; Chow et al., J Pediatr. 2004 May; 144(5):669-71; Toubai et al., Acta Haematol. 2003;109(4):199-201; Lobe et al., Leukemia 2003 August; 17(8):1600-4; Carroll et al., Hematology (Am Soc Hematol Educ Program). 2003:102-31; Ma et al., Zhonghua Er Ke Za Zhi. 2003 December; 41(12):929-33; Stoneham et al., Br J Haematol. 2003 October:123(1):100-2; Lauten et al., Haematolozica. 2003 November; 88(11):1253-8; Fenaux et al., Best Pract Res Clin Haematol. 2003 September; 16(3):495-501; Dervieux et al., Clin Pharmacol Ther. 2003 June; 73(6):506-16; Sanz et al., Best Pract Res Clin Haematol. 2003 September; 16(3):433-51; Nygaard et al., Clin Pharmacol Ther. 2004 April; 75(4):274-81; Fehr et al., Ann Hematol. 2003 March; 82(3):193-6; Lennard et al., Br J Clin Pharmacol. 1993 October; 36(4):277-84; Efferth et al., Int J Oncol. 2003 August; 23(2):509-17; Schaeffeler et al., Leukemia. 2003 July; 17(7):1422-4; Colović et al., Chemotherapy. 2003 June; 49(3):138-45; Starý et al., Cas Lek Cesk. 2003;142(7):404-9; Schmiegelow et al., J Clin Oncol. Apr. 1, 2003; 21(7):1332-9; Oliveira et al., Braz J Med Biol Res. 2004 May; 37(5):649-58; Halonen et al., Acta Paediatr. 2003;92(1):37-42; Bostrom et al., Blood. May 15, 2003; 101(10):3809-17; Krawczuk-Rybak et al., Endokrynol Diabetol Chor Przemiany Materii Wieku Rozw. 2003;2(1):35-40; Sastry et al., Pediatr Hematol Oncol. 2003 March; 20(2):157-60; Nygaard et al., Leukemia. 2003 July; 17(7):1344-8; Cheok et al., Nat Genet. 2003 May; 34(1):85-90; Harms et al., Blood. Oct. 15, 2003; 102(8):2736-40; Marshall E., Science. Oct. 24, 2003; 302(5645):588-90; De Abreu et al., Ann Clin Biochem. 2003 January; 40(Pt 1):70-4; Krawczuk-Rybak et al., Endokrynol Diabetol Chor Przemiany Materii Wieku Rozw. 2003;9(1):41-7; Ordentlich et al., J Biol Chem. Jul. 4, 2003; 278(27):24791-9; Martin et al., Pharmacotherapy. 2003 February; 23(2):260-4; Bessard et al., Therapie. 2004 January-February; 59(1):71-5; Benessahraoui et al., Arch Pediatr. 2003 October; 10(10):891-4; Rogalski et al., J Dermatol. 2003 January; 30(1):59-63; Soo et al., J Natl Cancer Inst. May 19, 2004; 96(10):796-800; Yamane et al., Biochem Biophys Res Commun. May 21, 2004; 318(1):297-302; Rooseboom et al., Pharmacol Rev. 2004 March; 56(1):53-102; Lebwohl et al., J Am Acad Dermatol. 2004 March; 50(3):416-30; Gunnarsdottir et al., Drug Metab Dispos. 2004 March; 32(3):321-7; Lange et al., J Clin Oncol. Jan. 1, 2004; 22(1):150-6; Bakshi et al., Indian J Pediatr. 2003 September; 70(9):755-8; Krynetski et al., Oncogene. Oct. 20, 2003; 22(47):7403-13; Wiernik et al., Leuk Lymphoma. 2003 September; 44(9):1515-21; Stoneham et al., Br J Haematol. 2003 October; 123(1):100-2; Heath et al., Pediatr Hematol Oncol. 2003 October-November; 20(7):497-504; Gamis et al., J Clin Oncol. Sep, 15, 2003; 21(18):3415-22; Vernole et al., Cancer Chemother Pharmacol. 2003 September; 52(3):185-92; Dervieux et al., Clin Pharmacol Ther. 2003 June; 73(6):506-16; Yan et al., Clin Cancer Res. 2003 June; 9(6):2327-34; Jess et al., Aliment Pharmacol Ther. Feb. 1, 2004; 19(3):287-93; Taylor et al., Eur J Neurol. 2004 February; 11(2):141; Gainon et al., Schweiz Rundsch Med Prax. Oct, 8, 2003; 92(41):1734-9; Balal et al., Transplant Proc. 2003 June; 35(4):1408-9; Maltzman et al., J Clin Invest. 2003 April; 111(8):1122-4; Rammert et al., Ther Umsch. 2003 March; 60(3):145-50; Alvarez Delgado et al., Gastroenterol Hepatol. 2003 January; 26(1):52-3; Wang et al., Clin Transplant. 2002 December; 16(6):410-3; Joseph et al., Pain. 2004 January; 107(1-2):147-58; Shin et al., Biochem Biophys Res Commun. Aug. 1, 2003; 307(3):696-703; Yamada et al., DNA Repair (Amst). Aug. 12, 2003; 2(8):909-24; Matsumi et al., AIDS. May. 23, 2003; 17(8):1127-37; Meng et al., Environ Mol Mutagen. 2002;39(4):282-95; Yao et al., Am J Physiol Cell Physiol. 2002 July; 283(1):C155-68; Krown et al., J Interferon Cytokine Res. 2002 March; 22(3):295-303; Torres, GMHC Treat Issues. 1995 January; 9(1):7-9; Boonprakong, STEP Perspect. 1997 Winter. 9(1):9-12; Santiago, GMHC Treat Issues. 1996 February; 10(2):9-11; Sanne, Int J Infect Dis. 2001;5(1):43-8; Roth et al., J Mass Spectrom. 2000 November; 35(11):1313-9; Imamichi et al., J Virol. 2000 December; 74(23):10958-64; Meng et al., Proc Natl Acad Sci U S A. Nov. 7, 2000; 97(23):12667-71; Lipsitz et al., Biometrics. 2000 June; 56(2):528-36; Walters et al., J Pharm Biomed Anal. 1999 May; 19(6):955-65; Davidson et al., Am J Med. Apr. 15, 2004; 116(8):546-54; Turtureanu-Hanganu, Rev Med Chir Soc Med Nat Iasi. 2002 October-December; 106(4):705-11; Holdsworth et al., Am J Health Syst Pharm. Nov. 1, 2003; 60(21):2213-22; quiz 2223-4; Coiffier et al., J Clin Oncol. Dec. 1, 2003; 21(23):4402-6; Seki et al., Ann Pharmacother. 2003 May; 37(5):675-8; Lee et al., Ann Pharmacother. 2003 November; 37(11):1614-7; Goldman, Expert Rev Anticancer Ther. 2003 August; 3(4):429-33; Ribeiro et al., Clin Lymphoma 2003 March; 3(4):225-32; Navolanic et al., Leukemia 2003 March; 17(3):499-514; Hussain et al., Am J Hematol. 2003 March; 72(3):212-5; Vaisban et al., Am J Med Sci. 2003 January; 325(1):38-40; Bosly et al., Cancer. Sep. 1, 2003; 98(5):1048-54; Kopecna et al., Bratisl Lek Listy. 2002;103(6):206-9; Baeksgaard et al., Cancer Chemother Pharmacol. 2003 March; 51(3):187-92; Lerza et al., Leuk Lymphoma. 2002 May; 43(5):1129-32; Liberopoulos et al., Ann Clin Lab Sci. 2002 Fall; 32(4):419-21; Cairo, Clin Lymphoma. 2002 December; 3 Suppl 1:S26-31; Cabanillas, Clin Lymphoma. 2002 December; 3 Suppl 1:S32-6; Yim et al., Ann Pharmacother. 2003 July-August; 37(7-8):1047-54; Kaplow R., Clin J Oncol Nurs. 2002 March-April; 6(2):110; Levine, Clin Lymphoma 2002 December; 3 Suppl 1:S19-25; Pui, Expert Opin Pharmacother. 2002 April; 3(4):433-42; Inai et al., Nippon Rinsho. 2003 January; 61 Suppl 1:299-303; K. G. Cunningham et al., J. Chem. Soc., 2299 (1951); N. M. Kredich and A. J. Guarino, Biochim. Biophys. Acta, 41:363 (1960); H. R. Bentley et al., J. Chem. Soc., 2301 (1951); E. A. Kaczka et al., Biochem. Biophys. Res. Commun., 14, 456 (1964); R. Suhadolnik et al., J. Am. Chem. Soc., 86:948 (1964); A. R. Todd and T. L. Ulbricht, J. Chem. Soc., 1960:3275; W. W. Lee et al., J. Am. Chem. Soc., 83:1906 (1961); E. Walton et al., J. Am. Chem. Soc., 86:2952 (1964); Y. Ito et al., J. Am. Chem. Soc., 103:6739 (1981); H. T. Shigeura and G. E. Boxer, Biochem. Biophys. Res. Commun., 17:758 (1964); S. Penman et al., Proc. Nat. Acad. Sci. USA, 67, 1878 (1970); J. J. Fox. et al., Progr. Nucleic Acid Res. Mol. Biol., 5:258-262 (1966); A. J. Guarino, “Cordycepin” in Antibiotics I, D. Gottlieb, P. Shaw, Eds. (Springer-Verlag, New York, 1967) pp 468-480; H. Klenow, Biochim. Biophys. Acta, 76:347 (1963); H. Shigeura and S. Sampson, Biochim. Biophys. Acta, 138:26 (1967), J. J. Novak and F. Sorm, Coll. Czech. Chem. Commun., 38:113 (1973); M. Blandin, J. Carbohyd. Nucl., Nucl., 3(5/6):341 (1976); Hendrickson et al., Nucleic Acids Res. 2004;32(7):2241-50; Wang et al., Nucleosides Nucleotides Nucleic Acids. 2004;23(1-2):161-70; Gouder et al., J Neurosci. Jan. 21, 2004; 24(3):692-701; Singh et al., J Enzyme Inhib Med Chem. 2003 October; 18(5):395-402; Tkachenko et al., J Cell Sci. Jul. 1, 2004; 117(Pt 15):3189-99; Ekbom et al., Blood Press. 2004;13(3):137-141; Deruelle et al., Eur J Obstet Gynecol Reprod Biol. Jul. 15, 2004; 115(1):106-7; Li et al., J Appl Physiol. Jun. 25, 2004; Zhou et al., J Med Chem. Jan. 29, 2004; 47(3):566-75; Doláková et al., Nucleosides Nucleotides Nucleic Acids. 2003 December; 22(12):2145-60; Whitfield et al., Chem Commun (Camb). Nov. 21, 2003(22):2802-3; Okuyama et al., Biosci Biotechnol Biochem. 2003 May; 67(5):989-95; Lecoq, Int. Z. Vitaminforsch. 27:291 (1957); Kossel, Ber., 18:79, 1928 (1885). Fischer, Ber, 30:2226 (1897); Traube, Ann., 331:64 (1904); Hoffer, Jubilee Vol. Emil Barell, 428-434 (1946); Taylor et al., Ciba Foundation Symposium, Chem. and Biol. Purines, 1957, 20, C.A. 53, 6238b (1959); Bredereck et al., Anew. Chem., 71:524 (1959); Morita et al., Chem. & Ind. (London) 1117 (1968); Sekiya, Suzuki, Chem. Pharm. Bull., 20:209 (1972); N. J. Kos et al., J. Org. Chem., 44:3140 (1979); Philips et al., J. Pharmacol. Exp. Ther., 104:20 (1952); Ts'o, “Bases, Nucleosides and Nucleotides” in Basic Principles in Nucleic Acid Chemistry vol. 1, P.O.P. Ts'o, Ed. (Academic Press, New York, 1974) pp 453-584; U.S. Pat. No. 6,646,125; U.S. Pat. No. 6,566,365; U.S. Pat. No. 6,545,001; U.S. Pat. No. 6,511,983; U.S. Pat. No. 6,358,963; U.S. Pat. No. 6,175,008; U.S. Pat. No. 5,466,806; U.S. Pat. No. 5,994,321; U.S. Pat. No. 5,912,356; U.S. Pat. No. 5,869,467; U.S. Pat. No. 5,840,990; U.S. Pat. No. 5,808,147; U.S. Pat. No. 5,641,889; U.S. Pat. No. 5,684,164; U.S. Pat. No. 5,869,467; U.S. Pat. No. 5,446,045; U.S. Pat. No. 5,955,610; U.S. Pat. No. 4,968,674; U.S. Pat. No. 4,283,394; U.S. Pat. No. 4,138,562; U.S. Pat. No. 6,472,507; U.S. Pat. No. 5,118,672; U.S. Pat. No. 6,677,310; U.S. Pat. No. 6,472,507; U.S. Pat. No. 6,579,857; U.S. Pat. No. 5,756,706; U.S. Pat. No. 5,744,596; U.S. Pat. No. 5,654,286; U.S. Pat. No. 5,541,164; U.S. Pat. No. 5,506,213; U.S. Pat. No. 5,310,732; US2004043955; EP0963997; U.S. Pat. No. 6,436,947; U.S. Pat. No. 4,283,394; and U.S. Pat. No. 4,138,562.

More specifically, suitable adenosine nucleotides, adenosine nucleosides and adenosine bases include antiviral, antimetabolite (antimetabolites by definition are substances that interfere with the body's chemical processes, such as creating proteins, DNA, and other chemicals needed for cell growth and reproduction or disrupt DNA production preventing cell division) or other agents.

Suitable antiviral agents include (1S,4R)-4-[2-Amino-6-(cyclopropylamino)-9H-purin-9-yl]-2-cyclopentene-1-methanol; (−)-cis-4-[2-amino-6-(cyclopropylamino)-9H-purin-9-yl]-2-cyclopentene-1-methanol; cis-2-hydroxymethyl-4-(2-amino-6-cyclobutylamino-purine-9-yl)-1,3-dioxolane ((2S,4S)-4-(2-amino-6-(cyclobutylamino)-9H-purin-9-yl)-1,3-dioxolan-2-yl)methanol; cis-2-hydroxymethyl-4-(2-amino-6-cyclopentylamino-purine-9-yl)-1,3-dioxolane ((2S,4S)-4-(2-amino-6-(cyclopentylamino)-9H-purin-9-yl)-1,3-dioxolan-2-yl)methanol; cis-2-hydroxymethyl-4-(2-amino-6-diamino-purine-9-yl)-1,3dioxolane ((2S,4S)-4-(2,6,8-triamino-9H-purin-9-yl)-1,3-dioxolan-2-yl)methanol; 2-amino-9-((2R,3R,4S,5R)-tetrahydro-3,4-dihydroxy-5-(hydroxymethyl)furan-2-yl)-1H-purin-6(9H)-one; cis-2-hydroxymethyl-4-(2-amino-6-chloro-purine-9-yl)-1,3-dioxolane ((2S,4S)-4-(2-amino-6-chloro-9H-purin-9-yl)-1,3-dioxolan-2-yl)methanol; [[(1R)-2-(6-Amino-9H-purin-9-yl)-1-methylethoxy]methyl]phosphonic acid; (R)-9-(2-phosphonomethoxypropyl)adenine; (R)-PMPA;: 5-[[(1R)-2-(6-Amino-9H-purin-9-yl)-1-methylethoxy]methyl]-2,4,6,8-tetraoxa-5-phosphanononanedioic acid bis(1-methylethyl) ester 5-oxide; (R)-bis(POC)PMPA; 2′,3′-Dideoxyinosine; dideoxyinosine; ddI; ddIno; 1H-Purin-6-amine; 6-aminopurine; 6-amino-1H-purine; 9-beta-D-Arabino furanosyl adenine; 9-Arabinosyladenine; 9-beta-D-Arabino furanosyl adenine; 9-beta-D-arabinofuranosyladenine monohydrate; 9H-Purin-6-amine, 9-beta-D-arabinofuranosyl-, monohydrate; Adenine, 9-beta-D-arabinofuranosyl- (8CI); Adenine arabinoside; Ara-A; Araadenosine; Arabinosyladenine; Arasena-A; beta-D-Arabinosyladenine; Spongoadenosine; 6-amino-3H-purine; 6-amino-9H-purine; 2-Amino-1,9-dihydro-9-((2-hydroxyethoxy)methyl)-6H-purin-6-one; 2-amino-1,9-dihydro-9-[2-hydroxyethoxy-methyl]-purin-6-one; 9-((2-hydroxyethoxy)methyl)guanine; Aciclovir; 2-Amino-1,9-dihydro-9-((2-hydroxy-1-(hydroxymethyl)ethoxy)methyl)-6H-purin-6-one; 2′-NDG; 2′-nor-2′-deoxyguanosine; 9-(1,3-Dihydroxy-2-propoxymethyl)guanine; 9-((2-Hydroxy-1-(hydroxymethyl)ethoxy)-methyl)guanine; Cymevan; Cymevene; Cytovene; 1-beta-D-Ribofuranosyl-1,2,4-triazole-3-arboxamide; 1-beta-D-ribofuranosyl-1H-1,2,4-triazole-3-carboxamide; icn-1229; DHPG; Acycloguanosine; Acyclo-V; 1,6-dihydro-6-iminopurine; (2R,4R)-4-(2,6-diamino-9H-purin-9-yl)-1,3-Dioxolane-2-methanol; 2,6 Diaminopurine dioxolane; (2R-cis)-4-(2,6-Diamino-9H-purin-9-yl)-1,3-dioxolane-2-methanol; 3,6-dihydro-6-iminopurine; and adenine.

Suitable antiviral agents also include Abacavir; Ziagen (Glaxo Wellcome); Tenofovir; Tenofovir disoproxil; Tenofovir DF; Didanosine; Vidarabine; Vidarabina; Vira-A; Acyclovir; DAPD (Amdoxovir; is a guanine analog from Triangle, which is converted in vivo to the highly potent DXG); BW-248U; Vipral; Virorax; Wellcome-248U; Zovirax; Zyclir; Famciclovir; Penciclovir; Valacyclovir; Ganciclovir; Ganciclovir sodium; Vitrasert; Ribavarin; RTCA; Tribavirin; Vilona; Viramid; Virazid; Virazole; Valacyclovir (Valacyclovir is the hydrochloride salt of 1-valyl ester of acyclovir); DAPD; Abacavir; Abacavir succinate; Ziagen; Famciclovir; Famvir and Videx (Bristol-Myers Squibb).

Additional suitable antiviral agents include those disclosed, e.g., in EP 349242; U.S. Pat. No. 5,034,394; M. T. Crimmins, and B. W. King, J. Org. Chem., 61:4192 (1996); S. M. Daluge et al., Antimicrob. Ag. Chemother., 41:1082 (1997); R. H. Foster and D. Faulds, Drugs 55:729-736 (1998); S. Staszewski et al., J. Am. Med. Assoc., 285:1155 (2001); Rosenberg et al., Coll. Czech. Chem. Commun., 53:2753 (1988); A. Holy et al., Coll. Czech. Chem. Commun., 60:1390 (1995); L. M. Schultze et al., Tetrahedron Letters, 39:1853 (1998); M. N. Arimilli et al., Antivir. Chem. Chemother., 8:557 (1997); WO 98 04569; U.S. Pat. No. 5,922,695; J. Balzarini et al., Biochem. Biophys. Res. Commun., 219:337 (1996); J.-P. Shaw et al., Pharm. Res., 14:1824 (1997); S. G. Deeks et al., Antimicrob. Ag. Chemother., 42:2380 (1998); W. Plunkett and S. S. Cohen, Cancer Res. 35:1547 (1975); EP 206497; R. R. Webb et al., Nucleosides Nucleotides, 7:147 (1988); H. Mitsuya and S. Broder, Proc. Nat. Acad. Sci. USA, 83:1911 (1986); U.S. Pat. No. 5,808,147; D. D. Richman, Antimicrob. Ag. Chemother., 31:1879 (1987); J. E. Dahlberg et al., Proc. Nat. Acad. Sci. USA, 84:2469 (1987); G. Ahluwalia et al., Biochem. Pharmacol., 36:3797 (1987); G. Ray and E. Murrill, Anal. Letters, 20, 1815 (1987); J. S. Lambert et al., N. Engl. J. Med., 322:1333 (1990); T. P. Cooley et al., N. Engl. J. Med. 322:1340. M. N. Nassar et al., Anal. Profiles Drug Subs. Excip., 22:185-227 (1993); Lecoq, Int. Z. Vitaminforsch., 27:291 (1957); U.S. Pat. No. 6,358,963; Kossel, Ber. 18:79, 1928 (1885). Fischer, Ber., 30:2226 (1897); Traube, Ann., 331:64 (1904); Hoffer, Jubilee Vol. Emil Barell, 428-434 (1946); Taylor et al., Ciba Foundation Symposium, Chem. and Biol. Purines, 1957, 20, C.A. 53, 6238b (1959); U.S. Pat. No. 6,509,320; Bredereck et al., Angew. Chem., 71:524 (1959); Morita et al., Chem. & Ind. (London) 1117 (1968); Sekiya, Suzuki, Chem. Pharm. Bull., 20:209 (1972); N. J. Kos et al., J. Org. Chem., 44:3140 (1979); Philips et al., J. Pharmacol. Exp. Ther., 104:20 (1952); Kamel et al., Eur J Mass Spectrom (Chichester Eng). 2004; 10(2):239-57; Lee, Drugs Today (Barc). 1998 March; 34(3):241-9; Wilhelmus, Cochrane Database Syst Rev. 2003 (3):CD002898; Shiraki, Nippon Rinsho. 2003 February; 61 Suppl 2:792-7; Niwa et al., Oncol Rep. 2003 September-October; 10(5):1437-41; Hayashi et al., Histol Histopathol. 2003 October; 18(4): 1155-68; Spiers, Haematologia (Budap). 1996;27(2):55-84; U.S. Pat. No. 6,677,310; Henrot, Ann Dermatol Venereol. 2002 April; 129(4 Pt 2):533-49; Worthington, N Engl J Med. May 8, 2003; 348(19):1920-5; author reply 1920-5; Chilukuri et al., Dermatol Clin. 2003 April; 21(2):311-20; Kleymann, Expert Opin Investig Drugs. 2003 February; 12(2):165-83; Klammer et al., Hautarzt. 2003 April; 54(4):362-4; Kiewe et al., Leuk Lvmphoma. 2003 October; 44(10):1815-8; Junghanss et al., Br J Haematol. 2003 November;123(4):662-70; Chosidow et al., Ann Dermatol Venereol. 2002 April; 129(4 Pt 2):635-45; Ishii et al., Intern Med. 2003 March; 42(3):250-4; Holdiness, Contact Dermatitis. 2001 May; 44(5):265-9; Wilhelmus, Trans Am Ophthalmol Soc. 2000;98:505-32; Wilhelmus, Cochrane Database Syst Rev. 2001 (1):CD002898; Josephson et al., Antivir Ther. 1996 August; 1(3):147-56; Spiers, Am J Ther. 1995 March; 2(3):196-216; Wang et al., Zhonghua Shi Yan He Lin Chuang Bing Du Xue Za Zhi. 1998 March; 12(1):12-4; Thomas-Golbanov et al., Expert Opin Investig Drugs. 2001 July; 10(7):1279-89; Hernández et al., Biopolymers. 2002;67(6):440-55; Morales et al., Am J Ophthalmol. 2001 July; 132(1):114-6; Field et al., Eur J Haematol. 2002 July; 69(1):54-7; Fraternale et al., Eur J Clin Invest. 2001 March; 31(3):248-52; Rossi et al., Br J Haematol. 2001 October; 115(1):58-62; Bordigoni et al., Clin Infect Dis. May 1, 2001; 32(9):1290-7; Kesson, Paediatr Drugs. 2001; 3(2):81-90; Kimura et al., J Pediatr Hematol Oncol. 2001 June-July; 23(5):294-9; Tran et al., Gastroenterol Clin Biol. 2000 December; 24(12):1234-5; Kamiyama et al., J Gen Virol. 2001 November; 82(Pt 11):2761-5; Fried et al., Antivir Ther. 1996 April; 1(2):71-6; Aoki, Herpes. 2001 July; 8(2):41-5; Hambach et al., Bone Marrow Transplant. 2001 October; 28(7):705-7; Su et al., Zhonghua Shi Yan He Lin Chuang Bing Du Xue Za Zhi. 2002 March; 16(1):16-9; Abruzzo et al., Am J Surg Pathol. 2002 May; 26(5):630-6; Magnani et al., J Leukoc Biol. 2003 November; 74(5):764-71; Jindra et al., Bone Marrow Transplant. 2003 May; 31(10):951-2; Stratta et al., Transplant Proc. 2004 May; 36(4):1082-3; Chi-Ming Chiu et al., J Ethnopharmacol. 2004 July; 93(1):63-8; Labetoull, J Fr Ophtalmol. 2004 May; 27(5):547-57; Hurwitz et al., Pediatr Transplant. 2004 June; 8(3):267-72; Andrei et al., Antiviral Res. 2004 March; 61(3):181-7; Clark et al., Transplantation. May 15, 2004; 77(9):1480; Whitley, Curr Opin Infect Dis. 2004 June; 17(3):243-6; Genova et al., Toxicol Appl Pharmacol. Jun. 1, 2004; 197(2):107-12; Cheshenko et al., Antimicrob Agents Chemother. 2004 June; 48(6):2025-36; Benarroch et al., J Biol Chem. May 19, 2004; De Clercq et al., Nucleosides Nucleotides Nucleic Acids. 2001 April-July; 20(4-7):271-85; De Clercq, Nucleosides Nucleotides Nucleic Acids. 2000 October-December; 19(10-12):1531-41; De Clercq et al., Rev Med Virol. 2001 November-December; 11(6):381-95; Berenguer et al., Proc Assoc Am Physicians. 1998 March-April; 110(2):98-112; Andrei et al., Antiviral Res. 2004 March; 61(3):181-7; De Clercq, J Clin Virol. 2004 June; 30(2):115-33; Maggs et al., Am J Vet Res. 2004 April; 65(4):399-403; Kim et al., Org Biomol Chem. Apr. 21, 2004; 2(8):1164-8; Satoh et al., Int J Radiat Oncol Biol Phys. Jun. 1, 2004; 59(2):562-71; Walling et al., J Infect Dis. May 1, 2004; 189(9):1656-63; Brodell et al., Postgrad Med. 2004 April; 115(4):63-5; Lee, Drugs Today (Barc). 1998 March; 34(3):241-9; Morin et al., Nucl Med Biol. 2004 July; 31(5):623-30; Bidanset et al., Antiviral Res. 2004 July; 63(1):61-4; Stratta et al., Transplant Proc. 2004 May; 36(4):1082-3; Hurwitz et al., Pediatr Transplant. 2004 June; 8(3):267-72; Maggon et al., Rev Med Virol. 2004 May-June; 14(3):149-68; Medeiros et al., J Clin Gastroenterol. 2004 January; 38(1):84-6; Ribeiro et al., Hepatology. 2003 August; 38(2):509-17; Romero-Gómez et al., Am J Gastroenterol. 2003 July; 98(7):1621-6; Seignères et al., Antimicrob Agents Chemother. 2003 June; 47(6):1842-52; Chong et al., Antivir Chem Chemother. 2002 March; 13(2):115-28; Seignères et al., Hepatology. 2002 September; 36(3):710-22; Pramoolsinsup, J Gastroenterol Hepatol. 2002 February; 17 Suppl:S125-45; Sumpter et al., Antiviral Res. 2004 June; 62(3):111-20; De Clercq, J Clin Virol. 2004 June; 30(2):115-33, MacManus et al., AIDS. Mar. 5, 2004; 18(4):651-5; Blanckenberg et al., AIDS. Mar. 5, 2004; 18(4):631-40; Ts'o, “Bases, Nucleosides and Nucleotides” in Basic Principles in Nucleic Acid Chemistry vol. 1, P.O.P. Ts'o, Ed. (Academic Press, New York, 1974) pp 453-584; U.S. Pat. No. 6,646,125; U.S. Pat. No. 6,566,365; U.S. Pat. No. 6,545,001; U.S. Pat. No. 6,511,983; U.S. Pat. No. 6,358,963; U.S. Pat. No. 6,175,008; U.S. Pat. No. 5,466,806; U.S. Pat. No. 5,994,321; U.S. Pat. No. 5,912,356; U.S. Pat. No. 5,869,467; U.S. Pat. No. 5,840,990; U.S. Pat. No. 5,808,147; U.S. Pat. No. 5,641,889; U.S. Pat. No. 5,684,164; U.S. Pat. No. 5,869,467; U.S. Pat. No. 5,446,045; U.S. Pat. No. 5,955,610; U.S. Pat. No. 4,968,674; U.S. Pat. No. 4,283,394; U.S. Pat. No. 4,138,562; U.S. Pat. No. 6,472,507; U.S. Pat. No. 5,118,672; and U.S. Pat. No. 6,677,310.

Suitable antimetabolite agents include, e.g., 9-β-D-Arabinofuranosyl-2-fluoro-9H-purin-6-amine; 9-β-D-arabinofuranosyl-2-fluoroadenine; 2-fluorovidarabine; 2-fluoro-9-β-D-arabinofuranosyladenine; 6-(1,3-Dihydro-7-hydroxy-5-methoxy-4-methyl-1-oxoisobenzofuran-6-yl)-4-methyl-4-hexanoic acid; 2-Fluoroadenine arabinoside; 2-Fluoro Ara-A; 2-chloro-2′-deoxyadenosine; 1,7-dihydro-6H-purine-6-thione; 3H-Purine-6-thiol; 6-Mercaptopurine; 6 MP; 6-Purinethiol; 6-Thioxopurine; 7-Mercapto-1,3,4,6-tetrazaindene; Hypoxanthine, thio; Ismipur; Leukeran; Leukerin; Leukerin, 99%—Carc.; Leupurin; Mercaleukim; Mercaleukin; Mercaptopurine; Mercapurin; Mern; Purimethol; Purine-6-thiol; Purinethiol; Puri-Nethol; U-4748; 2-F-araA; 2-amino-1,7-dihydro-6H-purine-6-thione; 2-Amino-6-mercaptopurine; 2-Amino 6MP; 2-Amino-6-purinethiol; 2-Aminopurine-6(1H)-thione; 2-Aminopurine-6-thiol; 6-Mercapto-2-aminopurine; 6-Mercaptoguanine; 6-TG; bw 5071; Lanvis; 6-[(1-methyl-4-nitro-1H-imidazol-5-yl)thio]-1H-Purine; 6-(1-methyl-p-nitro-5-imidazolyl)-thiopurine; 6-((1-methyl-4-nitroimidazol-5-yl)thio)purine; 6-(methyl-p-nitro-5-imidazolyl)-thiopurine; Azathioprine; azatioprin; azothioprine; bw 57-322; ccucol; Cytostatics; Imuran; imurek; imurel; methylnitroimidazolylmercaptopurine; Tabloid; ',3′-dideoxyinosine; Didanosine; Dideoxyinosine; TG; THG; Thioguanine; 1,5-Dihydro-4H-pyrazolo(3,4-d)pyrimidin-4-one; 1H-Pyrazolo(3,4-d)pyrimidin-4-ol; Bleminol; Bloxanth; bw 56-158; Caplenal; Capurate; Cellidrin; Cosuric; dabrosin; Dabroson; dura AL; Embarin; Epidropal; Foligan; Geapur, Gichtex; Gotax; Hamarin; Hexanurat; HHP; HPP; Ketanrift; Ketobun-A; Ledopur; Lopurin; Lysuron; milurit; Miniplanor, Monarch; Nektrohan; Progout; Remid; Riball; Sigapurol; Suspendol; Takanarumin; Urbol; Uricemil; uriprim; Uripurinol; uritas; Urobenyl; Urosin; Urtias; Urtias 100; Xanturat; Zygout; Zyloprim; Zyloric; 4H-pyrazolo(3,4-d)pyrimidin-4-one; 4-hydroxy-1H-pyrazolo(3,4-d)pyrimidine; 4-hydroxy-3,4-pyrazolopyrimidine; 4-hydroxypyrazolo[3,4-d]pyrimidine; 4′-hydroxypyrazolo[3,4-d]pyrimidine; 4-hydroxypyrazolopyrimidine; Adenock; Al-100; allopur; Allo-puren; Allopurinol; allopurinol(I); Allorin; Allozym; Allural; Aloral; Alositol; Aluline; Anoprolin; Anzief; Apulonga; Apurin; Apurol; atisuril; Tioguanine; Wellcome U3B; Thioguanine and 2-F-ara-AMP.

Additional suitable antimetabolite agents include, e.g., fludarabine (F-ara-A); mycophenolic Acid; cladribine (CdA, 2-CdA, Leustatin®, 2-chlorodeoxyadenosine, 2-chloro-2′-deoxy-β-D-adenosine, 2-chloro-2′-deoxyadenosine, NSC-105014-F, 2-chloro-6-amino-9-(2-deoxy-β-D-erythropento-furanosyl) purine); clofarabine (Cl-F-ara-A); Ara-A; Ara-G (guanosine arabinoside); nelarabine (506U78); clofarabine (CAFdA); Ara-C (cytosine arabinoside or cytarabine); gemcitabine (dFdC); bendamustine; 6-Mercaptopurine; 5-fluorodeoxyuridine; 5-fluorouridine; 6-azauridine; 2-halo-2′-deoxyadenosine (one of which is 2-chlorodeoxyadenosine); 2-arabino-chloro-2′-fluoroaradeoxyadenosine; 9-(β-D-arabinofuranosyl)-2-fluoroadenine; 6-methylmercaptopurine riboside; dideoxycytidine; dideoxythymidine; dideoxyguanosine; dideoxyinosine; dideoxyadenosine; 2′-deoxytubercidin; 2′-deoxy-(3,4-d)pyrimidine; acyclovir; ganciclovir; 8-chloroadenosine; 2′-azaido-2′-deoxyuridine and 2′-azido-2′-deoxycytidine (or more broadly, azidonucleosides); immucillin-H; thioguanine; videx; azathioprine; dideoxyinosine; azathioprine; and allopurinol.

Additional suitable antimetabolite agents include those disclosed, e.g., in J. A. Montgomery and K. Hewson, J. Med. Chem., 12, 498 (1969); J. A. Montgomery et al., J. Heterocycl. Chem., 16:157 (1979); U.S. Pat. No. 4,210,745 R. W. Brockman et al., Biochem. Pharmacol., 26:2193 (1977);. W. Brockman et al., Cancer Res., 40:3610 (1980); V. Gandhi et al., J. Chromatog., 413:293 (1987); U.S. Pat. No. 4,357,324; M. R. Hersh et al., Cancer Chemother. Pharmacol., 17:277 (1986); Eibschutz B. et al., Arthritis & Rheumatism, 38(11):1604-1609 (1995); U.S. Pat. No. 5,541,164; Zinzani, P. L. et al., American Journal of Hematology, 47:301-306 (1994); Ilyas, W. et al., J Am Acad Dermatol., 41(2):316-318; R. P. Warrell, Jr. and E. Berman, J. Clin. Oncol., 4:74 (1986); H. G. Chun et al., Cancer Treat. Rep. 70:1225 (1986); Levine et al., Biol Blood Marrow Transplant., 9(3):189-97 (2003); Feinstein et al., Biol Blood Marrow Transplant., 9(4):266-72 (2003); Maris et al., Blood, 102(6):2021-30 (2003); Georges et al., Int J Hematol., 76 Suppl 1:184-9 (2002); Murotani et al., Clin Lab Haematol., 24(5):303-6 (2002); U.S. Pat. No. 4,088,756; U.S. Pat. No. 5,541,164; U.S. Pat. No. 5,654,286; U.S. Pat. No. 6,309,663; U.S. Pat. No. 6,720,001; Mielcarek et al., Blood 102(2):756-62 (2003); Maris et al., Transfus Clin Biol., 8(3):231-4 (2001); Niederwieser et al., Blood 101(4):1620-9 (2003); Kreiter et al., Bone Marrow Transplant., 28(2):157-61 (2001); Kolesar, J. M. et al., J Oncol Pharm Practice, 2(3):160-181 (1996); Kolesar, J. M. et al., J Oncol Pharm Practice, 2(4):211-224 (1996); Morris, A. K., et al., J Oncol Pharm Practice, 3(2):94-109 (1997); Feinstein et al., Ann N Y Acad Sci., 938:328-37; discussion 337-9 (2001); Ruiz-Argüelles, Rev Invest Clin., 54(2):154-60 (2002); Tsimberidou et al., Cancer. 100(12):2583-91 (2004); Ferrajoli et al., Semin Oncol., 31(2 Suppl 4):60-5 (2004); Zent et al., Leuk Res., 28(5):495-507 (2004); de Leval et al., Am J Surg Pathol., 28(5):679-86 (2004); Hillmen, Semin Oncol., 31(1 Suppl 2):22-6 (2004); Tam et al., Cancer, 100(10):2181-9 (2004); Huynh et al., Clin Nucl Med., 29(4):275-6 (2004); Rossi et al., J Clin Oncol., 22(7):1260-7 (2004); Nabhan et al., Leuk Res. , 5:429-42 (2004); Shvidel et al., Leuk Lymphoma, 44(11):1947-50 (2003); Erba H P., Curr Hematol Rep., (1):47-53 (2004); Lozanski et al., Blood, 103(9):3278-81 (2004); Tóthová et al., Neoplasma, 50(6):433-7 (2003); Hallek et al., MMW Fortschr Med., 145(27-28):46-9 (2003); Friedberg et al., Leuk Res., 28(2):139-47 (2004); Jones et al., Blood, 103(5):1855-61 (2004); Imagawa et al., Gan To Kagaku Ryoho, 31(2):267-70 (2004); Usman et al., J Pak Med Assoc., 53(10):496-7 (2003); Mamani-Matsuda et al., Br J Haematol., 124(2):141-50 (2004); Kiewe et al., Leuk Lymphoma, 44(10):1815-8 (2003); Braester et al., Leuk Lymphoma, 44(10):1835-6 (2003); Montillo et al., Leukemia, 18(1):57-62 (2004); Tiscornia et al., Leukemia, 18(1):48-56 (2004); Stiefelhagen, MMW Fortschr Med., 145(39):55-6 (2003); Frankel et al., Clin Cancer Res., 9(10 Pt 1):3555-61 (2003); Pahler et al., Clin Cancer Res. 9(12):4570-7 (2003); Schetelig et al., J Clin Oncol., 21(14):2747-53 (2003); Johnston et al., Oncogene, 22(51):8356-69 (2003); Schiavone et al., Eur J Haematol., 71(1):23-8 (2003); Giles et al., Cancer Chemother Pharmacol., 52(3):223-8 (2003); Pettitt, Br J Haematol., 121(5):692-702 (2003); Castagna et al., Blood, 102(6):2309; author reply 2309-10 (2003); Starczynski et al., Br J Haematol., 123(1):66-71 (2003); Dreger et al., Leukemia, 17(5):841-8 (2003); Wells et al., Ann Pharmacother., 37(5):671-4 (2003); Tournilhac et al., Blood, 103(1):363-5 (2004); Weiss et al., J Clin Oncol., 21(7):1278-84 (2003); Montserrat, Semin Oncol. 2003 February; 30(1 Suppl 2):34-9; Morabito et al., Haematologica 2003 August; 88(8):864-73; Beyan et al., Haematologia (Budap). 2002; 32(3):287-90; Rubnitz et al., Leuk Res. 2004 April; 28(4):349-52; Holowiecki et al., Leukemia. 2004 May; 18(5):989-97; Juliusson et al., Br J Haematol. 2003 December; 123(5):810-8; Wrzesień-Kuś et al., Eur J Haematol. 2003 September; 71(3):155-62; Robak, Leuk Lymphoma. 2003 March; 44(3):391-409; Zwaan et al., Blood. Nov. 1, 2002; 100(9):3352-60; Robak et al., Neoplasma. 2001; 48(3):203-7; Korycka et al., Leuk Lymphoma. 2003 September; 44(9):1549-55; Razzouk et al., Leukemia. 2001 September; 15(9):1326-30; Krance et al., J Clin Oncol. Jun. 1, 2001; 19(11):2804-11; Lindemalm et al,. Haematolozica. 2003 March; 88(3):324-32; Korycka et al., Arch Immunol Ther Exp (Warsz). 2003; 51(1):61-7; Crews et al., J Clin Oncol. Oct. 15, 2002; 20(20):4217-24; Seshadri, Leuk Res. 2000 July; 24(7):637; Robak et al., Leuk Lymphoma. 2000 September; 39(1-2):121-9; Lech-Marańda et al., Eur J Haematol. 2000 November; 65(5):317-21; Schirmer et al., Leuk Res. 1999 December; 23(12):1121-6; Gordon et al., Leuk Res. 2000 October; 24(10):871-5; Al-Fiar et al., Leuk Res. 1999 August; 23(8):719-21; Korycka et al., Arch Immunol Ther Exp (Warsz). 2000; 48(4):293-9; Niitsu et al., Leuk Res. 2000 January; 24(1):1-9; Van Den Neste et al., Ann Hematol. 1998 January; 76(1):19-23; Saito et al., Clin Cancer Res. 2002 April; 8(4):1014-20; Martinelli et al., Haematologica. 1996 May-June; 81(3):286-7; Giralt et al., Blood. Jun. 15, 1997; 89(12):4531-6; Korycka et al., Eur J Haematol. 2000 May; 64(5):347-9; Gollard et al., Leuk Lymphoma 1997 December; 28(1-2):183-5; Dann et al., J Clin Oncol. 1998 April; 16(4):1498-504; Aleskog et al., Eur J Haematol. 1999 May; 62(5):293-9; Korycka et al., Leuk Res. 1997 September; 21(9):857-65; Cheson et al., J Clin Oncol. 1999 August;17(8):2454-60; Kimby et al., Eur J Haematol. 2004 April; 72(4):292-5; Crews et al., J Pediatr Hematol Oncol. 2002 November; 24(8):677-80; Giralt et al., Blood. Feb. 1, 2001; 97(3):631-7; Mathew et al., Leukemia. 2000 July; 14(7):1314-5; Komblau et al., Leukemia. 1996 October; 10(10): 1563-9; Klippstein et al., J Cancer Res Clin Oncol. 2003 May,129(5):316-9; Konstantinov et al., J Cancer Res Clin Oncol. 2002 May; 128(5):271-8; Zulkowski et al., J Cancer Res Clin Oncol. 2002 February; 128(2):111-3; Ruffert, Zentralbl Chir. 1998; 123 Suppl 5:156-8; Balfour et al., Drugs. 2001;61(5):631-8; discussion 639-40; Brockmann et al., Arch Geschwulstforsch. 1989;59(5):341-6; Höffken et al., J Cancer Res Clin Oncol. 1998;124(11):627-32; Strumberg et al., Anticancer Drugs. 1996 June; 7(4):415-21; Mehta et al., Bone Marrow Transplant. 2004 June; 33(11):1107-14; Ishitsuka et al., Eur J Haematol. 2004 April; 72(4):280-4; Matsuda et al., Rinsho Ketsueki. 2003 September; 44(9):946-51; Chow et al., J Pediatr. 2004 May; 144(5):669-71; Toubai et al., Acta Haematol. 2003;109(4):199-201; Lobe et al., Leukemia. 2003 August; 17(8):16004; Carroll et al., Hematology (Am Soc Hematol Educ Program). 2003:102-31; Ma et al., Zhonghua Er Ke Za Zhi. 2003 December; 41(12):929-33; Stoneham et al., Br J Haematol. 2003 October:123(1):100-2; Lauten et al., Haematologica. 2003 November; 88(11):1253-8; Fenaux et al., Best Pract Res Clin Haematol. 2003 September; 16(3):495-501; Dervieux et al., Clin Pharmacol Ther. 2003 June; 73(6):506-16; Sanz et al., Best Pract Res Clin Haematol. 2003 September;L6(3):433-51; Nygaard et al., Clin Pharmacol Ther. 2004 April;75(4):274-81; Fehr et al., Ann Hematol. 2003 March; 82(3):193-6; Lennard et al., Br J Clin Pharmacol. 1993 October; 36(4):277-84; Efferth et al., Int J Oncol. 2003 August; 23(2):509-17; Schaeffeler et al., Leukemia 2003 July; 17(7):14224; Colović et al., Chemotherapy. 2003 June; 49(3):138-45; Starý et al., Cas Lek Cesk. 2003;142(7):404-9; Schmiegelow et al., J Clin Oncol. Apr. 1, 2003; 21(7):1332-9; Oliveira et al., Braz J Med Biol Res. 2004 May, 37(5):649-58; Halonen et al., Acta Paediatr. 2003;92(1):37-42; Bostrom et al., Blood. May 15, 2003; 101(10):3809-17; Krawczuk-Rybak et al., Endokrynol Diabetol Chor Przemiany Materii Wieku Rozw. 2003;9(1):35-40; Sastry et al., Pediatr Hematol Oncol. 2003 March; 20(2):157-60; Nygaard et al., Leukemia. 2003 July; 17(7):1344-8; Cheok et al., Nat Genet. 2003 May; 34(1):85-90; Harms et al., Blood. Oct. 15, 2003; 102(8):2736-40; Marshall E., Science. Oct. 24, 2003; 302(5645):588-90; De Abreu et al., Ann Clin Biochem. 2003 January; 40(Pt 1):70-4; Krawczuk-Rybak et al., Endokrynol Diabetol Chor Przemiany Materii Wieku Rozw. 2003;9(1):41-7; Ordentlich et al., J Biol Chem. Jul. 4, 2003; 278(27):24791-9; Martin et al., Pharmacotherapy. 2003 February; 23(2):260-4; Bessard et al., Therapie. 2004 January-February; 59(1):71-5; Benessahraoui et al., Arch Pediatr. 2003 October; 10(10):891-4; Rogalski et al., J Dermatol. 2003 January; 30(1):59-63; Soo et al., J Natl Cancer Inst. May 19, 2004; 96(10):796-800; Yamane et al., Biochem Biophys Res Commun. May 21, 2004; 318(1):297-302; Rooseboom et al., Pharmacol Rev. 2004 March; 56(1):53-102; Lebwohl et al., J Am Acad Dermatol. 2004 March; 50(3):416-30; Gunnarsdottir et al., Drug Metab Dispos. 2004 March; 32(3):321-7; Lange et al., J Clin Oncol. Jan. 1, 2004; 22(1):150-6; Bakshi et al., Indian J Pediatr. 2003 September; 70(9):755-8; Krynetski et al., Oncogene. Oct. 20, 2003; 22(47):7403-13; Wiernik et al., Leuk Lymphoma. 2003 September; 44(9):1515-21; Stoneham et al., Br J Haematol. 2003 October; 123(1):100-2; Heath et al., Pediatr Hematol Oncol. 2003 October-November; 20(7):497-504; Gamis et al., J Clin Oncol. Sep. 15, 2003; 21(18):3415-22; Vernole et al., Cancer Chemother Pharmacol. 2003 September; 52(3):185-92; Dervieux et al., Clin Pharmacol Ther. 2003 June; 73(6):506-16; Yan et al., Clin Cancer Res. 2003 June; 9(6):2327-34; Jess et al., Aliment Pharmacol Ther. Feb. 1, 2004; 19(3):287-93; Taylor et al., Eur J Neurol. 2004 February; 11(2):141; Gainon et al., Schweiz Rundsch Med Prax. Oct. 8, 2003; 92(41):1734-9; Balal et al., Transplant Proc. 2003 June; 35(4):1408-9; Maltzman et al., J Clin Invest. 2003 April; 111(8):1122-4; Rammert et al., Ther Umsch. 2003 March; 60(3):145-50; Alvarez Delgado et al., Gastroenterol Hepatol. 2003 January; 26(1):52-3; Wang et al., Clin Transplant. 2002 December; 16(6):410-3; Joseph et al., Pain. 2004 January; 107(1-2):147-58; Shin et al., Biochem Biophys Res Commun. Aug. 1, 2003; 307(3):696-703; Yamada et al., DNA Repair (Amst). Aug. 12, 2003; 2(8):909-24; Matsumi et al., AIDS. May 23, 2003; 17(8):1127-37; Meng et al., Environ Mol Mutagen. 2002;39(4):282-95; Yao et al., Am J Physiol Cell Physiol. 2002 July; 283(1):C155-68; Krown et al., J Interferon Cytokine Res. 2002 March; 22(3):295-303; Torres, GMHC Treat Issues. 1995 January; 9(1):7-9; Boonprakong, STEP Perspect. 1997 Winter; 9(1):9-12; Santiago, GMHC Treat Issues. 1996 February; 10(2):9-11; Sanne, Int J Infect Dis. 2001;5(1):43-8; Roth et al., J Mass Spectrom. 2000 November; 35(11):1313-9; Imamichi et al., J Virol. 2000 December; 74(23):10958-64; Meng et al., Proc Natl Acad Sci U S A. Nov. 7, 2000; 97(23):12667-71; Lipsitz et al., Biometrics. 2000 June; 56(2):528-36; Walters et al., J Pharm Biomed Anal. 1999 May,L9(6):955-65; Davidson et al., Am J Med. Apr. 15, 2004; 116(8):546-54; Turtureanu-Hanganu, Rev Med Chir Soc Med Nat Iasi. 2002 October-December; 106(4):705-11; Holdsworth et al., Am J Health Syst Pharm. Nov. 1, 2003; 60(21):2213-22; quiz 22234; Coiffier et al., J Clin Oncol. Dec. 1, 2003; 21(23):4402-6; Seki et al., Ann Pharmacother. 2003 May; 37(5):675-8; Lee et al., Ann Pharmacother. 2003 November; 37(11):1614-7; Goldman, Expert Rev Anticancer Ther. 2003 August; 3(4):429-33; Ribeiro et al., Clin Lymphoma. 2003 March; 3(4):225-32; Navolanic et al., Leukemia. 2003 March; 17(3):499-514; Hussain et al., Am J Hematol. 2003 March; 72(3):212-5; Vaisban et al., Am J Med Sci. 2003 January; 325(1):38-40; Bosly et al., Cancer. Sep. 1, 2003; 98(5):1048-54; Kopecna et al., Bratisl Lek Listy. 2002;103(6):206-9; Baeksgaard et al., Cancer Chemother Pharmacol. 2003 March; 51(3):187-92; Lerza et al., Leuk Lymphoma. 2002 May; 43(5):1129-32; Liberopoulos et al., Ann Clin Lab Sci. 2002 Fall; 32(4):419-21; Cairo, Clin Lymphoma. 2002 December; 3 Suppl 1:S26-31; Cabanillas, Clin Lymphoma. 2002 December; 3 Suppl 1:S32-6; Yim et al., Ann Pharmacother. 2003 July-August; 37(7-8):1047-54; Kaplow R., Clin J Oncol Nurs. 2002 March-April; 6(2): 110; Levine, Clin Lymphoma. 2002 December; 3 Suppl 1:S19-25; Pui, Expert Opin Pharmacother. 2002 April; 3(4):433-42; Inai et al., Nippon Rinsho. 2003 January; 61 Suppl 1:299-303; and Sem. Oncol., 17:Suppl. 8, 1-78 (1990).

Other suitable agents include those disclosed in Levene and Bass, Nucleic Acids, p. 163 (New York, 1931); Levene, Tipson, J. Biol. Chem., 94:809 (1932); Bredereck, Ber., 66, 198 (1933); Z. Physiol. Chem., 223:61 (1934); U.S. Pat. No. 6,677,310; Gulland, Holiday, J. Chem. Soc., 765 (1936); U.S. Pat. No. 6,734,178; Szent-Györgyi, J. Physiol., 68:213 (1930); Lythgoe et al., J. Chem. Soc., 355 (1947); Lythgoe et al., J. Chem. Soc., 965 (1948); Davoll et al., J. Chem. Soc., 967 (1948); U.S. Pat. No. 6,509,320; H. Vorbrueggen and K. Krolikiewicz, Angew. Chem. Intl. Ed. 14, 421 (1975); T. F. Lai and R. E. Marsh, Acta Crystallogr., B28:1982 (1972). D. B. Davies and A. Rabczenko, J. Chem. Soc. Perkin Trans. 2:1703 (1975); Prog. Clin. Biol. Res., 230, 1-395 (1987); K G. Cunningham et al., J. Chem. Soc., 2299 (1951); U.S. Pat. No. 6,392,085; N. M. Kredich and A. J. Guarino, Biochim. Biophys. Acta, 41:363 (1960); U.S. Pat. No. 6,255,485; H. R. Bentley et al., J. Chem. Soc., 2301 (1951); U.S. Pat. No. 6,255,292; E. A. Kaczka et al., Biochem. Biophys. Res. Commun., 14, 456 (1964); R. Suhadolnik et al., J. Am. Chem. Soc., 86:948 (1964); A. R. Todd and T. L. Ulbricht, J. Chem. Soc., 1960:3275; W. W. Lee et al., J. Am. Chem. Soc. 83:1906 (1961); E. Walton et al., J. Am. Chem. Soc., 86:2952 (1964); Y. Ito et al., J. Am. Chem. Soc., 103:6739 (1981); RE37,045; H. T. Shigeura and G. E. Boxer, Biochem. Biophys. Res. Commun., 17:758 (1964); S. Penman et al., Proc. Nat. Acad. Sci. USA, 67, 1878 (1970); J. J. Fox. et al., Progr. Nucleic Acid Res. Mol. Biol., 5:258-262 (1966); A. J. Guarino, “Cordycepin” in Antibiotics I, D. Gottlieb, P. Shaw, Eds. (Springer-Verlag, New York, 1967) pp 468480; H. Klenow, Biochim. Biophys. Acta, 76:347 (1963); H. Shigeura and S. Sampson, Biochim. Biophys. Acta, 138:26 (1967), J. J. Novak and F. Sorm, Coll. Czech. Chem. Commun., 38:113 (1973); M. Blandin, J. Carbohyd. Nucl., Nucl., 3(5/6):341 (1976); Lecoq, Int. Z. Vitaminforsch., 27:291 (1957); Kossel, Ber., 18:79, 1928 (1885). Fischer, Ber., 30:2226 (1897); Traube, Ann., 331:64 (1904); Hoffer, Jubilee Vol. Emil Barell 428-434 (1946); Taylor et al., Ciba Foundation Symposium, Chem. and Biol. Purines, 1957, 20, C.A. 53, 6238b (1959); Bredereck et al., Angew. Chem., 71:524 (1959); Morita et al., Chem. & Ind. (London), 1117 (1968); Sekiya, Suzuki, Chem. Pharm. Bull., 20:209 (1972); N. J. Kos et al., J. Org. Chem., 44:3140 (1979); Philips et al., J. Pharmacol. Exp. Ther., 104:20 (1952); Hendrickson et al., Nucleic Acids Res. 2004;32(7):2241-50; Wang et al., Nucleosides Nucleotides Nucleic Acids. 2004;23(1-2):161-70; Gouder et al., J Neurosci. Jan. 21, 2004; 24(3):692-701; Singh et al., J Enzyme Inhib Med Chem. 2003 October; 18(5):395-402; Tkachenko et al., J Cell Sci. Jul. 1, 2004; 117(Pt 15):3189-99; Ekbom et al., Blood Press. 2004;13(3):137-141; Deruelle et al., Eur J Obstet Gynecol Reprod Biol. Jul. 15, 2004; 115(1):106-7; Li et al., J Appl Physiol. Jun. 25, 2004; Zhou et al., J Med Chem. Jan. 29, 2004; 47(3):566-75; Doláková et al., Nucleosides Nucleotides Nucleic Acids. 2003 December; 22(12):2145-60; Whitfield et al., Chem Commun (Camb). Nov. 21, 2003(22):2802-3; Okuyama et al., Biosci Biotechnol Biochem. 2003 May; 67(5):989-95; Ts'o, “Bases, Nucleosides and Nucleotides” in Basic Principles in Nucleic Acid Chemistry vol. 1, P.O.P. Ts'o, Ed. (Academic Press, New York, 1974) pp 453-584; U.S. Pat. No. 6,472,507; U.S. Pat. No. 6,579,857; U.S. Pat. No. 5,756,706; U.S. Pat. No. 5,744,596; U.S. Pat. No. 5,654,286; U.S. Pat. No. 5,541,164; U.S. Pat. No. 5,506,213; U.S. Pat. No. 5,310,732; US2004043955; EP0963997; U.S. Pat. No. 6,436,947; U.S. Pat. No. 4,283,394; and U.S. Pat. No. 4,138,562.

Other suitable agents include, e.g., Adenosine; 9-β-D-Ribofuranosyl-9H-purin-6-amine; 6-amino-9-β-D-ribofuranosyl-9H-purine; 9-β-D-ribofiranosidoadenine; adenine riboside; 3′-Deoxyadenosine; 9-cordyceposidoadenine; deoxyadenosine (2R,3S,5R)-5-(6-amino-9H-purin-9-yl)-tetrahydro-2-(hydroxymethyl)furan-3-ol; Cordycepin-5′-triphosphate; 3′-deoxy ATP; 3′-deoxyadenosine-5′-(tetrahydrogen triphosphate); 1H-Purin-6-amine; 6-aminopurine; 6-amino-1H-purine; 6-amino-3H-purine; 6-amino-9H-purine; 1,6-dihydro-6-iminopurine; 3,6-dihydro-6-iminopurine; β-D- and β-L-enantiomers of adenosine and adenine.

Other suitable agents also include, e.g., Adenocard (Fujisawa); Adenocor (Sanofi-Winthrop); Tubercidin; Amiloride; 2-Amino-6-chloropurine and Adenoscan (Fujisawa).

Additional agents useful in the present invention include adefovir (Hepsera®, Gilead, Foster City, Calif.), mycophenolic acid mofetil, non-hydrolyzable analogues of dATP, dGTP, dCTP and dTTP (e.g., dATP-γ-S), 2′2′difluorodeoxyguanosine (G version of Gemzar (gemcitabine; Lilly, Indianapolis, Ind.)), 2′2′difluorodeoxyadenosine (A version of Gemzar), and 2′2′difluorodeoxythymidine (T version of Gemzar). Also, ribose modifications traditionally used in oligonucleosides (RNA and DNA) to stabilize the oligos to nuclease degradation may also be useful in the present invention. These modifications are well known in the art (and many are described in patents assigned to ISIS, NeXstar Pharmaceuticals, Genta, Hybridon) and include, but are not limited to, 2′ methoxy and 2′fluoro derivatives of all natural nucleosides (e.g., 2′methoxy of dAdo, dAMP, dADP, dATP and 2′ fluoro of dAdo, dAMP, dADP, dATP; 2′methoxy of dTdo, dTMP, dTDP, dTTP and 2′ fluoro of dTdo, dTMP, dTDP, dTTP; 2′methoxy of dGdo, dGMP, dGDP, dGTP and 2′ fluoro of dGdo, dGMP, dGDP, dGTP; and 2′methoxy of dCdo, dCMP, dCDP, dCTP and 2′ fluoro of dCdo, dCMP, dCDP, dCTP).

Adenosine Deaminase (ADA)

ADA is an enzyme essential for the metabolism of purine nucleosides, one of which is 2′-deoxyadenosine (dAdo). The deamination of dAdo results in the formation of 2′-deoxyinosine, which is hydrolyzed to yield its purine base that is subsequently oxidized prior to conversion and removal of uric acid.(Wood, Drug Ther., 330:691 (1994)).

Deamination of dAdo by Adenosine Deaminase (ADA)

The deamination process is a hydrolysis reaction with ADA acting as a general base catalyst. As shown in Scheme 1, an unstable transition state is formed before deamination is completed and dAdo is metabolized to 2′-deoxyinosine (Klohs et al., Pharmacol. Rev., 44:459 (1992)). 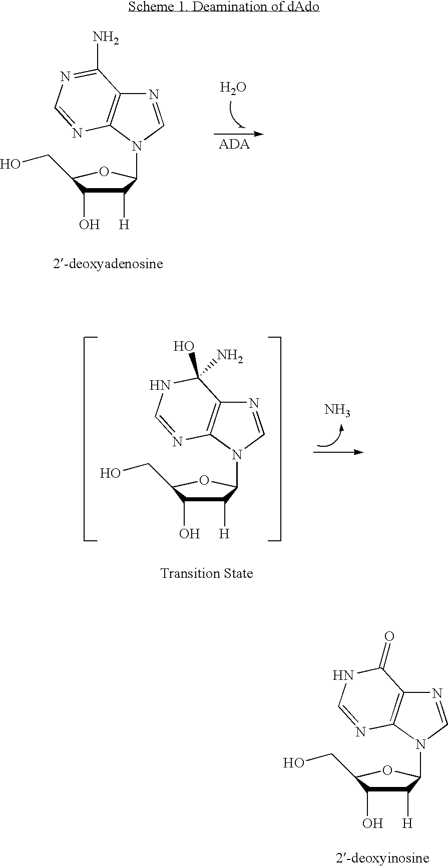
The Mechanism of ADA Inhibition by 2′-deoxycoformycin (dCF)

2′-deoxycoformycin (dCF), Scheme 2, (also known as Pentostatin or Nipent®) is produced by fermentation of Streptomyces antibioticus (Schramm, Biochemisty 24:641 (1985)) (SuperGen, Inc.) and Aspergillus nidulans (Niitsu et al., Blood, 92:3368 (1998)) (Chemo-Sero-Therapeutic-Research Institute). After purification, the C-8 R-isomer is collected and used as an ADA inhibitor with the full chemical name of (8R)-3-(2-Deoxy-β-D-erythro-pentofuranosyl)-3,4,7,8-tetrahydroimidazo[4,5-d]-[1,3]diazepin-8-ol. dCF has also been chemically synthesized. See, e.g., Chan, E. et al. J. Org. Chem. 47: 3457-3464 (1982).

dCF is considered a transition state analog inhibitor of ADA because it has similar chemical characteristics as the purported transition state analog of dAdo in Scheme 1 (Schramm, Biochemistry, 24:641 (1985)). Studies suggest that dCF has specific characteristics to cause a potent inhibitory binding effect to ADA. In 1985, it was revealed that the S-isomer of dCF at the C-8 position had less than 0.1% of the inhibitory activity as the R-isomer. In another study, the seven-membered ring was determined to be an important contributor to the binding of ADA because it holds the asymmetric center, at C-8, in the proper orientation relative to the transition state. (Montgomery et al., J. Med. Chem., 28:1751 (1985)) In addition, another dCF characteristic that makes it a potent inhibitor of ADA is that the sugar of the nucleoside contains the 2′-deoxyribose instead of the ribose, like its counterpart coformycin. This difference was studied and it was shown that dCF has about a 4-fold lower inhibition constant than coformycin revealing that the removal of an —OH group increases the strength of the inhibitor-enzyme complex. 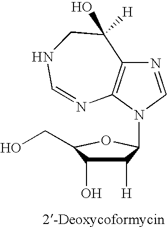
Scheme 2. 2′-Deoxycoformycin (Pentostatin) (Merck Index, 13^{th} Edition)
The Kinetics of 2′-deoxycoformycin (dCF) Binding to Adenosine Deaminase (ADA)

dCF is a competitive inhibitor of the ADA enzyme. It competes with purine nucleosides, primarily deoxyadenosine (dAdo), for the active site on the enzyme. dCF binding affinity is strong and the off-rate kinetics is extremely slow so the dCF-enzyme complex is kinetically similar to a covalent interaction. This interaction has been measured using steady-state kinetics. Kᵢ is the enzyme inhibitor constant determined from the ratio of k₂/k₁, where k₂ is the enzyme-inhibitor dissociation rate constant and k₁ is the enzyme-inhibitor association rate constant. (Agarwal et al., Biochem. Pharmacol., 26:359 (1977))

$$E + I {⇌}_{{k}_{2}}^{{k}_{1}} E I$$

Kᵢ measured from multiple studies was determined to be in the 10⁻¹² M range. (Kolesar et al., J. Oncol. Pharm. Practice, 2:211 (1996)) The k₂ and k₁ are 10⁻⁶ sec⁻¹ and 10⁶ M⁻¹ sec⁻¹, respectively. The corresponding half-life for the enzyme inhibitor complex was determined to be 68 hours. The data summarized above demonstrate that dCF is an extremely tightly bound inhibitor of ADA.

Inhibition of ADA leads to an elevation of deoxyadenosine (dAdo), dAMP, dADP and dATP triphosphate (dATP) in lymphocytes (Smyth et al., 1980; Ogawa et al., 2000; Johnston et al., 1992; Pettitt, 2003; Niitsu et al., 1996).

After dCF inhibits ADA, dAdo can no longer undergo deamination to 2′-deoxyinosine so inevitably dAdo concentrations increase. The dAdo is available to undergo phosphorylation by deoxycytidine kinase (dCK), which ultimately results in elevated dATP, as shown in Scheme 3. dATP can also be elevated if the AMP deaminase enzyme is present. If dAdo is processed through either enzyme the elevation of dATP will inhibit the ribonucleotide reductase, the enzyme that is responsible for the production of deoxyribonucleotides (dNTPs), which are the substrates for DNA synthesis.

It is also possible that dCK and the AMP deaminase are not active or present in the cell resulting in sustained elevated levels of dAdo. Elevated levels of dAdo can also occur if the ratio between 5′-nucleotidase to dCK is high triggering dAMP conversion back to its nucleoside. These elevated levels of dAdo could possibly inhibit SAH (S-adenosylhomocysteine) hydrolase, which leads indirectly to a reduction in DNA synthesis (see below). Alternatively, in dividing cells elevated levels of dATP can inhibit ribonucleotide reductase, thereby leading to a reduction of dNTPs used in DNA synthesis (see below).

Deoxycoformycin has detrimental effects on cycling (activated) as well as quiescent (resting) lymphocytes. In the dermis and epidermis of inflamed skin, multiple lymphocyte subsets exist in both functional states, so the potential for dCF to locally affect these cells is significant. 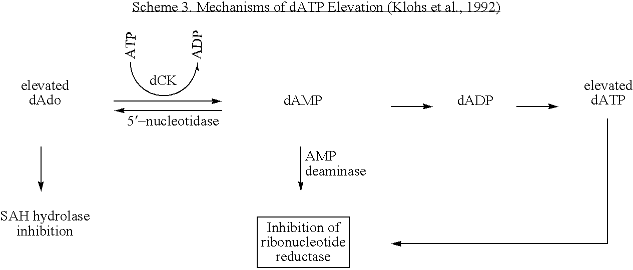
Elevated Levels of dAdo Lead to Elevated Levels of dATP, Which Inhibits Ribonucleotide Reductase, a Key Enzyme Needed for DNA Synthesis in Activated Lymphocytes

Ribonucleotide reductase (RNR) is an enzyme that reduces nucleotides to their respective deoxynucleotides (dNTP's). RNR levels fluctuate during the cell cycle with a marked elevation at the G1/S interface. The highest levels are present during the S-phase when there is a need for deoxyribonucleotide precursors for DNA synthesis. The RNR enzyme has two regulatory binding sites that are separate from the substrate-binding site. One site is the “overall activity site” and the other is the “substrate specificity site”. Only ATP and dATP can bind to the “overall activity site”. If ATP is bound, the enzyme is active; if dATP is bound, the enzyme is inactive. Thus, allosteric modulation of RNR by dATP/ATP controls the balance of dNTP's produced by the enzyme. Elevated levels of dATP resulting from the inhibition of ADA lead to the inhibition of RNR, thereby depleting the intracellular pool of dNTP's and stalling DNA synthesis.

Another explanation is that elevated levels of dAdo may produce elevated levels of AMP, which is subsequently deaminated by AMP deaminase to deoxyinosine-5′-monophosphate (dIMP), as shown in Scheme 5. dIMP is metabolized by two pathways. If dIMP is metabolized by hypoxanthine guanine phosphoribosyl transferase, then ADA inhibition is abrogated resulting in the production of 2′-deoxyinosine, which is the deaminated nucleoside of dAdo. If dIMP is metabolized by adenylosuccinate, then dATP accumulation occurs, resulting in the inhibition of RNR and the stalling of DNA synthesis.

Once ribonucleotide reductase is inhibited, DNA synthesis will be impaired due to the low levels of dNTP's. Thus, cells that must replicate DNA and divide in order to be functional (T-cells, for instance) are affected.

Elevated Levels of dAdo Lead to Inhibition of SAH Hydrolase in Lymphocytes

Methyltransferases catalyze the transfer of single carbon donor groups from cofactors to intermediary metabolites, a process termed post-translational methylation. S-adenosylmethionine (SAM) is an important methyl donor substrate used by these enzymes. Two important groups of intermediary metabolites that accept methyl groups are purine and pyrimidine nucleosides. (Klohs and Kraker, Pharmacol. Rev., 44:459 (1992)) A physiological competitive inhibitor of methyltransferases is S-adenosylhomocysteine (SAH). Methyltransferase inhibition by SAH is usually less prominent than the binding of SAM, the major methyl donor substrate. Thus, the SAM to SAH ratio is normally high, and under normal steady state conditions, there is a net methylation of acceptor molecules. After SAM is used in the methylation process, SAH is formed and subsequently hydrolyzed by SAH hydrolase to maintain a balanced SAM to SAH ratio. (Ho, Blood, 72:1884 (1988))

As shown in Scheme 3, if the ratio between 5′-nucleotidase to dCK is high or if neither enzyme is active then the levels of dAdo will begin to elevate after ADA is inhibited. In the presence of elevated levels of dAdo, SAH hydrolase is inhibited causing an elevation of SAH levels in the cell. Increased SAH competitively inhibits SAM in a variety of methylation reactions required for purine and pyrimidine biosynthesis, as well as other metabolic activities such as methylation of cytosine residues in chromosomal DNA. This leads to a decrease in DNA synthesis, inhibition in the power to repair DNA damage, and ultimately compromises cell viability.

Scheme 4 illustrates the normal mechanism by which SAM is metabolized and the mechanism of reaction when dAdo levels become elevated and SAH hydrolase is inhibited.

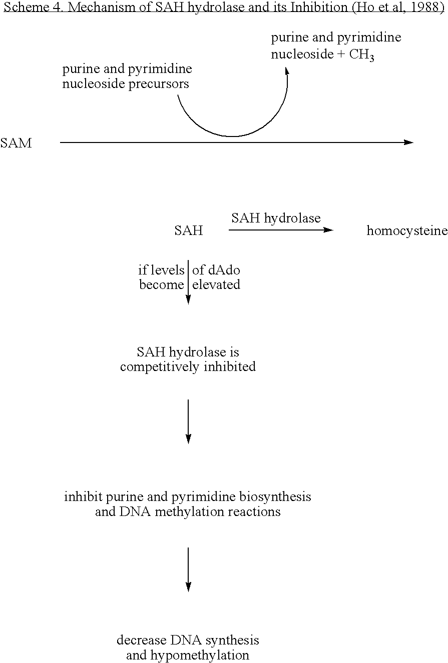

In resting lymphocytes dCF induced cytotoxicity was accompanied with inhibited SAH hydrolase enzymes demonstrating that in non-dividing cells where ribonucleotide reductase is low, the mechanism of cell death is at least partially attributed to the inhibition of the SAH hydrolase. (Ho, Blood, 72:1884 (1988)).

Elevated Levels of dATP Promote Activation of Poly(ADP-ribose) Polymerase (PARP), an Important Nuclear Enzyme Linked to DNA Repair and Apoptosis in Lymphocytes

PARP is a nuclear enzyme that is associated with the repair of DNA strand breaks. PARP has an N-terminal DNA binding site that binds to DNA strand breaks. After a strand break is recognized and bound by the N-terminal domain, the C-terminal catalytic domain of PARP is activated and catalyzes the conversion of NAD⁺ (nicotinamide adenine dinucleotide) to ADP-ribose. The automodification domain of PARP binds to the ADP-ribose formed. PARP synthesizes poly(ADP-ribose) modifications used in the repair of the DNA strand breaks. In addition, PARP catalyzes poly(ADP-ribosylation) of other nuclear proteins, such as histones, and this modification regulates various functions such as transcription (Bernges et al.: Functional Overexpression of Human Poly(ADP-ribose) Polymerase in Transfected Rat Tumor Cells, Carcinogenesis, 18(4):663-8 (1997)).

In resting lymphocytes, it is known that, as part of the immune response system, DNA strands are continually breaking and rejoining. It has been shown that dATP inhibits the repair of these spontaneously occurring DNA breaks. Elevation of dATP will result in the accumulation of DNA breaks causing hyper-activation of PARP (Bernges et al.: Functional Overexpression of Human Poly(ADP-ribose) Polymerase in Transfected Rat Tumor Cells; Meli et al., Toxicology Lett., 139:153 (2003)).

Constitutive PARP activation results in the depletion of NAD⁺ by the formation of ADP-riboses. NAD⁺ is regenerated from ATP, so consequently ATP is depleted and the cell has a net depletion of energy. (Meli et al., Toxicology Lett., 139:153 (2003)). DNA synthesis and other critical processes become impaired.

Elevated Levels of dATP Promote Apoptosis in Lymphocytes via Multiple Mechanisms

Elevation of dATP can activate PARP as described above, and this may contribute to the induction of apoptosis. However, dATP has the capacity to promote apoptosis by additional mechanisms, including the activation of caspase enzymes and ultimately the fragmentation of genomic DNA, which is a hallmark of apoptosis. (Widlak, Acta Biochimica Polonica, 47:1037 (2000))

At the point that DNA single-strand breaks begin to accumulate after the elevation of dATP induced by dCF, a cysteine protease, known as Caspase-8, is activated, thereby increasing cytosolic Ca⁺⁺ ion concentrations, which in turn induce a permeability transition of the mitochondrial membrane. (Yang and Cortopassi, Biochem. Biophys. Res. Comm., 250:454 (1998)) Caspase-8 mediated cleavage can release cytochrome c from the mitochondria through the ion activated channels. It has also been observed that elevated levels of dATP can cause a release of cytochrome c from the mitochondria without the activation by Caspase-8. (Widlak, Acta Biochimica Polonica, 47:1037 (2000))

Extramitochondrial (cytoplasmic) cytochrome c is pro-apoptotic, because it can activate the full caspase cascade. Cytosolic cytochrome c binds to Apaf-1 (Apoptosis protease-activating factor-1), which in turn induces auto-activation of another cysteine protease known as caspase-9, which in turn activates caspase-3. Alternatively, caspase-8 and PARP have been shown to directly activate caspase-3. Caspase-3 is termed the “major executioner caspase” because it is the most “downstream” cysteine protease, and has numerous cytoplasmic and nuclear substrates. (Widlak, Acta Biochimica Polonica, 47:1037 (2000); Leoni et al., Proc. Natl. Acad. Sci., 95:9567 (1998))

One of the most prominent substrates for caspase 3 is DNA Fragmentation Factor40 (DFF40), a Mg⁺⁺ dependent endonuclease that is responsible for genomic DNA fragmentation that is associated with apoptosis (Widlak, Acta Biochimica Polonica, 47:1037 (2000)). DFF40 creates double strand breaks that trigger chromatin condensation, characteristic of apoptotic cells. DFF40 is produced following a cascade of apoptotic signals and reactions. DFF40 is an inactive cytosolic heterodimeric protein that consists of two subunits, one at 45 kd and another at 40 kD. DFF is activated by caspase-3 mediated cleavage into the two subunits, DFF45 and DFF40 kD. Once DFF is activated, DFF45 and DFF40 work together to fragment the DNA. DFF45 is considered the chaperon because it contains the regulatory unit that leads DFF40 to the DNA. DFF40 contains the catalytic function that is involved with the double-strand cleavage of the DNA.

There is an emerging consensus that dCF induces cell death through a complex series of biochemical reactions that ultimately promote apoptosis. Apoptosis is characterized by morphologic changes such as cell shrinkage, mitochondrial cytochrome c release, chromatin condensation, and fragmentation of cell DNA. Apoptosis is a programmed mechanism of cell death. As summarized above, recent in vitro experiments have identified apoptosis as the primary mechanism of lymphocyte and monocyte cell death following treatment with dCF or dCF plus dAdo. 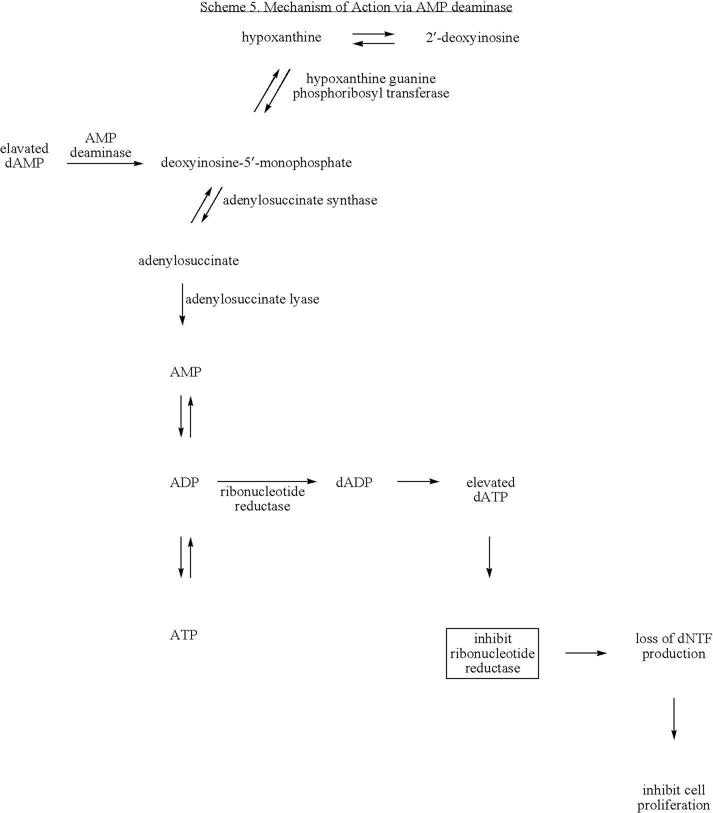
Deoxycoformycin is Toxic to Multiple Subsets of Lymphocytes

Not only does dCF target T-cell lymphocyte subsets (CD4⁺/CD8⁺), which play a significant role in T_{H}1/T_{H}2 type inflammation, but also there are multiple additional potential immune cell targets of dCF. dCF inhibits the growth of B-cell lines, which may directly interfere with the production of antibodies. Niitsu et al., Blood, 92:3368 (1998). When this occurs, the T_{H} cells 10 that detect foreign peptides will not be able to stimulate B-cells to produce the antibodies. Below is a description of some of the mechanisms by which lymphocytes are affected by dCF. In lymphocytes, these mechanisms may be different depending upon the presence of ADA and other enzymes in the cell, the developmental lineage of the cell and its stage in the development process.

Deoxycoformycin, Agents Resistant to Deamination (e.g., Cladribine) and dAd (and Analogs Thereof) are Toxic to Quiescent (Resting) Lymphocytes

Deoxycoformycin (dCF) is known to induce apoptosis in T-cells and monocytoid cells through the inhibition of adenosine deaminase (ADA). As described herein dAdo also induces apoptosis in T-cells. The exposure of cells to dCF and deoxyadenosine (dAd) leads to greater levels of apoptosis than dCF alone; and the addition of dAd reduces the effective dose of dCF required to induce apoptosis (Niitsu et al. 1999; Niitsu et al. 2000; Bagnara, et al. 1992). In other words, dAd potentiates the pro-apoptotic effect of dCF on T-cells and monocytoid cells.

In resting lymphocytes, the inhibition of SAH hydrolase was thought to be a possible mechanism in which dCF caused cells to die. However, because the ratio of dCK to 5′-nucleotidase is high in lymphocytes it is unlikely that SAH hydrolase is the principle mechanism of action but rather the elevated levels of dATP's.

In in vitro experiments, apoptosis has been associated with elevated levels of dATP, so dATP seems to be strongly linked to the induction of apoptosis. In addition, elevated levels of dATP have also been documented in leukemic patients treated with dCF.

Elevated levels of dATP inhibit RNR, which results in the accumulation of single stranded DNA breaks that cannot be repaired due to a depletion of dNTPs. The single stranded breaks in turn activate PARP, which leads to the depletion of ATP and subsequently cell death. Also, elevated levels of dATP may be sufficient to release proapoptotic cytochrome c from the mitochondria without PARP. Thus, dCF, dAd, and a combination of dCF and dAd, can induce apoptosis in lymphocytes that are not cycling.

Additionally, agents resistant to deamination, such as cladribine, can also result in efficient induction of apoptosis of non-cycling lymphocytes (e.g., as a monotherapy). For example, Cladribine is phosphorylated by dCK and dGK to the metabolically active nucleoside triphosphate, CdATP. After phosphorylation, CdATP is incorporated into DNA synthesis as an internal part of the DNA sequence. In addition, CdATP has weak resistance to 3′ to 5′ excision, so once CdATP is incorporated into the DNA the effect can be negated more efficiently. CdATP also inhibits RNR. Once RNR is inhibited, dATP and dCTP levels will be depleted. This allows CdATP to be incorporated into DNA synthesis more efficiently because there is less competition with dATP. The depletion of dCTP will cause an upregulation of dCK making CdA more susceptible to phosphorylation. Because of the internal incorporation of CdATP into DNA synthesis, the effect on quiescent cells may be greater than on dividing cells. In resting cells, elevated levels of CdATP will cause an accumulation of single strand DNA breaks that is presumed to activate the enzyme poly (ADP-ribose) polymerase (PARP). Upregulation of PARP leads to cellular loss of nicotinamide adenine dinucleotide (NAD), which is a cofactor in energy (ATP) production, resulting in ATP depletion and subsequent loss of cell function.

Deoxycoformycin, Agents Resistant to Deamination (e.g., Cladribine) and dAd are Toxic to Cycling (Activated) Lymphocytes

dCF, agents resistant to deamination, such as cladribine, and dAd (and analogs thereof) can induce apoptosis in cycling (activated) lymphocytes. The mechanism by which apoptosis is triggered seems to differ in certain aspects from the mechanisms operating in quiescent (resting) lymphocytes. As explained above, the depletion of dNTP's due to the inhibition of RNR by dATP causes a cessation of DNA synthesis during S-phase. In addition, the cells ability to repair DNA is hindered and this may lead to constitutive activation of PARP as in resting lymphocytes.

Deoxycoformycin, Agents Resistant to Deamination (e.g., Cladribine) and dAd are Toxic to Cells in the Monocyte-macrophage Lineage

The growth of monocytes and monocyte cell lines is inhibited by dCF in vitro. dCF also inhibits the growth of peripheral monocytes. In addition, an in vitro study showed that monocytoid leukemia cells are more sensitive to dCF than normal monocytes. (Niitsu et al., Blood 92:3368 (1998))

In vivo, dCF has been used in some studies to treat monocytic derived disorders such as malignant histiocytosis, a hematolymphoid neoplasm characterized by the accumulation of malignant histiocytes (macrophages) in the reticuloendothelial system. (Weitzman et al., Med. Pediatr. Oncol., 33:476 (1999)). This confirms that dCF can affect immune cells other than lymphocytes.

Thus, dCF can induce apoptosis in multiple immune cell subsets. Therefore, when dCF is used to treat autoimmune diseases, it has polyclonal targeting properties, in contrast to its effects in monoclonal proliferations, which characterize leukemia and lymphoma.

Additionally, agents resistant to deamination (e.g., cladribine) are toxic to cells in the monocyte-macrophage lineage. The mechanism is analogous to that of dCF.

The Aqueous Instability of dCF

dCF has been evaluated for stability in aqueous solutions at pH ranging from 1 to 12.7 at 25° C. The degradation products were isolated and identified. The proposed mechanism for degradation due to acid-catalyzed hydrolysis and base-catalyzed hydrolysis are shown below in Schemes 6-7. (Al-Razzak et al., Pharmaceut. Res., 7:452 (1990)).

In Scheme 6, the protonation of the two nitrogen atoms is the initial reaction that leads to the cleaving of 2′-deoxyribose from the purine base. The kinetics of the reaction increases as the pH is reduced, because the H⁺ ion concentration increases. After protonation, the nitrogen bonds are stabilized by the donation of electrons from the oxygen on 2′-deoxyribose, which leads to the separation of the base and sugar. At a pH of 1, t₅₀ is 5.6 minutes and at pH 5.0 t₅₀ is about 3.5 days.

As the pH increases, water and a proton interacts with the amidine to cause the 7-membered nitrogenous base (heterocyclic ring) of the purine to open (Scheme 7). The reaction is a relatively slow, since the combination of water in conjunction with a proton in a basic system is limited because the concentration of H⁺ is relatively low in the solution. The most stable pH appears to be 7.5 where t₅₀ is approximately 70 days. At pH 12.6 the t₅₀ is approximately 45 days. Thus, the higher the pH the more the basic conditions catalyze the hydrolysis of dCF.

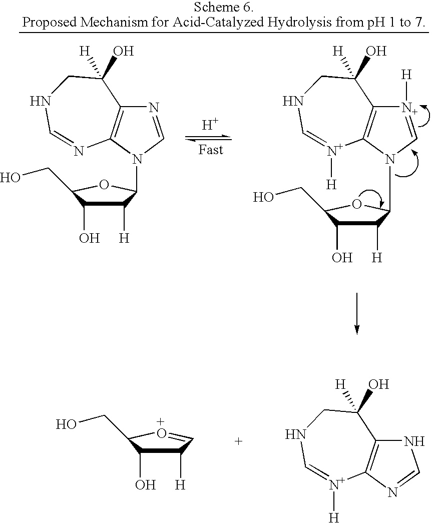

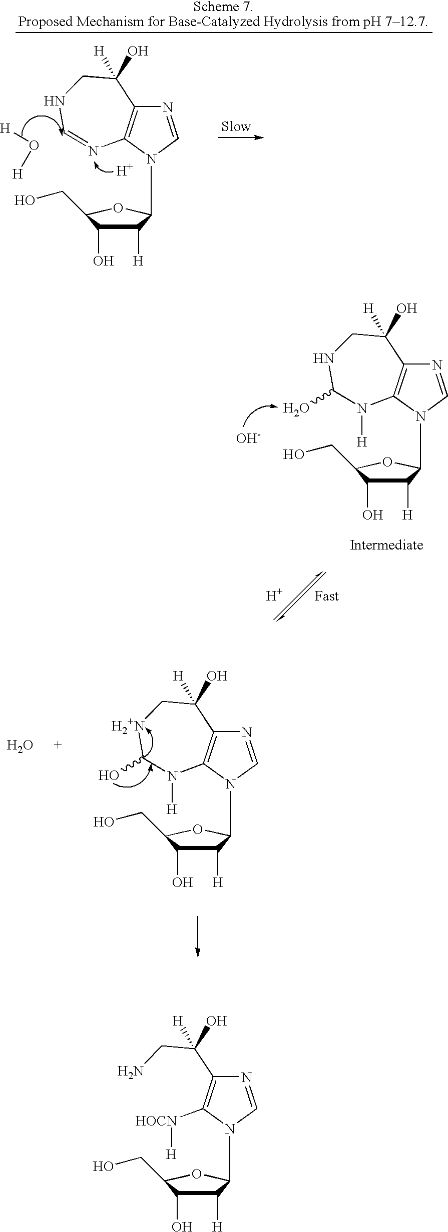

dCF degradation involves a hydrolysis reaction that takes place within a pH range of 5 to 12. The hydrolysis reaction can be quenched or diminished by minimizing the amount of water present in the composition. As such, for a one-part system, the composition will typically include dCF combined with a base topical delivery system, such as a solution, gel, cream, or ointment. The concentration of water varies with each formulation where a solution base may have about 25-80 w/w % water; a gel base may have about 25-95 w/w % water; a cream base may have about 50-80 w/w % water; and an ointment base may have between about 1-10 w/w % water. The rate of hydrolysis is lowest in the pH range of 7 to 9, so all formulations are buffered respectively.

Given the stability of dCF when lyophilized, a two-part formulation would include dCF lyophilized in, for example, Syringe A and a topical vehicle in, for example, Syringe B. Prior to administration, the dCF and delivery components could be mixed thoroughly by repetitively transferring the components between the syringes. In this way, the product is reconstituted. A two-part (mix and use) formulation minimizes the probability of dCF hydrolysis because the drug is exposed to water only very briefly prior to application. A preferred two-part formulation uses a cream base; however, a gel or ointment base may also be implemented if necessary. These formulations are also sufficient for other agents, such as, dAdo or agents which are resistant to deamination (e.g., cladribine) or which inhibit DNA synthesis (directly or indirectly).

Topical Therapeutic Formulation

The topical preparation (delivery system or pharmaceutical formulation) will include at least one adenosine deaminase inhibitor, antiviral agent, antimetabolite agent, natural metabolite, nucleoside, nucleotide, purine base, pyrimidine base or an analog thereof, including, but not limited to, dAdo and cladribine, to be delivered to inflamed skin. The adenosine deaminase inhibitor, antiviral agent, antimetabolite agent, natural metabolite, nucleoside, nucleotide, purine base, pyrimidine base or an analog thereof may be used alone or in combination in the present compositions. The adenosine deaminase inhibitor, antiviral agent, antimetabolite agent, natural metabolite, nucleoside, nucleotide, purine base, pyrimidine base or an analog thereof is capable of providing local or systemic biological or physiological activity in an animal, including a human.

The adenosine deaminase inhibitor, antiviral agent, antimetabolite agent, natural metabolite, nucleoside, nucleotide, purine base, pyrimidine base or an analog thereof may be soluble in the vehicle to provide a homogeneous solution in the delivery system. Alternatively, the adenosine deaminase inhibitor, antiviral agent, antimetabolite agent, natural metabolite, nucleoside, nucleotide, purine base, pyrimidine base or an analog thereof may be insoluble in the vehicle to form a suspension or dispersion with the vehicle. Further, the adenosine deaminase inhibitor, antiviral agent, antimetabolite agent, natural metabolite, nucleoside, nucleotide, purine base, pyrimidine base or an analog thereof may be soluble in the vehicle and it may be added to the composition in an amount to saturate the vehicle and have additional undissolved adenosine deaminase inhibitor, antiviral agent, antimetabolite agent, natural metabolite, nucleoside, nucleotide, purine base, pyrimidine base or an analog thereof in a suspension or dispersion.

The composition may be prepared by first combining a adenosine deaminase inhibitor, antiviral agent, antimetabolite agent, natural metabolite, nucleoside, nucleotide, purine base, pyrimidine base, analog thereof or combination thereof with or without stabilizing additives to form a mixture. This mixture may be physically and chemically stable for long-term storage. The mixture is combined with the delivery vehicle prior to administration to the skin. It is highly preferred that the adenosine deaminase inhibitor, antiviral agent, antimetabolite agent, natural metabolite, nucleoside, nucleotide, purine base, pyrimidine base or an analog thereof /stabilizing additive mixture be combined with the delivery vehicle almost immediately prior to administration.

The composition contains the adenosine deaminase inhibitor, antiviral agent, antimetabolite agent, natural metabolite, nucleoside, nucleotide, purine base, pyrimidine base or an analog thereof in an amount effective to provide a desired biological, physiological, pharmacological, and/or therapeutic effect, optionally according to a desired release profile, and/or time duration of release. It is further preferred that the adenosine deaminase inhibitor, antiviral agent, antimetabolite agent, natural metabolite, nucleoside, nucleotide, purine base, pyrimidine base or an analog thereof is included in the vehicle in an amount effective to provide an acceptable solution or dispersion viscosity.

For the objectives of the present invention, the adenosine deaminase inhibitor may be selected from the group of cladribine, deoxycoformycin (pentostatin, Nipent®), coformycin, diethyl pyrocarbonate, erythro-9-(2-hydroxy-3-nonyl) adenine, erythro-9-[3-(2-hydroxynonyl)]adenosine, erythro-9-(2-hydroxy-3-nonyl)-adenosine (EHNA), 6-(R)-hydroxyl-1,6-dihydropurine ribonucleoside (HDPR), imidazole-4-carboxamide derivatives, erythro-6-amino-9(2-hydroxy-3-nonyl)-purine hydrochloride, erythro-9-(2-hydroxy-3-nonyl)-3-deazaadenine, 1-deazaadenosine, Adenosine, 2-cyano-2′,3′-dideoxy-, Adenosine, 2′,3′-dideoxy-2-ethyl-, Adenosine, 2′,3′-dideoxy-2-(methylthio)-, Adenosine, 2′,3′-dideoxy-2-(trifluoromethyl)-, 2′,3′-Dideoxy-2-iodoadenosine, (+/−)-9H-Purine-9-ethanol, 6-amino-β-hexyl-α-methyl-, and analogs and combinations thereof. In another specific embodiment of the present invention, the adenosine deaminase inhibitor can be (R)-3-(2-Deoxy-β-D-erythro-pentofuranosyl)-3,6,7,8-tetrahydroimidazo[4,5-d][1,3]diazepin-8-ol.

Additionally, other biologically active agents could be included with the current invention or co-administered with the host product that synergizes with the adenosine deaminase inhibitor to produce apoptosis in lymphocytes, antigen presenting cells or their precursors. Examples of suitable co-administered (e.g., simultaneous or sequentially administered) agents include, e.g., adenosine nucleotides, adenosine nucleosides, adenosine bases and analogs thereof, including dAdo.

In a specific embodiment of the invention, the adenosine deaminase inhibitor is deoxycoformycin. Deoxycoformycin is an antimetabolite isolated from Streptomyces antiobiotiucus or Aspergillus nidulans, and is also known as Pentostatin. Chemically, the drug is (R)-3-(2-Deoxy-β-D-erythro-pentofuranosyl)-3,6,7,8-tetrahydroimidazo[4,5-d][1,3]diazepin-8-ol. The drug is commercially available for the treatment of hairy cell leukemia In such a specific embodiment, the deoxycoformycin may or may not be administered with additional medicates for synergistic efficacy augmentation from the class of compounds described above.

In a specific embodiment of the invention, the antimetabolite is cladribine. A compound which is mostly resistant to deamination and interferes with DNA synthesis. In another specific embodiment of the invention, the nucleoside analog is dAdo.

In a specific embodiment, deoxycoformycin, cladribine or dAdo is filled into a mixing container so that they remain in a solid, thus more stable state. The mixing container will allow for connection with a second container filled with a vehicle.

pH Range

Many compounds for use in the present invention are stable in specific pH ranges. For example, dCF has been evaluated for stability in aqueous solutions at pH ranging from 1 to 12.7 at 25° C. The degradation products were isolated and identified. The proposed mechanism for degradation due to acid-catalyzed hydrolysis and base-catalyzed hydrolysis are described, e.g., Al-Razzak et al, Pharmaceut. Res., 7:452 (1990).

It is the relative instability of dCF, and other compounds for use in the present invention, in aqueous environments that guides formulation development of this molecule. The most stabilizing formulations minimize water content in the pH range of 5 to 12 to minimize hydrolysis. The Examples herein provide suitable exemplary formulations that minimize water content.

Specifically, the pH of the composition can be about 5 to about 12. More specifically, the pH of the composition can be about 5.5 to about 11.5. More specifically, the pH of the composition can be about 6.0 to about 11.0. More specifically, the pH of the composition can be about 6.5 to about 10.5. More specifically, the pH of the composition can be about 5.5 to about 9.5. More specifically, the pH of the composition can be about 5.5 to about 8.5. More specifically, the pH of the composition can be about 7.0 to about 9.0.

The above pH ranges can be achieved with a suitable acid and/or base. It is appreciated that specific acids and/or bases, effective to achieve the above pH ranges, are known to those of skill in the art. See, e.g., Sigma Catalogue, 2004-2005 (St. Louis, Mo.); Aldrich Catalogue, 2004 (Milwaukee, Wis.); and Chemistry, Chang, 3^{rd} Ed., 1988, Random House (NY, N.Y.).

The above pH ranges can be maintained with a suitable buffer. It is appreciated that specific buffers, effective to maintain the above pH ranges, are known to those of skill in the art. See, e.g., Sigma Catalogue, 2004-2005 (St. Louis, Mo.); Aldrich Catalogue, 2004 (Milwaukee, Wis.); and Chemistry, Chang, 3^{rd} Ed., 1988, Random House (NY, N.Y.).

Water Content

dCF, and other compounds for use in the present invention, is unstable in aqueous solutions. Specifically, dCF forms degradation products by hydrolysis reactions. In a topical formulation, a two-part formulation can be employed, as described herein, so that the product will be stable. Alternatively, if the water is reduced below a minimal threshold, the hydrolysis reaction may be quenched, making a one-part product feasible.

Specifically, when water is present, it can be present up to about 95% (w/w) of the composition, up to about 90% (w/w) of the composition, up to about 85% (w/w) of the composition, or up to about 80% (w/w) of the composition. Typically, when the topical formulation is a solution, water can be present in about 25% (w/w) to about 80% (w/w) water. Typically, when the topical formulation is a gel, water can be present in about 25% (w/w) to about 95% (w/w) water. Typically, when the topical formulation is a cream, water can be present in about 50% (w/w) to about 80% (w/w) water. Typically, when the topical formulation is an ointment, water can be present in about 1% (w/w) to about 20% (w/w) water.

Vehicle

The vehicle capable of delivering the adenosine deaminase inhibitor, antiviral agent, antimetabolite agent, natural metabolite, nucleoside, nucleotide, purine base, pyrimidine base, analog thereof or combination thereof upon contact with the skin includes ingredients that may be, but are not limited to antimicrobial preservatives, emulsifying and/or solubilizing agents, humectants, ointment bases, solvents, stiffening agents, viscosity-inducing agents, and wetting agents. Other ingredients may be present that pertain to performance and elegance. Multiple formulations may be devised to provide complementary delivery platforms such as ointments, creams, gels, and lotions. Pharmaceutical compositions of the invention include cosmetic compositions. For example, one embodiment of the invention provides for cosmetic compositions including a adenosine deaminase inhibitor, antiviral agent, antimetabolite agent, natural metabolite, nucleoside, nucleotide, purine base, pyrimidine base, analog thereof or combination thereof. Suitable solvents for the present invention include mineral oil, propylene glycol and its derivatives, wax, natural and synthetic oils, and water. In one embodiment of the invention, the solvent is water to make a cream application where water includes the continuous phase. The cream vehicle is filled into a second container for mixing with the adenosine deaminase inhibitor, antiviral agent, antimetabolite agent, natural metabolite, nucleoside, nucleotide, purine base, pyrimidine base or an analog thereof.

Upon requirement for the adenosine deaminase inhibitor, antiviral agent, antimetabolite agent, natural metabolite, nucleoside, nucleotide, purine base, pyrimidine base, analog thereof or combination thereof, the container with the adenosine deaminase inhibitor, antiviral agent, antimetabolite agent, natural metabolite, nucleoside, nucleotide, purine base, pyrimidine base, an analog thereof, or combination thereof and the container with the vehicle would be connected in such a manner to allow mixing between the two containers. The product is blended to uniformity with agitation vigorous enough to ensure uniform dispersion of the adenosine deaminase inhibitor within the vehicle.

Dosage and Formulation

One of skill in the art can effectively determine the concentration of topical deaminase inhibitor, such dCF, antiviral agent, antimetabolite agent (e.g., cladribine), natural metabolite (e.g., deoxyadenosine), nucleoside, nucleotide, purine base, pyrimidine base or an analog thereof (e.g., dAdo and cladribine) needed to induce apoptosis, and/or to inhibit proliferation of, monocytes and/or lymphocytes in vivo, in situ or in vitro. Experiments conducted in vitro have generally included dAdo to potentiate the effect of dCF. Using the combination of drugs, effective cell killing by apoptosis usually takes 2-4 days. The degree of penetration of each drug into the dermis is unknown, so formulations are being developed using a combination of empirical and inductive experiments. The concentration range for a deaminase inhibitor, such as dCF, antiviral agent, antimetabolite agent, natural metabolite, nucleoside, nucleotide, purine base, pyrimidine base or an analog thereof (e.g., dAdo and cladribine) is guided by safety data from clinical studies. Based upon in vitro and in vivo data, it is anticipated that a deaminase inhibitor, such as dCF, antiviral agent, antimetabolite agent, natural metabolite, nucleoside, nucleotide, purine base, pyrimidine base or an analog thereof (e.g., dAdo and cladribine) monotherapy may be effective. However, the effect may take many days to gain momentum. On the other hand, if dCF (or another deaminase inhibitor) and dAdo (or another nucleoside analog) are combined, either concurrently or sequentially, much lower levels of dCF (or another deaminase inhibitor) may have activity, and the treatment duration may be reduced due to a more rapid rate of apoptosis of lymphocytes and monocytes within the epidermis and/or dermis.

The oncology dose for dCF is 4 mg/m² every 14 days. (Grever et al., J. Clin. Oncol., 3:1196 (1985)) Based upon the assumption that the average body surface area is 1.8 m², a safe dose of dCF is 7.2 mg every 14 days or 0.51 mg per day. For oncology patients, at this dosage, the adverse events were mild to moderate and diminished with treatment, therefore 4 mg/m² is referred to as the safe dose that can be administered systemically. At this dose range, patients with hairy cell leukemia (HCL) achieve responses in the first few weeks, and complete remissions, on average, after 6 months of treatment. (Kraut et al., Blood, 68:1119 (1986)).

Based upon topical formulations that are similar to the formulations of dCF, it is estimated that the systemic absorption of dCF will be no more than 7%. Calculations of safety margins have been performed, based upon this assumption and the known toxicity profile of dCF when administered IV for oncology indications, and for rheumatoid arthritis. Systemic absorption is a concern, because of the potential for serious adverse events, such as lymphopenia and renal toxicity. At oncology doses, these adverse reactions are usually minimized, but the therapeutic index is narrow. Another factor that plays a role in the safety of the formulation is the amount of product administered each day. Approximately 4 grams, 12 grams, and 40 grams of a topical formulation are estimated to cover 10%, 30%, and 100% of the body surface area. Provided herein are some examples of safety margin calculations using dCF monotherapy, based upon the assumption of 7% estimated fractional absorption, and various dose intensities.

The topical compositions described herein can include an adenosine deaminase inhibitor (e.g., dCF) in any suitable, effective and appropriate amount. Typically, the adenosine deaminase inhibitor (e.g., dCF) can be present in an amount from about 0.00005 wt. % to about 0.10 wt. % (e.g., about 0.0005 wt. %, about 0.005 wt. %, about 0.05 wt. %, or about 0.10 wt. %). In another specific embodiment of the present invention, the topical composition can include an adenosine deaminase inhibitor (e.g., dCF) in an amount from about 0.5 ug/mL to about 1,000 ug/mL (e.g., about 5 ug/mL, about 50 ug/mL, or about 500 ug/mL). In another specific embodiment of the present invention, the topical composition can include an adenosine deaminase inhibitor (e.g., dCF) in an amount from about 2 uM to about 3728 uM (e.g., about 19 uM, about 186 uM, about 1864 uM or about 3728 uM).

Using the highest concentration of 0.10% (1.0 mg/g), 10% body surface area coverage (4 grams) and assuming 7% systemic absorption, patients would be systemically exposed to 0.28 mg of dCF per day. This corresponds to a safety margin of 1.8. For the subsequent lower concentrations, the safety factor would be 18, 180, and 1,800.

The topical compositions described herein can include a nucleotide, nucleoside, purine base, pyrimidine base, or analog thereof (e.g., deoxyadenosine (dAdo) or adenosine arabinoside (AraA)) in any suitable, effective and appropriate amount. Typically, an adenosine nucleoside, adenosine nucleotide, adenosine base, or an analog thereof (e.g., cladribine or dAdo) can be present in an amount from about 0.00005 wt. % to about 5.0 wt. % (e.g., about 0.00005 wt. %, about 0.01 wt. %, about 0.05 wt. %, or about 0.10 wt. %, about 0.50 wt. %, about 1.0 wt. %, about 3.0 wt. %, or about 5.0 wt. %). In another specific embodiment of the present invention, the topical composition can include an adenosine nucleoside, adenosine nucleotide, adenosine base or analog thereof (e.g., cladribine or dAdo) in an amount from about 5.0 ug/mL to about 50,000 ug/mL (e.g., about 50 ug/mL, about 100 ug/mL, about 500 ug/mL, about 1000 ug/mL, about 5000 ug/mL, about 10,000 ug/mL, about 30,000 ug/mL, or about 50,000 ug/mL). In another specific embodiment of the present invention, the topical composition can include an adenosine nucleotide, adenosine nucleoside, adenosine base or an analog thereof (e.g., cladribine or deoxyadenosine (dAdo)) in an amount from about 0.02 mM to about 199 mM (e.g., about 0.2 mM, about 0.4 mM, about 2 mM, about 4 mM, about 20 mM, about 40 mM, about 119 mM, or about 199 mM).

The concentrations of dCF, cladribine and dAd provided herein are estimates, based upon an assumed penetration of drug, and the concentration ranges of dCF that inhibit the growth of lymphocytes and monocytes in vitro, when dCF is combined with dAdo. (Niitsu et al., Blood, 92:3368 (1998); and Niitsu et al., Blood, 96:1512).

Assuming that intracellular levels of dAdo are sufficient to generate elevated levels of dATP, the examples of single agent (monotherapy with, for example, dCF) should be adequate to treat patients with autoimmune diseases. The most dose intensive regimen may use up to 40 grams of a 0.01% dCF topical formulation delivered each day. This treatment regimen is 1.8 times safer than the oncology dose. For most patients with mild-to-moderate topical autoimmune conditions, the dose intensity is less than 12 grams per day. At 12 grams per day this treatment is approximately 6 times safer than the oncology dose of dCF. To improve the therapeutic index, the treatment schedule could be prolonged while using reduced concentrations of dCF thereby increasing the safety margins in the range of 100 to 36000. Based upon the calculations listed above, and the ability to stabilize the molecule by minimizing the hydrolysis, it is concluded that dCF may be used as a safe topical treatment for mild, moderate and severe autoimmune skin diseases.

The adenosine deaminase inhibitor, such as dCF, antiviral agent, antimetabolite agent, natural metabolite, nucleoside, nucleotide, purine base, pyrimidine base or an analog thereof (e.g., dAdo and cladribine), and the therapeutic formulations that include the adenosine deaminase inhibitor or other nucleoside analogue can be topically administered to a mammal to effectively treat a skin disorder, to effectively alleviate symptoms associated with a skin disorder, or a combination thereof. The adenosine deaminase inhibitor, such as dCF, antiviral agent, antimetabolite agent, natural metabolite, nucleoside, nucleotide, purine base, pyrimidine base or an analog thereof (e.g., dAdo and cladribine) described herein can be administered alone, but preferably is administered with a pharmaceutical carrier selected on the basis of the standard pharmaceutical practice.

One strategy to increase the safety margin of dCF, or another deaminase inhibitor or agent useful in the present inventions as described herein, is to limit exposure to just the epidermis and dermis. The dosage administered will, of course, vary depending upon known factors, such as the pharmacodynamic characteristics of the particular agent and its mode of administration; the age, health and weight of the recipient; the nature and extent of the symptoms; the kind of concurrent treatment; the frequency of treatment; and the effect desired.

The adenosine deaminase inhibitor, such as dCF, antiviral agent, antimetabolite agent, natural metabolite, nucleoside, nucleotide, purine base, pyrimidine base or an analog thereof (e.g., dAdo and cladribine) described herein, and the therapeutic formulations that include the adenosine deaminase inhibitor, such as dCF, antiviral agent, antimetabolite agent, natural metabolite, nucleoside, nucleotide, purine base, pyrimidine base or an analog thereof (e.g., dAdo and cladribine) described herein, can be topically administered as a lotion, cream, gel or ointment.

Experiments can be carried out to determine the concentration of topical dCF, or another deaminase inhibitor or agent useful in the present inventions as described herein, needed to induce apoptosis, and/or to inhibit proliferation of, monocytes, antigen presenting cells and/or lymphocytes in vivo, in situ or in vitro. Experiments conducted in vitro have generally included dAdo to synergize with dCF. Using the combination of drugs, effective cell killing by apoptosis usually takes 2-4 days. The degree of penetration of each drug into the epidermis and dermis is currently unknown, so formulations are being developed using a combination of empirical and inductive experiments. The concentration range for dCF and cladribine are guided by safety data from clinical studies in which leukemia patients and/or rheumatoid arthritis patients received IV dCF. Based upon in vitro and in vivo data, it is anticipated that dCF monotherapy may be effective, provided that endogenous (intradermal) dAdo accumulates during therapy. However, the effect may take many days to gain momentum. On the other hand, if dCF and dAdo are combined, either concurrently or sequentially, much lower levels of dCF may have activity, and the treatment duration may be reduced due to a more rapid rate of apoptosis of lymphocytes and monocytes within the dermis. The oncology dose is 4 mg/m2 every 14 days. (Grever et al., J. Clin. Oncol. 3:1196 (1985).) Based upon the assumption that the average body surface area is 1.8 m2, a safe dose of dCF is 7.2 mg every 14 days or 0.51 mg per day. For oncology patients, at this dosage, the adverse events were mild to moderate and diminished with treatment, therefore 4 mg/m2 is referred to as the safe dose that can be administered systemically. At this dose range, patients with hairy cell leukemia (HCL) achieve responses in the first few weeks, and complete remissions, on average, after 6 months of treatment. (Kraut et al., Blood, 68:1119 (1986).)

Based upon topical formulations of other drugs that are similar to dCF (e.g., cladribine) it is estimated that the systemic absorption of dCF will be no more than 7%. Calculations of safety margins have been performed, based upon this assumption and the known toxicity profile of dCF when administered IV for oncology indications. Systemic absorption is a concern, because of the potential for serious adverse events, such as lymphopenia and renal toxicity. At oncology doses, these adverse reactions are usually minimized, but the therapeutic index is narrow. Another factor that plays a role in the safety of the formulation is the amount of product administered each day. Approximately 4 grams, 12 grams, and 40 grams of a topical formulation are estimated to cover 10%, 30%, and 100% of the body surface area.

The examples herein illustrate safety margin calculations using dCF monotherapy, based upon about 1-20% (e.g., about 3-12% or about 0.5-5%) estimated fractional absorption, and various dose intensities.

Single and Combination Compositions and Treatment

dCF may be used as a topical single agent treatment for autoimmune skin diseases. Alternatively, dCF may be combined concurrently or sequentially with, e.g., dAdo to increase the apoptotic effects on lymphocytes, monocytes and possibly monocyte derived cells, such as macrophages and dendritic cells, such as Langerhans cells. The addition of dAdo elevates the level of dATP in lymphocytes and monocytes, leading to apoptosis via complex mechanisms. The combination of topical dCF and topical dAdo may enhance apoptosis of lymphocytes, monocytes, macrophages and dendritic cells in the dermis and epidermis, and it may also reduce the potential for systemic toxicity by limiting the lymphotoxic and monotoxic effects to the epidermis and dermis.

One strategy to increase the safety margin of dCF is to exploit the synergy between dCF and deoxyadenosine (dAd). These two agents are known to synergize in vitro, and it is believed that the anti-leukemic efficacy of dCF requires its cooperation with endogenous dAdo, which gradually accumulates in ADA sensitive cells during therapy. The invention described herein reveals novel compositions and uses that combine, e.g., dCF and dAdo to increase the therapeutic index of dCF.

In one embodiment, the two drugs are combined concurrently, to limit their effective pharmacological cooperation to the superficial epidermis and dermis—the anatomic compartment that harbors dermato-tropic autoimmune lymphocytes, and antigen-presenting cells such as monocytes, macrophages, and dendritic (Langerhans) cells. In a preferred embodiment, when applied as a topical combination therapy, effective therapeutic levels of the two drugs will be achieved only in the epidermis and dermis.

In another embodiment, the two topical agents are combined sequentially, to limit their effective pharmacological cooperation to the superficial epidermis and dermis. The latter strategy exploits the tight binding of dCF to ADA, which produces a sustained pharmacodynamic effect lasting nearly 3 days. The prolonged pharmacodynamic effect of dCF (i.e. extremely tight binding) is necessary for a sequential treatment method, because in order to achieve synergy, ADA must be inhibited or inactivated in intradermal lymphocytes, monocytes, macrophages and dendritic cells at the time dAdo is applied. By this time, most of the dCF that is not bound to the ADA enzyme has been absorbed from the dermis and epidermis and it has been cleared from circulation via the kidneys.

Other drugs have the potential to cooperate with dCF to induce apoptosis of lymphocytes and monocytes. For example, when used in combination with dCF, adenosine arabinoside (AraA) promotes the induction of apoptosis due to its ability to inhibit ribonucleotide reductase as well as DNA polymerases. By use of a concurrent topical application of dCF and AraA, the treatment duration may be reduced with an acceptable safety margin because the amount that is absorbed systemically is too low. An alternative strategy may be the sequential application of topical dCF followed by topical AraA. The synergy may provide a way to shorten the exposure time, and thus increase the therapeutic index. Thus, topical dCF inhibits ADA locally and the fraction of dCF that is absorbed systemically will be rapidly excreted. Next topical AraA is applied to potentiate the durable effect of dCF on the diseased skin. The fraction of AraA that is absorbed systemically will have minimal capacity to produce systemic toxicity, because the levels are below a critical threshold, and the absorbed dCF will have been cleared. The therapeutic index may be better than the concurrent application of topical dCF and topical AraA due to the separation in time. It is possible that the safety margins for both the concurrent and sequential combination regimens may be higher than dCF alone due to the shorter duration of treatment, and the possibility to use lower concentrations.

The invention provides several other agents that may be used in combination with dCF to potentiate the apoptotic activity of dCF towards intradermal lymphocytes, monocytes, macrophages and dendritic cells, and to reduce systemic toxicity. The drugs include, e.g., hydroxyurea, and other inhibitors of ribonucleotide reductase; and purine and pyrimidine nucleosides that directly or indirectly interfere with DNA biosynthesis.

A single topical formulation that contains both dCF and dAdo may decrease the time needed to achieve a response, because cells are killed more quickly in the presence of dAdo. The synergy between the two compounds is dependent on the concentration of each compound. The potential toxicity of combining dAdo and dCF has not been well defined in human studies. However, elevated levels of dAdo have been documented in the plasma of patients with hairy cell leukemia patients who have received dCF monotherapy. The examples herein assume effective penetration of both drugs in the dermis, and the range of concentrations for both dCF and dAdo are in excess of the concentrations known to be active against lymphocytes and/or monocytes in vitro. The ranges will be optimized empirically to obtain the desired efficacy, time to response and safety margin.

Deoxycoformycin is rapidly cleared from the plasma. When dCF is administered intravenously, it is cleared primarily by renal excretion with an elimination half-life of 3 to 9 hours. Approximately 96% of the drug is recovered in the urine. A fraction of deoxycoformycin is tightly bound to ADA. The binding complex of ADA-dCF has an extremely slow off-rate corresponding to a dissociation half-life of 68 hours. The sequential topical application of dCF exploits the rapid elimination of dCF and its tight binding to ADA. Thus, topical formulations of dCF and dAdo may be used sequentially to inhibit ADA (dCF) and to consummate the process of apoptosis induction (dAdo). The concept is to expose dermal and epidermal lymphocytes, monocytes, macrophages and dendritic cells to dCF so that ADA is inhibited. Then exposure to the drug is suspended, at which time the fraction of dCF that has been absorbed into the circulation is rapidly cleared from the tissues outside of the skin. At this time, dAdo is applied to the diseased skin, thereby triggering apoptosis of the ‘primed’ target cells within the dermis and epidermis. The dAdo is expected to be preferentially converted to dATP in the target immune cells within the diseased skin, because ADA is inhibited by the tightly bound dCF.

2′-Deoxyadenosine (dAdo) Potentiates the Effect of Deoxycoformycin (dCF)

2′-Deoxyadenosine (dAdo) is a purine nucleoside analogue that undergoes deamination by ADA to form 2′-deoxyinosine. Alternatively, it is phosphorylated by the enzyme, deoxycytidine kinase (dCK) to produce dAMP, dADP and dATP. In vitro, lymphocytes that are concurrently treated with dCF and dAdo are growth inhibited by 90-100% with the induction of apoptosis. The combination of dCF and dAdo inhibits DNA synthesis and also induces apoptosis in the target cells. In contrast, lymphocytes that are treated with the same concentration of dCF, in the absence of dAdo, are growth inhibited by 0-10% after 4 days of exposure.

It is believed that dAdo potentiates the growth inhibitory effect of dCF by up to 1,000-fold. Therefore, if endogenous pools of dAdo are not available within the dermis, the addition of dAdo may be necessary to generate a response. If endogenous concentrations of dAdo are too low, then exogenous dAdo may be added to a topical formulation to enhance the apoptotic effect of dCF. This may have important consequences that are particularly useful in the setting of topical dCF applications. First, it may reduce the time needed to achieve a response, because it obviates the need for endogenous dAdo to build up on the target tissue. Second, the combined use of these two drugs may lower the effective concentration of dCF. The reduced exposure time, taken together with a reduced concentration of dCF, may yield a better safety margin.

When dAdo is applied, it is estimated that no more than 7% will be absorbed into the systemic circulation. When diluted in the blood this quantity of dAdo is too low to produce synergy with the residual dCF that was not cleared from the body. However, dAdo is applied topically, thus synergy will exist locally in the dermis and epidermis, leading to apoptosis in the lymphocytes and monocytes in that anatomic compartment. With sequential application of dCF and dAdo, the lymphotoxic and monotoxic activity is maintained in the skin, while minimizing the potential for systemic toxicity. An example of sequential application is provided herein.

Sequential application of dCF and dAdo may have important consequences that are particularly useful in the setting of topical dCF applications. First, sequential application may reduce the time needed to achieve a response, because it obviates the need for endogenous dAdo to build up on the target tissue. Second, the combined use of these two drugs may lower the effective concentration of dCF. Third, by temporally separating the treatments, one avoids concurrent absorption of the two drugs, thereby reducing the risk of systemic toxicity. The reduced exposure time, temporal separation, and reduced concentration of dCF, may contribute to a better therapeutic index and a safer product.

Concurrent Treatment of dCF with AraA

The use of AraA in a concurrent treatment with dCF is based on a concept that is similar to the concurrent application of dCF and dAdo, although the mechanism of action of AraA and dAdo differ, as described above. The combination of AraA and dCF induces apoptosis and inhibits the growth of lymphocytes and monocytes at 100-fold lower concentrations than dAdo, and the same concentration of dCF. This higher sensitivity to the dCF/AraA combination may be exploited therapeutically. The synergistic effect may allow not only AraA concentrations to be lowered, but also the concentration of dCF.

Sequential Treatment of dCF with AraA

Sequential treatment of dCF/AraA is based upon concepts that are similar to the sequential treatment of dCF/dAdo. The treatment exploits the slow off-rate binding of dCF to ADA (half-life inhibition of 68 hours) and the fast clearance of dCF from the system.

Sequential application of dCF and dAraA may have important consequences that are particularly useful in the setting of topical dCF applications. First, sequential application may reduce the time needed to achieve a response, because it obviates the need for endogenous dAdo and dATP to build up in the target tissue. Second, the combined use of these two drugs may lower the effective concentration of dCF. Third, by temporally separating the treatments, one avoids concurrent absorption of the two drugs, thereby reducing the risk of systemic toxicity. The reduced exposure time, temporal separation, and reduced concentration of dCF, may contribute to a better therapeutic index and a safer product.

The present invention also provides other agents that can be used in combination with dCF to potentiate the apoptotic activity of dCF towards intradermal lymphocytes, monocytes, macrophages and dendritic cells, and to reduce systemic toxicity. The agents include: Hydroxyurea, and other inhibitors of ribonucleotide reductase, etc.; Purine nucleosides that directly or indirectly interfere with DNA biosynthesis (e.g., 6-thioguanine, 6-mercaptopurine, azathioprine, cladribine, fludarabine (preferably fludarabine des-phosphate), etc.); Pyrimidine nucleosides that directly or indirectly interfere with DNA biosynthesis (e.g., 5-fluorouracil, 5-fluorouridine, prodrugs of 5-fluorouridine, cytosine arabinoside, gemcitabine, dFdG, etc.); and inhibitors of viral nucleic acid metabolism, which have effects on host DNA biosynthesis metabolism and repair (e.g., ddI, AZT, lamivudine, ribavirin, imiquimod, abacavir, ganciclovir, acyclovir, valylciclovir, penciclovir, famciclovir, adefovir, tenofovir, cidofovir, trifluridine, vidarabine, etc).

Single Agent Treatment with dAdo

Based upon the surprising observation presented herein that dAd itself is able to induce apoptosis in U937 cells at relatively low concentrations (e.g., 100-200 μM), one embodiment of the present invention provides treatment of a skin disorder, such as an immune-mediated skin disorder/disease (e.g., psoriasis), including the administration of dAdo.

Single Agent Treatment with Cladribine

Another embodiment of the present invention provides treatment of a skin disorder, such as an immune-mediated skin disorder/disease (e.g., psoriasis), including the administration of cladribine.

Kits

Kits of the present invention will typically include one or more containers. The adenosine deaminase inhibitor and/or agent which is resistant to deamination (e.g., cladribine) and/or which inhibits DNA synthesis (directly or indirectly) will be present in one container. This container will typically be essentially free of liquid (e.g., will include less than about 10 wt. % liquid, less than about 1 wt. % liquid, less than about 0.5 wt. % liquid, or less than about 0.1 wt. % liquid). Specifically, this container will typically be essentially free of water (e.g., will include less than about 10 wt. % water, less than about 1 wt. % water, less than about 0.5 wt. % water, or less than about 0.1 wt. % water). If only one container is present, a pharmaceutically acceptable carrier will typically be present in that container. If a second container is present, a pharmaceutically acceptable carrier will typically be present in the second container.

Each of the containers can independently be a vial, syringe, etc. The containers can also optionally be configured to engage the other container(s), to facilitate effectively mixing and/or reconstituting the formulation prior to administration.

Such kits may further include, if desired, one or more of various conventional pharmaceutical kit components, such as for example, one or more pharmaceutically acceptable carriers, additional vials for mixing the components, etc., as will be readily apparent to those skilled in the art. Instructions, either as inserts or as labels, indicating quantities of the components to be administered, guidelines for administration, and/or guidelines for mixing the components, may also be included in the kit.

Coupling Syringe System

In the following description, for purposes of explanation, numerous specific details are set forth in order to provide a thorough understanding of the various embodiments. It will be apparent, however, to one skilled in the art that the various embodiments may be practiced without some of these specific details. The following description and drawings provide examples for illustration, but are not intended in a limiting sense and are not intended to provide an exhaustive treatment of all possible implementations.

It should be noted that references to “an”, “one”, or “various” embodiments in this disclosure are not necessarily to the same embodiment, and such references contemplate more than one embodiment.

The syringe system of the present invention includes two interlocking syringes in fluid-tight engagement for an effective mixing of a composition. The interlocking mechanism of the syringe system minimizes the loss of the mixed composition prior to administration. In addition, the syringe system is easily disassembled and configured to attach to a dispensing device for administration into or onto a patient.

The mixing of the composition to a desired consistency is easily achieved utilizing the syringe system. A first composition in the chamber of a first syringe is forced into the chamber of a second syringe containing a second composition by pushing a first syringe plunger from the proximal end of the first syringe toward the distal end of the first syringe. As the chamber of the second syringe fills with the composition from the first syringe, the pressure exerted on the fluid mixture in the chamber of the second syringe forces the second syringe plunger from the distal end of a second syringe toward the proximal end of the second syringe. Subsequently pushing the second syringe plunger from the proximal end of the second syringe toward the distal end of the second syringe fills the first chamber of the first syringe with the mixed composition from the second syringe. The pressure exerted on the fluid mixture in the chamber of the first syringe forces the first syringe plunger from the distal end of a first syringe toward the proximal end of the first syringe. This process is repeated until a desired consistency is achieved. A uniform composition consistency ensures the proper dosage of a medicament during administration to the patient.

Vial and syringe configurations in use today typically allow only a one-way transfer of one composition from the vial to the syringe containing another composition. A one-way transfer of the composition from the vial to the syringe does not provide the high integration of the compositions necessary for the proper administration of many medicaments. Similarly, manually shaking the composition in a syringe does not provide a uniform consistency compared with the forced fluid mixture of the syringe system.

The syringe system includes a first syringe having a female fitting and a first syringe barrel with an inner surface and an open proximal end. A first syringe plunger includes a first stopper tip in slidable communication with the inner surface of the first syringe barrel. The first syringe plunger is inserted into the open proximal end of the first syringe barrel. The first stopper tip is configured for fluid-tight engagement with a first composition located within a chamber at the distal end of the first syringe barrel.

The syringe system further includes a second syringe having a male fitting and a second syringe barrel with an inner surface and an open proximal end. A second syringe plunger includes a second stopper tip in slidable communication with the inner surface of the second syringe barrel. The second syringe plunger is inserted into the open proximal end of the second syringe barrel. The second stopper tip is configured for fluid-tight engagement with a second composition located within a chamber at the distal end of the second syringe barrel.

The female fitting of the first syringe is sized to receive and configured to interlock with the male fitting of the second syringe barrel for fluid-tight engagement of the compositions. The interlocking mechanism for fluid-tight engagement of the forced fluid mixture minimizes loss of the composition. Loss of the composition during mixing inhibits proper dosage as many drugs must be administered in a narrow dosage range. In addition, loss of the composition during mixing is costly as many medicaments are expensive and even a small amount of leakage is unacceptable.

In addition, the time between the mixing and the administration of the composition with the syringe system is minimal, such that a sensitive composition (i.e., a composition that, upon mixing, must be immediately administered) is not chemically or physically altered (i.e., there is minimal decomposition). Once a desired mix of the composition is achieved, the syringe system may be easily disassembled and configured to attach to an optional dispensing device for direct administration of the composition into and/or onto a patient.

In one embodiment, the syringe system is a syringe system kit that is pre-packaged for distribution. In one option, the syringe system kit includes labeling directly affixed and/or in proximity to the components of the kit. In another embodiment, the syringe system kit includes instructions or printed indicia.

The syringe system also includes a method for administering a composition mixture. In one embodiment, the method includes inserting at least one stopper tip inside the barrel of either or both the first and the second syringes. In another embodiment, the at least one stopper is pre-positioned in either or both the first and the second syringes. The user connects the first syringe with the female fitting to the second syringe with the male fitting. Each of the first and the second syringes independently contain a composition. The female fitting of the first syringe is interlocked with the male fitting of the second syringe for fluid-tight engagement of the mixed composition. The user forces at least a portion of the composition from the first syringe into the second syringe by a first syringe plunger, or alternatively, at least a portion of the composition from the second syringe into the first syringe by a second syringe plunger effective to provide a mixed composition. The first and the second syringes are subsequently disconnected from each other and an optional discharge assembly may be connected to at least one of the first and second syringes.

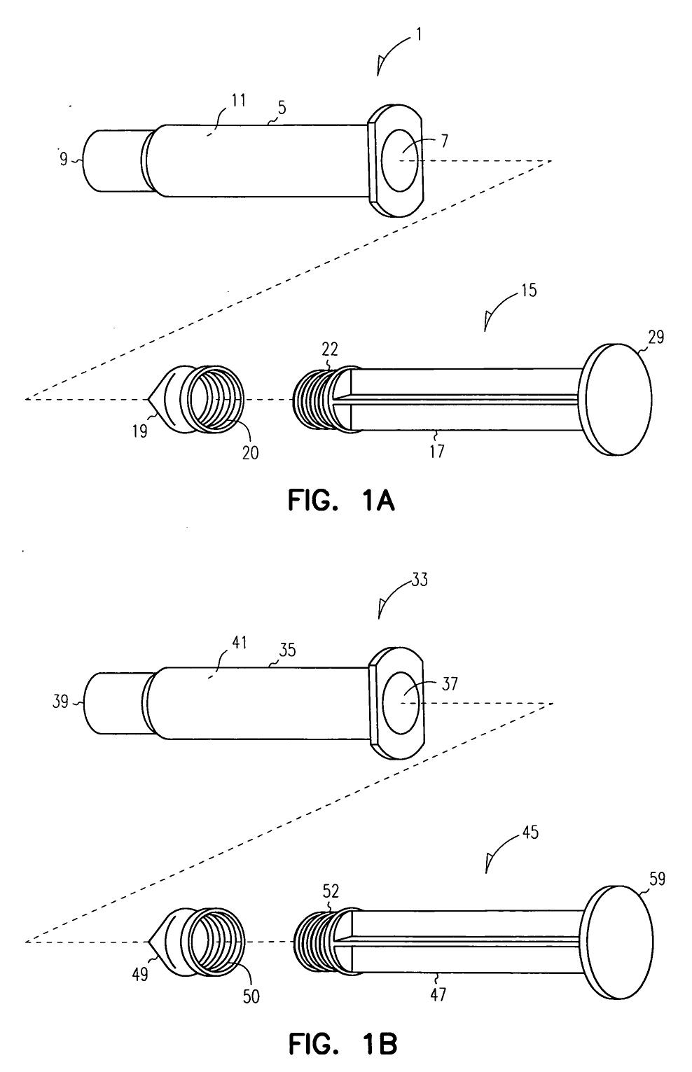

FIG. 1 illustrates one embodiment of a syringe 1 and a syringe 33. Syringes 1, 33 each include a syringe barrel 5, 35 having an open proximal end 7, 37, a distal end 9, 39 and a substantially cylindrical inner surface 11, 41. Plungers 15, 45 each include a plunger rod 17, 47 connected to a stopper tip 19, 49 and a plunger head 29, 59. In one embodiment, either or both of the first and the second stopper tips 19, 49 include a receptor 20, 50 configured to detachably engage an engager 22, 52 of either or both the first and the second syringe plungers 15, 45. In one embodiment, one or both receptors 20, 50 is a threaded receiving end and one or both engagers 22, 52 is a threaded protruding end. In another embodiment, one or both plunger rods 17, 47 are connected to the stopper tip 19, 49 by a snap-on locking configuration. In another embodiment, one or both plunger rods 17, 47 are connected to the stopper tip 19, 49 as one single piece.

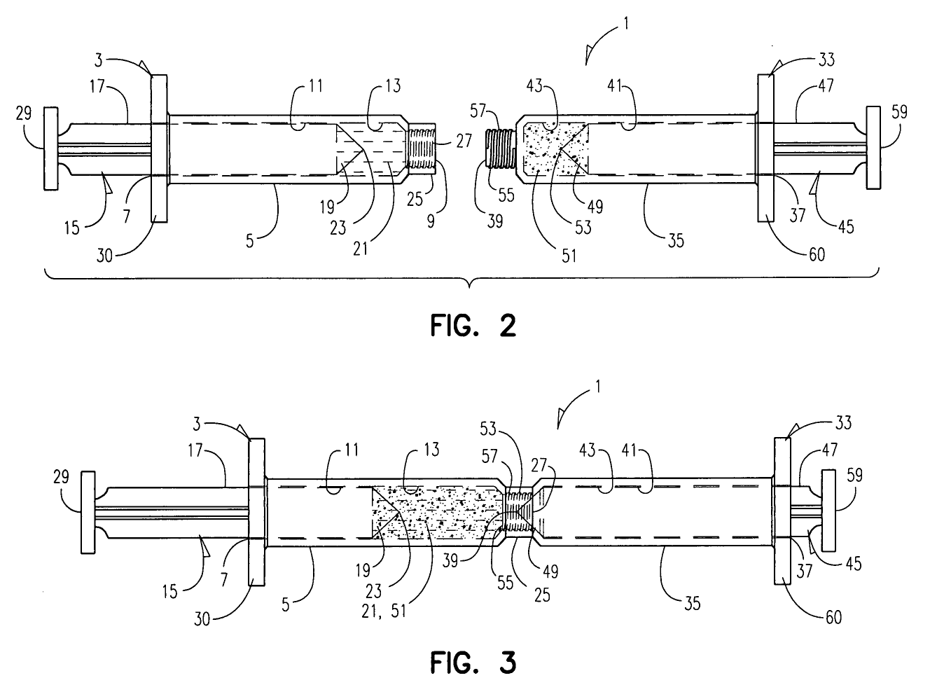

FIGS. 2 and 3 illustrate one embodiment of the syringe system 1. The syringe system 1 includes a first syringe 3 having a first syringe barrel 5 with an open proximal end 7, a distal end 9, and a substantially cylindrical inner surface 11 forming a chamber 13 extending therebetween. A first plunger 15 includes a plunger rod 17 connected to a first stopper tip 19 extending towards the distal end 9 of the first syringe barrel 5. The stopper tip 19 is slidably positioned into the cylindrical inner surface 11 through the proximal end 7 of the first syringe barrel 5 for maintaining fluid-tight engagement with the cylindrical inner surface 11 of the first syringe barrel 5.

In one embodiment, a composition 21 is introduced into the chamber 13 of the first syringe barrel 5 and displaced between the distal end 9 of the first syringe barrel 5 and a distal end 23 of the first stopper tip 19. The composition 21 includes a fluid, a solid, or a mixture thereof. The composition 21 includes, but is not limited to, a medication, a solution, or a combination thereof. In one option, the medication is lyophilized. In another option, the solution is a diluent. Distal end 9 of the first syringe barrel 5 includes a female fitting 25 that extends axially there through and communicates with the chamber 13 of the syringe barrel 5. The female fitting 25 includes a threaded receiving end 27.

A second syringe 33 includes a second syringe barrel 35 having an open proximal end 37, a distal end 39, and a substantially cylindrical inner surface 41 forming a chamber 43 extending therebetween. A second plunger 45 includes a plunger rod 47 connected to a second stopper tip 49 extending towards the distal end 39 of the second syringe barrel 35. The stopper tip 49 is slidably positioned into the cylindrical inner surface 41 through the proximal end 37 of the second syringe barrel 35 for maintaining fluid-tight engagement with the cylindrical inner surface 41 of the second syringe barrel 35.

In one embodiment, a composition 51 is introduced into the chamber 41 of the second syringe barrel 35 and displaced between the distal end 39 of the second syringe barrel 35 and the distal end 53 of the second stopper tip 49. The composition 51 includes a fluid, a solid, or a mixture thereof. The composition 51 includes, but is not limited to, a medication, a solution, or a combination thereof. In one option, the medication is lyophilized. In another option, the solution is a diluent. The distal end 39 of the second syringe barrel 35 includes a male fitting 55 that extends axially there through and communicates with the chamber 41 of the second syringe barrel 35. The male fitting 55 includes a threaded end 57.

In one embodiment, either or both of the first and the second syringes 3, 33 independently include an outwardly projecting flange 30, 60 near the proximal end 7, 37 of the first and the second syringes 3, 33. The flange 30, 60 provides a gripping means for the first and the second syringes 3, 33 when pushing the plunger 15, 45 along the inner surface 11, 41 of the first and the second syringe barrels 5, 35.

In one embodiment, the first syringe 3 is disengageably interlocked with the second syringe 33. The threaded receiving end 27 of the female fitting 25 of the first syringe barrel 5 is mated with the threaded end 57 of the male fitting 55 of the second syringe barrel 35 by connecting the threaded receiving end 27 of the female fitting 25 with the threaded end 57 of the male fitting 55 and turning the threaded receiving end 27, the threaded end 57, or both in a locked position for fluid-tight engagement.

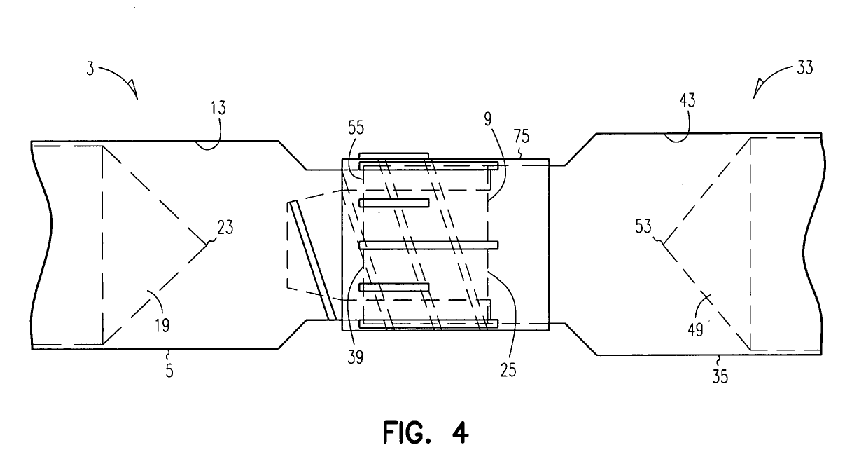

FIG. 4 illustrates one embodiment of the female fitting 25 of the first syringe 3 disengageably interlocked to the male fitting 55 of the second syringe 33 via a locking ring 75 (depicted in FIG. 4). In one option, the locking ring 75 is threadingly coupled about an exterior surface of the second syringe 33. In another option, the locking ring 75 is rotatably coupled with the male fitting 55 and is threadingly coupled with one or more projections disposed on an outer surface of the female fitting 25. In one embodiment, the male/female interlocking mechanism is a luer-lock. In another embodiment, the male/female interlocking mechanism is a snap-lock.

Once the first syringe 3 is interlocked with the second syringe 33, the composition 21 located in the chamber 13 of the first syringe barrel 5 is ready for mixture with the composition 51 located in the chamber 43 of the second syringe barrel 35. Mixture of the compositions 21, 51 is achieved by the alternating fluid-tight movement of the first stopper tip 19 by the first plunger 15 sliding along the cylindrical inner surface 11 of the first syringe barrel 5 and the second stopper tip 49 by the second plunger 45 sliding along the cylindrical inner surface 41 of the second syringe barrel 35.

The alternating fluid-tight movement between the chamber 13 of the first syringe barrel 5 and the chamber 43 of the second syringe barrel 35 is achieved by pushing a plunger head 29 connected to the first plunger rod 17 of the plunger 15 which forcibly pushes the distal end 23 of the interconnected stopper tip 19 along the cylindrical inner surface 11 toward the distal end 9 of the first syringe barrel 5. The sliding motion of the stopper tip 19 by the first plunger 15 toward the distal end 9 of the first syringe barrel 5 forces the composition 21 from the chamber 13 of the first syringe barrel 5 to the chamber 43 of the second syringe barrel 35 combining composition 21 with composition 51. As the chamber 43 of the second syringe barrel 35 fills with the composition 21, the pressure exerted on the composition mixture 21, 51 in the chamber 43 of the second syringe 33 pushes the stopper tip 49 back toward the proximal end 37 of the second syringe barrel 35 pushing the plunger 45 distally and away from the proximal end 37 of the second syringe barrel 35.

Subsequently pushing the plunger head 59 of the second plunger 45 toward the distal end 39 of the second barrel 35 forcibly pushes the distal end 53 of the interconnected stopper tip 49 along the cylindrical inner surface 41 toward the distal end 39 of the second syringe barrel 35. The sliding motion of the stopper tip 49 toward the distal end 39 of the second syringe barrel 5 forces the composition mixture 21, 51 from the chamber 43 of the second syringe barrel 35 back through to the chamber 13 of the first syringe barrel 5. The sliding motion of the stopper tip 49 by the second plunger 45 toward the distal end 39 of the first syringe barrel 35 forces the composition mixture 21, 51 from the chamber 43 of the second syringe barrel 35 to the chamber 13 of the first syringe barrel 5 remixing the composition mixture 21, 51. As the chamber 13 of the first syringe barrel 5 fills with the composition mixture 21, 51, the pressure exerted on the composition mixture 21, 51 in the chamber 13 of the first syringe 3 pushes the stopper tip 19 back toward the proximal end 7 of the first syringe barrel 5 subsequently pushing the plunger 15 distally and away from the proximal end 7 of the first syringe barrel 5. The alternating movement of pushing and pulling the first plunger 15 of the first syringe 3 and second plunger 45 of the second syringe 33 is repeated to achieve a uniform mixture of the composition 21, 51.

The first and second stopper tips 19, 49 include any suitable shape, provided that the first and second stopper tips 19, 49 maintain fluid-tight engagement with the inner surface 11, 41 of the first and the second syringe barrels 5, 35. The distal end 23, 53 of the first and second stopper tips 19, 49 are shaped to facilitate the egress of the composition mixture 21, 51 from the chamber 13, 43 of the first and the second syringe barrels 5, 35. In one embodiment, the cross-sectional configuration of the distal end 23, 53 of the first and second stoppers 19, 49 are v-shaped.

In one embodiment, the first stopper tip 19 and/or the second stopper tip 49 include a plurality of annular ribs dimensioned for maintaining fluid-tight engagement when sliding within the inner surface 11, 41 of the first and the second syringe barrels 5, 35. In one embodiment the stopper tips 19, 49 include 2 to 3 annular ribs. In another embodiment, the stopper tips 19, 49 include 4 to 5 annular ribs.

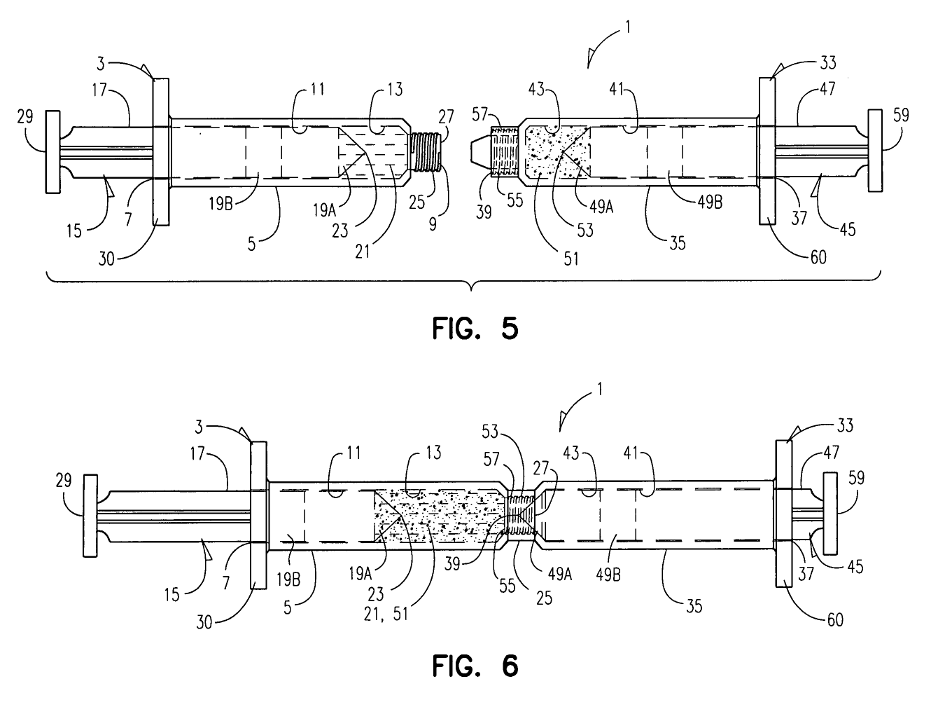

FIGS. 5 and 6 illustrate one embodiment of a coupling syringe system 1. The syringe system 1 includes a first and second syringe 3, 33 having a first and second syringe barrel 5, 35 with an open proximal end 7, 37, a distal end 9, 39 and a substantially cylindrical inner surface 11, 41 forming a chamber 13, 43 extending therebetween. Distal end 9 of the first syringe barrel 5 includes a female fitting 25 that extends axially there through and communicates with the chamber 13 of the syringe barrel 5. Distal end 39 of the second syringe barrel 35 includes a male fitting 55 that extends axially there through and communicates with the chamber 43 of the syringe barrel 35.

In one embodiment, either or both of the first and the second syringes 3, 33 include a secondary stopper tip 19B, 49B disposed between a primary stopper tip 19A, 49A and the proximal end 7, 37 of the second syringe barrels 5, 35. The secondary stopper tips 19B, 49B assist in keeping the inner surface 11, 41 of the first and second syringe barrels 5, 35 sterile prior to packaging. Typically, the compositions 21, 51 are displaced between the distal end 9, 39 of first and second syringe barrels 5, 35, which is sealed, and the stopper tips 19, 49, which create the seal. As such, the compositions 21, 51 are usually contained within the sterile chambers 13, 43 of the first and second syringe barrels 5, 35. The portion of the first and second syringe barrels 5, 35 between the plunger tips 19, 49 and the proximal end 7, 37, however, is open to the environment. Even though the first and second syringes 3, 33 may be packaged in a sterile packaging system, non-sterile matter (e.g., bacteria) can be introduced in that portion of the first and second syringe barrels 5, 35 during packaging and can survive (i.e., remain dormant) in the first and second syringe barrels 5, 35 over a lengthy storage time. In one embodiment, one of the compositions 21, 51 is a lyophilized pharmaceutical. Reconstitution of the lyophilized pharmaceutical can introduce non-sterile matter (e.g., bacteria) present on the inner surface 11, 41 of the first and second syringe barrels 5, 35. This occurs because the plunger rod 17, 47 and the stopper tips 19, 49 may be drawn back and forth along that portion of the inner surface 11, 41 of the first and second syringe barrels 5, 35 where non-sterile matter was introduced. Each cycling of the stopper tip 19, 49 along the inner surface 11, 41 of the first and second syringe barrels 5, 35 provide potential for contamination of the contents contained within the first and second syringe syringes 3, 33.

The secondary stopper tip 19B, 49B disposed between the primary stopper tip 19A, 49A and the proximal end 7, 37 of the first and second syringe barrels 5, 35 provide a seal of that portion of the first and second syringe barrels 5, 35 between the plunger tips 19, 49 and the proximal end 7, 37 no longer exposing the inner surface 11, 41 of the first and second syringe barrels 5, 35 to the environment.

In one embodiment, the first plunger 15 includes the plunger rod 17 connected to the secondary stopper tip 19B disposed between the primary stopper tip 19A and the proximal end 7 of the first syringe barrel 5. The positioning of the secondary stopper tip 19B toward the proximal end 7 of the first syringe barrel 5 and the primary stopper tip 19A toward the distal end 9 of the first syringe barrel 5 within the cylindrical inner surface 11 encapsulates air between the primary stopper tip 19A and the secondary stopper tip 19B. The plunger rod 17 engages the secondary stopper tip 19B to facilitate operation of secondary stopper tip 19B within the cylindrical inner surface 11 of the first syringe barrel 5.

A second syringe 33 includes a second syringe barrel 35 having an open proximal end 37, a distal end 39, and a substantially cylindrical inner surface 41 forming a chamber 43 extending therebetween. Distal end 39 of the second syringe barrel 35 includes a male fitting 55 that extends axially there through and communicates with the chamber 43 of the second syringe barrel 35.

A second plunger 45 includes a plunger rod 47 connected to a secondary stopper tip 49B disposed between a primary stopper tip 49A and the proximal end 37 of the second syringe barrel 35. The positioning of the secondary stopper tip 49B toward the proximal end 37 of the second syringe barrel 35 and the primary stopper tip 49A toward the distal end 39 of the second syringe barrel 35 within the cylindrical inner surface 11 encapsulates air between primary stopper tip 49A and secondary stopper tip 49B. In one embodiment, the plunger rod 47 engages the secondary stopper tip 49B to facilitate operation of the secondary stopper tip 49B within the cylindrical inner surface 11 of the second syringe barrel 35.

In one embodiment, a composition 21 is introduced into the chamber 13 of the first syringe barrel 5 and displaced between the distal end 9 of the first syringe barrel 5 and the distal end 23 of the primary stopper tip 19A. In another embodiment, a composition 51 is introduced into the chamber 41 of the second syringe barrel 35 and displaced between the distal end 39 of the second syringe barrel 35 and the distal end 53 of the primary stopper tip 49A. The compositions 21 and 51 include, but are not limited to, a medication, a solution, or a combination thereof In one option, the medication is lyophilized. In another option, the solution is a diluent.

In one embodiment, the first syringe 3 is disengageably interlocked with the second syringe 33. The threaded receiving end 27 of the female fitting 25 of the first syringe barrel 5 disengagably interlocked with the threaded end 57 of the male fitting 55 of the second syringe barrel 35 by connecting the threaded receiving end 27 of the female fitting 25 with the threaded end 57 of the male fitting 55 and turning the threaded receiving end 27, the threaded end 57, or both in a locked position for fluid-tight engagement.

In one embodiment, the female fitting 25 of the first syringe 3 is disengageably interlocked to the male fitting 55 of the second syringe 33 via a locking ring 75. In one option, the locking ring 75 is threadingly coupled about an exterior surface of the second syringe 33. In another option, the locking ring 75 is rotatably coupled with the male fitting 55 and is threadingly coupled with one or more projections disposed on an outer surface of the female fitting 25. In one embodiment, the male/female interlocking mechanism is a luer-lock. In another embodiment, the male/female interlocking mechanism is a snap-lock.

Once the first syringe 3 is interlocked with the second syringe 33, the composition 21 located at the distal end 9 of the first syringe barrel 5 is ready for mixture with the composition 51 located at the distal end 39 of the second syringe barrel 35. Mixture of the compositions 21, 51 is achieved by the alternating fluid-tight movement of the first stopper tip 19A sliding along the first cylindrical inner surface 11 of the first syringe barrel 5 and the first stopper tip 49A sliding along the cylindrical inner surface 41 of the second syringe barrel 35.

The alternating fluid-tight movement between the chamber 13 of the first syringe barrel 5 and the chamber 43 of the second syringe barrel 35 is achieved by pushing a plunger head 29 connected to the plunger rod 17 of the plunger 15 which forcibly pushes the interconnected secondary stopper tip 19B toward the distal end 9 of the first syringe barrel 5. As the secondary stopper tip 19B slides along the inner surface 11 toward the distal end 9 of the first syringe barrel 5, the encapsulated air between the primary stopper tip 19A and the secondary stopper tip 19B is compressed forcing the primary stopper tip 19A toward the distal end 9 of the first syringe barrel 5.

The sliding motion of the primary stopper tip 19A toward the distal end 9 of the first syringe barrel 5 forces the composition 21 from the chamber 13 of the first syringe barrel 5 to the chamber 43 of the second syringe barrel 35 combining composition 21 with composition 51. As the chamber 43 of the second syringe barrel 35 fills with the composition 21, the pressure exerted on the composition mixture 21, 51 in the chamber 43 of the second syringe 33 pushes the stopper tip 49A back toward the proximal end 37 of the second syringe barrel 35 which in turn compresses the encapsulated air between the primary stopper tip 49A and the secondary stopper tip 49B forcing both the primary stopper tip 49A and the secondary stopper tip 49B toward the proximal end 37 of the second syringe barrel 35. The sliding motion of the secondary stopper tip 49B toward the proximal end 37 of the second syringe barrel 35 pushes the plunger 45 distally and away from the proximal end 37 of the second syringe barrel 35.

Subsequently pushing the plunger head 59 of the second plunger 45 toward the distal end 39 of the second syringe barrel 35 forcibly pushes the interconnected secondary stopper tip 49B toward the distal end 39 of the second syringe barrel 35. As the secondary stopper tip 19B slides along the inner surface 41 toward the distal end 39 of the second syringe barrel 35, the encapsulated air between the primary stopper tip 49A and the secondary stopper tip 49B is compressed forcing both the primary stopper tip 49A toward the distal end 39 of the second syringe barrel 35.

The sliding motion of the primary stopper tip 49A toward the distal end 39 of the second syringe barrel 35 forces the composition mixture 21, 51 from the chamber 43 of the second syringe barrel 35 to the chamber 13 of the first syringe barrel 5 combining composition 21 with composition 51. As the chamber 13 of the first syringe barrel 5 fills with the composition mixture 21, 51, the pressure exerted on the composition mixture 21, 51 in the chamber 13 of the first syringe 3 pushes the stopper tip 19A back toward the proximal end 7 of the first syringe barrel 5 which in turn compresses the encapsulated air between the primary stopper tip 19A and the secondary stopper tip 19B forcing both the primary stopper tip 19A and the secondary stopper tip 19B toward the proximal end 7 of the first syringe barrel 5. The sliding motion of the secondary stopper tip 19B toward the proximal end 7 of the first syringe barrel 5 pushes the plunger 15 distally and away from the proximal end 7 of the second syringe barrel 5. The alternating movement of pushing and pulling the first plunger 15 of the first syringe 3 and second plunger 45 of the second syringe 33 is repeated to achieve a uniform mixture of the composition mixture 21, 51.

Manufacture of the Syringe System

The first and second syringes 3, 33 are manufactured from any suitable material. In one embodiment, the first and second syringes 3, 33 are manufactured from glass. In another embodiment, the first and second syringes 3,33 are manufactured from plastic. The plastics used in the manufacture of the first and second syringes 3, 33 include, but are not limited to, polypropylene, polyethylene, polycarbonate and polystyrene. In another embodiment, the first and second syringes 3, 33 are manufactured from thermoplastic elastomers.

In one embodiment, the stopper tips 19, 49 are manufactured from rubber. The rubber used to manufacture the stopper tips 19, 49 include, but are not limited to, natural rubber and synthetic rubber.

In one embodiment, the first and the second syringes 3, 33 are manufactured by an injecting molding process where each syringe is made as one unit. In another embodiment, the second syringe 33 is manufactured by independently molding the second syringe 33 and locking ring 75 and then mounting (i.e., attaching) the locking ring 75 to the second syringe 33. In one option, the locking ring 75 is permanently attached to the second syringe 33 by welding the two pieces together.

In one embodiment, the first and second syringe barrels 5, 35 are sterilized prior to packaging. In one option, the barrels 5, 35 are sterilized by gamma irradiation. In one embodiment, the sterilization of the barrels 5, 35 occur before the one or more of the compositions 21, 51 are introduced into the chambers 13, 43 of the first and second syringe barrels 5, 35.

The size of the first and second syringe barrels 5, 35 are each independently any suitable size. In one embodiment, the syringe barrel 5, 35 is about 0.01 cc to about 100 cc. In another embodiment, the syringe barrel 5, 35 is about 0.1 cc to about 50 cc. In yet another embodiment, the syringe barrel 5, 35 is about 0.1 cc to about 25 cc. In still yet another embodiment, the syringe barrel 5, 35 is about 0.5 cc to about 10 cc.

### ENUMERATED EMBODIMENTS

The present invention provides for the following enumerated embodiments:
[1] The present invention provides for a topical composition that includes a topical carrier and an adenosine deaminase inhibitor.
[2] The present invention also provides for the topical composition of embodiment [1], wherein the topical carrier is a cream, lotion, gel or ointment.
[3] The present invention also provides for the topical composition of any one of embodiments [1]-[2], wherein the adenosine deaminase inhibitor is isolated from Streptomyces antiobiotiucus, Aspergillus nidulans; or is synthetically prepared.
[4] The present invention also provides for the topical composition of any one of embodiments [1]-[3], wherein the adenosine deaminase inhibitor is selected from the group of cladribine, deoxycoformycin (pentostatin), coformycin, diethyl pyrocarbonate, erythro-9-(2-hydroxy-3-nonyl) adenine, erythro-9-[3-(2-hydroxynonyl)]adenosine, erythro-9-(2-hydroxy-3-nonyl)-adenosine (EHNA), 6-(R)-hydroxyl-1,6-dihydropurine ribonucleoside (HDPR), imidazole-4-carboxamide derivatives, erythro-6-amino-9(2-hydroxy-3-nonyl)-purine hydrochloride, erythro-9-(2-hydroxy-3-nonyl)-3-deazaadenine, 1-deazaadenosine, Adenosine, 2-cyano-2′,3′-dideoxy-, Adenosine, 2′,3′-dideoxy-2-ethyl-, Adenosine, 2′,3′-dideoxy-2-(methylthio)-, Adenosine, 2′,3′-dideoxy-2-(trifluoromethyl)-, 2′,3′-Dideoxy-2-iodoadenosine, (+/−)-9H-Purine-9-ethanol, 6-amino-β-hexyl-α-methyl-, and analogs and combinations thereof.
[5] The present invention also provides for the topical composition of any one of embodiments [1]-[4], wherein the adenosine deaminase inhibitor is (R)-3-(2-Deoxy-β-D-erythro-pentofuranosyl)-3,6,7,8-tetrahydroimidazo[4,5-d][1,3]diazepin-8-ol.
[6] The present invention also provides for the topical composition of any one of embodiments [1]-[5], wherein the adenosine deaminase inhibitor is cladribine.
[6b] The present invention also provides for the topical composition of any one of embodiments [1]-[5], wherein the adenosine deaminase inhibitor is a combination of at least two of deoxycoformycin (dCF), deoxyadenosine (dAdo), cladrabine (CdA), fludarabine (F-Ara-A), cytrabine (Ara-C), and thioguanine.
[7] The present invention also provides for a topical composition that includes a topical carrier and cladribine.
[8] The present invention also provides for a pharmaceutical composition that includes: (1) a pharmaceutically acceptable carrier, (2) an adenosine nucleotide, an adenosine nucleoside, an adenosine base, or an analog thereof, and (3) a skin penetration enhancer.
[9] The present invention also provides for the pharmaceutical composition of embodiment [8], wherein the skin penetration enhancer is selected from the group consisting of DMSO, EDTA, EGTA, Loramide DEA, Ethoxydiglycol, NMP, Triacetin, Propylene Glycol, Benzyl Alcohol, Sodium Laureth Sulfate, Dimethyl Isosorbide, Isopropyl Myristate, Olive Squalane, Medium Chain Triglyceride Oil (MCT Oil), Menthol, Isopropyl Palmitate, Isopropyl Isostearate, Propylene Glycol Monostearate, Lecithin, Diisopropyl Adipate, Diethyl Sebacate, Oleic Acid, Ethyl Oleate, Urea, Glyceryl Oleate, Caprylic/Capric Triglyceride, Propylene Glycol Dicaprylate/Dicaprate, Laureth 4, Oleth -2, Oleth-20, Propylene Carbonate, Nonoxynol-9, 2-n-nonyl-1,3-dioxolane, C7 to C14-hydrocarbyl substituted 1,3-dioxolane, 1,3-dioxane, or acetal, and Nonoxynol-15.
[10] The present invention also provides for the pharmaceutical composition of any one of embodiments [8]-[9], wherein the pharmaceutically acceptable carrier is a skin penetration enhancer.
[11] The present invention also provides for the pharmaceutical composition of any one of embodiments [8]-[10], wherein the adenosine nucleotide, adenosine nucleoside, adenosine base, or analog thereof is 2′-deoxyadenosine (dAd), cladribine or adenosine arabinoside (AraA).
[12] The present invention also provides for the pharmaceutical composition of any one of embodiments [8]-[11], wherein the adenosine nucleotide, adenosine nucleoside, adenosine base, or analog thereof is 2′-deoxyadenosine (dAd).
[13] The present invention also provides for the pharmaceutical composition of any one of embodiments [8]-[12], wherein the adenosine nucleotide, adenosine nucleoside, adenosine base, or analog thereof is cladribine.
[14] The present invention also provides for the pharmaceutical composition of any one of embodiments [8]-[3], wherein the carrier is a topical carrier.
[15] The present invention also provides for the pharmaceutical composition of any one of embodiments [8]-[14], wherein the topical carrier is a cream, lotion, gel or ointment.
[16] The present invention also provides for a pharmaceutical composition that includes: (1) a pharmaceutically acceptable carrier, (2) 2-deoxyadenosine (dAdo), and (3) a skin penetration enhancer.
[17] The present invention also provides for a pharmaceutical composition including: (1) a pharmaceutically acceptable carrier, (2) cladribine, and (3) a skin penetration enhancer.
[18] The present invention also provides for the pharmaceutical composition of embodiment [16] or [17], wherein the pharmaceutically acceptable carrier is a skin penetration enhancer.
[19] The present invention also provides for a pharmaceutical composition that includes: (1) a pharmaceutically acceptable carrier, (2) an adenosine deaminase inhibitor, and (3) an adenosine nucleotide, an adenosine nucleoside, an adenosine base, or an analog thereof.
[20] The present invention also provides for the pharmaceutical composition of embodiment [ 19], wherein the adenosine deaminase inhibitor is isolated from Streptomyces antiobiotiucus, Aspergillus nidulans; or is synthetically prepared.
[21] The present invention also provides for the pharmaceutical composition of any one of embodiments [19]-[20], wherein the adenosine deaminase inhibitor is selected from the group of cladribine, deoxycoformycin (pentostatin), coformycin, diethyl pyrocarbonate, erythro-9-(2-hydroxy-3-nonyl) adenine, erythro-9-[3-(2-hydroxynonyl)]adenosine, erythro-9-(2-hydroxy-3-nonyl)-adenosine (EHNA), 6-(R)-hydroxyl-1,6-dihydropurine ribonucleoside (HDPR), imidazole4-carboxamide derivatives, erythro-6-amino-9(2-hydroxy-3-nonyl)-purine hydrochloride, erythro-9-(2-hydroxy-3-nonyl)-3-deazaadenine, 1-deazaadenosine, Adenosine, 2-cyano-2′,3′-dideoxy-, Adenosine, 2′,3′-dideoxy-2-ethyl-, Adenosine, 2′,3′-dideoxy-2-(methylthio)-, Adenosine, 2′,3′-dideoxy-2-(trifluoromethyl)-, 2′,3′-Dideoxy-2-iodoadenosine, (+/−)-9H-Purine-9-ethanol, 6-amino-β-hexyl-α-methyl-, and analogs and combinations thereof.
[22] The present invention also provides for the pharmaceutical composition of any one of embodiments [19]-[21], wherein the adenosine deaminase inhibitor is (R)-3-(2-Deoxy-β-D-erythro-pentofuranosyl)-3,6,7,8-tetrahydroimidazo[4,5-d][1,3]diazepin-8-ol.
[23] The present invention also provides for the pharmaceutical composition of any one of embodiments [19]-[22], wherein the adenosine nucleotide, adenosine nucleoside, adenosine base, or analog thereof is deoxyadenosine (dAdo), cladribine or adenosine arabinoside (AraA).
[24] The present invention also provides for the pharmaceutical composition of any one of embodiments [1]-[23], further including a topical antipsoriatic agent.
[25] The present invention also provides for the pharmaceutical composition of any one of embodiments [1]-[24], further including a biological agent to treat psoriasis.
[26] The present invention also provides for the pharmaceutical composition of any one of embodiments [1]-[25], further including at least one of an antimicrobial preservative, an emulsifying agent, a solubilizing agent, a humectant, an ointment base, a solvent, a stiffening agent, a viscosity-inducing agent, a wetting agent, mineral oil, propylene glycol, wax, natural oil, synthetic oil, a skin absorption enhancer, a keratolytic agent, a topical moisturizer, an analgesic, an anesthetic, an antipruritic and water.
[27] The present invention also provides for the pharmaceutical composition of any one of embodiments [1]-[26], further including a corticosteroid, calcipotriene, a retinoid, anthralin, coal tar, salicylic acid, photochemotherapy with ultraviolet A (PUVA), phototherapy with UVB, or a combination thereof.
[28] The present invention also provides for the pharmaceutical composition of any one of embodiments [1], [3]-[14], and [16]-[27] wherein the composition is formulated in a gel, ointment, lotion or cream.
[29] The present invention also provides for a method for treating a skin disorder in a mammal inflicted with a skin disorder, the method including topically administering, to a mammal in need of such treatment, a pharmaceutical composition of any one of embodiments [1]-[28] in an amount effective to treat the skin disorder.
[30] The present invention also provides for a method for alleviating a symptom associated with a skin disorder in a mammal inflicted with a skin disorder, the method including topically administering, to a mammal afflicted with a skin disorder, a pharmaceutical composition of any one of embodiments [1]-[28] in an amount effective to alleviate the symptom associated with the skin disorder.
[31] The present invention also provides for the method of any one of embodiments [29] or [30], wherein the skin disorder is mediated by an immunological response, mechanism, or process.
[32] The present invention also provides for the method of embodiments [29] or [30], wherein the skin disorder is a lymphocyte-mediated skin disorder.
[33] The present invention also provides for the method of embodiments [29] or [30], wherein the skin disorder is selected from the group of acne, eczema, psoriasis, rosacea, skin cancer, skin bums, skin allergies, congenital skin disorders, acantholysis, acanthosis, acanthosis nigricans, dermatosis, disease, erythroderma, furunculosis, impetigo, jungle rot, keratoderma, keratodermia, keratonosis, keratosis, keratosis nigricans, leukoderma, lichen, livedo, lupus, melanism, melanosis, molluscum, necrobiosis lipoidica, necrobiosis lipoidica diabeticorum, pemphigus, prurigo, rhagades, Saint Anthony's fire, seborrhea, vitiligo, xanthoma, xanthosis, Psoriatic arthritis, Reiter's syndrome, Guttate psoriasis, Dyshidriotic eczema, Acute and chronic graft versus host disease, Systemic sclerosis, Morphea, Spongiotic dermatitis, Allergic dermatitis, Nummular eczema, Pityriasis rosacea, Pityriasis rubra pilaris, Pemphigus erythematosus, Pemphigus vulgaris, Lichenoid keratosis, Lichenoid nitidus, Lichen planus, Lichenoid dermatitis, Seborrheic dermatitis, Autosensitization dermatitis, Dermatitis herpetiformis, and Eosinophilic dermatitis.
[34] The present invention also provides for the method of embodiments [29] or [30], wherein the skin disorder is a chronic skin disorder.
[35] The present invention also provides for the method of embodiment [30], wherein the symptom is selected from the group of pain, itching, inflammation, and combinations thereof.
[36] The present invention also provides for the method of any one of embodiments [29]-[35], wherein the administration occurs after the mammal has achieved a remission following treatment with a biological therapy.
[36b] The present invention also provides for the method of any one of embodiments [29]-[35], wherein the administration of the adenosine deaminase inhibitor is a co-administration of at least two of deoxycoformycin (dCF), deoxyadenosine (dAdo), cladrabine (CdA), fludarabine (F-Ara-A), cytrabine (Ara-C), and thioguanine.
[36c] The present invention also provides for the method of embodiment [36b], wherein the co-administration is sequential.
[36d] The present invention also provides for the method of embodiment [36b], wherein the co-administration is simultaneous.
[37] The present invention also provides for a kit that includes:

a first container including a pharmaceutical composition of any one of embodiments [1]-[28] or a combination thereof that is essentially free of liquid; and
a second container including a pharmaceutically acceptable carrier.
[38] The present invention also provides for a kit of embodiment [37], wherein the second container further including a pharmaceutical composition of any one of embodiments [1]-[28].
[39] The present invention also provides for a kit of any one of embodiments [37]-[38], wherein the liquid is water.
[40] The present invention also provides for a kit of any one of embodiments [37]-[39], further including instructions or printed indicia.
[41] The present invention also provides for a kit of any one of embodiments [37]-[40], wherein the pharmaceutical composition exists as a solid.
[42] The present invention also provides for a kit of any one of embodiments [37]-[41], wherein the pharmaceutical composition exists as a lyophilized solid.
[43] The present invention also provides for a kit of any one of embodiments [37]-[42], wherein the first container includes less than about 5 wt. % liquid, based upon the weight of the pharmaceutical composition.
[44] The present invention also provides for a kit of any one of embodiments [37]-[43], wherein the first container includes less than about 1 wt. % liquid, based upon the weight of the pharmaceutical composition.
[45] The present invention also provides for a kit of any one of embodiments [37]-[44], wherein the first container includes less than about 0.5 wt. % liquid, based upon the weight of the pharmaceutical composition.
[46] The present invention also provides for a kit of any one of embodiments [37]-[45], wherein less than about 5 wt. % of the pharmaceutical composition decomposes at a temperature of about 65° F. to about 75° F., over a period of time of up to about 60 days.
[47] The present invention also provides for a kit of any one of embodiments [37]-[46], wherein less than about 1 wt. % of the pharmaceutical composition decomposes at a temperature of about 65° F. to about 75° F., over a period of time of up to about 60 days.
[48] The present invention also provides for a kit of any one of embodiments [37]-[47], wherein less than about 0.5 wt. % of the pharmaceutical composition decomposes at a temperature of about 65° F. to about 75° F., over a period of time of up to about 60 days.
[49] The present invention also provides for a kit of any one of embodiments [37]-[48], wherein the first container is a syringe.
[50] The present invention also provides for a kit of any one of embodiments [37]-[49], wherein the second container is a syringe.
[51] The present invention also provides for a kit of any one of embodiments [37]-[50], wherein the first container is a syringe, the second container is a syringe, and the two syringes are adapted to reversibly interconnect in fluid tight engagement with each other.
[52] The present invention also provides for a syringe system that includes: a first syringe having a female fitting, the first syringe including a first syringe barrel having an inner surface and an open proximal end;
   a first syringe plunger having a first stopper tip in slidable communication with the inner surface of the first syringe barrel via the open proximal end, the first stopper tip configured for fluid-tight engagement with a first composition;
   a second syringe having a male fitting, the second syringe including a second syringe barrel having an inner surface and an open proximal end; and
   a second syringe plunger having a second stopper tip in slidable communication with the inner surface of the second syringe barrel via the open proximal end, the second stopper tip configured for fluid-tight engagement with a second composition;
   wherein the female fitting is sized to receive and configured to interlock with the male fitting for fluid-tight engagement between the first and the second syringes;
   the first syringe containing a pharmaceutical composition of any one of embodiments [1]-[28] and the second syringe containing a carrier and/or a second pharmaceutical composition of any one of embodiments [1]-[28].
[53] The present invention also provides for the syringe system of embodiment [52], wherein the female fitting is sized to receive and configured to interlock with the male fitting by a locking ring.
[54] The present invention also provides for the syringe system of embodiment [53], wherein the locking ring is rotatably coupled with the male fitting and the locking ring is threadingly coupled with one or more projections disposed on an outer surface of the female fitting.
[55] The present invention also provides for the syringe system of embodiment [52], wherein either or both the female fitting and the male fitting are configured to detachably connect to a discharge assembly.
[56] The present invention also provides for the syringe system of embodiment [52], wherein a secondary stopper tip is disposed between a primary stopper tip and the proximal end of either or both the first and the second syringe barrels.
[57] The present invention also provides for the syringe system of embodiment [52], further including an outwardly projecting flange near the proximal end of either or both the first syringe and the second syringe.
[58] The present invention also provides for the syringe system of embodiment [52], wherein each syringe barrel independently including a volume from about 0.01 cc to about 100 cc.
[59] The present invention also provides for the syringe system of embodiment [52], wherein each syringe barrel independently including a volume from about 0.5 cc to about 10 cc.
[60] The present invention also provides for the syringe system of embodiment [52], wherein either or both the first and the second stopper tips are detachably engaged to either or both the first and the second syringe plungers.
[61] The present invention also provides for a method for topically administering a mixed composition, which includes:
   connecting a first syringe having a female fitting to a second syringe having a male fitting, the first and the second syringes each independently containing a composition;
   interlocking the female fitting of the first syringe to the male fitting of the second syringe for fluid-tight engagement;
   the first syringe containing a pharmaceutical composition of any one of any one of embodiments [1]-[28] and the second syringe containing a carrier and/or a second pharmaceutical composition of any one of embodiments [1]-[28];
   forcing at least a portion of the composition from the first syringe into the second syringe by a first syringe plunger or forcing at least a portion of the composition from the second syringe into the first syringe by a second syringe plunger, effective to provide a mixed composition;
   disconnecting the first and the second syringes; and

forcing at least a portion of the mixed composition through the syringe effective to administer the mixed composition to the patient.
[62] The present invention also provides for the method of embodiment [61], wherein interlocking the female fitting of the first syringe to the male fitting of the second syringe includes interlocking the female fitting of the first syringe to the male fitting of the second syringe by a locking ring.
[63] The present invention also provides for the method of embodiment [61], further including connecting at least one discharge assembly to at least one of the first and second syringes.
[64] The present invention also provides for the method of embodiment [63], wherein connecting at least one discharge assembly to at least one of the first and second syringes includes connecting at least one discharge assembly to the male fitting of the second syringe by a locking ring.
[65] The present invention also provides for the method of embodiment [61], further including inserting at least one stopper tip inside the barrel of either or both the first and the second syringes.
[66] The present invention also provides for the method of embodiment [61], wherein providing a mixed composition includes alternately forcing at least a portion of the composition from the first syringe into the second syringe by the first syringe plunger or forcing at least a portion of the composition from the second syringe into the first syringe by the second syringe plunger.
[67] The present invention also provides for a syringe system kit, which includes:
   a first syringe having a female fitting, the first syringe including a first syringe barrel having an inner surface and an open proximal end;
   a first syringe plunger having a first stopper tip in slidable communication with the inner surface of the first syringe barrel via the open proximal end, the first stopper tip configured for fluid-tight engagement with a first composition;
   a second syringe having a male fitting, the second syringe including a second syringe barrel having an inner surface and an open proximal end; and
   a second syringe plunger having a second stopper tip in slidable communication with the inner surface of the second syringe barrel via the open proximal end, the second stopper tip configured for fluid-tight engagement with a second composition; and
   wherein the female fitting is sized to receive and configured to interlock with the male fitting for fluid-tight engagement between the first and the second syringes;
   the first syringe containing a pharmaceutical composition of any one of embodiments [1]-[28] and the second syringe containing a carrier and/or a second pharmaceutical composition of any one of embodiments [1]-[28].
[68] The present invention also provides for the kit of embodiment [67], further including instructions or printed indicia.
[69] The present invention also provides for the kit of embodiment [67], further including labeling directly affixed and/or in proximity to the components of the kit.
[70] The present invention also provides for the kit of any one of embodiments [67]-[69], further including a discharge assembly.
[71] The present invention also provides for the kit of any one of embodiments [67]-[69], wherein the syringe system is pre-packaged.
[72] The present invention also provides for a two-part delivery system which includes:
   a first container including a pharmaceutical composition of any one of embodiments [1]-[28], that is essentially free of liquid; and
   a second container including a pharmaceutically acceptable carrier.
[73] The present invention also provides for a two-part delivery system of embodiment [72], wherein the first container further includes a second pharmaceutical composition of any one of embodiments [1]-[28].
[74] The present invention also provides for a two-part delivery system of embodiment [72], wherein the second container further includes a pharmaceutical composition of any one of embodiments [1]-[28].
[75] The present invention also provides for a two-part delivery system of any one of embodiments [72]-[74], wherein the liquid is water.
[76] The present invention also provides for a two-part delivery system of any one of embodiments [72]-[75], wherein the pharmaceutically acceptable carrier is a gel, ointment, lotion, or cream.
[77] The present invention also provides for a two-part delivery system of any one of embodiments [72]-[76], further including instructions or printed indicia.
[78] The present invention also provides for a two-part delivery system of any one of embodiments [72]-[77], wherein the pharmaceutical composition exists as a solid.
[79] The present invention also provides for a two-part delivery system of any one of embodiments [72]-[78], wherein the pharmaceutical composition exists as a lyophilized solid.
[80] The present invention also provides for a two-part delivery system of any one of embodiments [72]-[79], wherein the first container includes less than about 5 wt. % liquid, based upon the weight of the pharmaceutical composition.
[81] The present invention also provides for a two-part delivery system of any one of embodiments [72]-[80], wherein the first container includes less than about 1 wt. % liquid, based upon the weight of the pharmaceutical composition.
[82] The present invention also provides for a two-part delivery system of any one of embodiments [72]-[81], wherein the first container includes less than about 0.5 wt. % liquid, based upon the weight of the pharmaceutical composition.
[83] The present invention also provides for a two-part delivery system of any one of embodiments [72]-[82], wherein less than about 5 wt. % of the pharmaceutical composition decomposes at a temperature of about 65° F. to about 75° F., over a period of time of up to about 60 days.
[84] The present invention also provides for a two-part delivery system of any one of embodiments [72]-[83], wherein less than about 1 wt. % of the pharmaceutical composition decomposes at a temperature of about 65° F. to about 75° F., over a period of time of up to about 60 days.
[85] The present invention also provides for a two-part delivery system of any one of embodiments [72]-[84], wherein less than about 0.5 wt. % of the pharmaceutical composition decomposes at a temperature of about 65° F. to about 75° F., over a period of time of up to about 60 days.
[86] The present invention also provides for a two-part delivery system of any one of embodiments [72]-[85], wherein the first container is a syringe.
[87] The present invention also provides for a two-part delivery system of any one of embodiments [72]-[86], wherein the second container is a syringe.
[88] The present invention also provides for a two-part delivery system of any one of embodiments [72]-[87], wherein the first container is a syringe, the second container is a syringe, and the two syringes are adapted to reversibly interconnect in fluid tight engagement with each other.
[89] The present invention also provides a pharmaceutical composition of any one of embodiments [1]-[28] for use in medical therapy or diagnosis.
[90] The present invention also provides the use of a pharmaceutical composition of any one of embodiments [1]-[28] for the manufacture of a medicament for treating a topical skin disorder.
[91] The present invention also provides for the use of a pharmaceutical composition of any one of embodiments [1]-[28], for the manufacture of a medicament for alleviating a symptom associated with a skin disorder in a mammal inflicted with a skin disorder.
[92] The present invention also provides for the use of a pharmaceutical composition of any one of embodiments [90] or [91], wherein the skin disorder is mediated by an immunological response, mechanism, or process.
[93] The present invention also provides for the use of a pharmaceutical composition of any one of embodiments [90] or [91], wherein the skin disorder is a lymphocyte-mediated skin disorder.
[94] The present invention also provides for the use of a pharmaceutical composition of any one of embodiments [90] or [91], wherein the skin disorder is selected from the group of acne, eczema, psoriasis, rosacea, skin cancer, skin burns, skin allergies, congenital skin disorders, acantholysis, acanthosis, acanthosis nigricans, dermatosis, disease, erythroderma, furunculosis, impetigo, jungle rot, keratoderma, keratodermia, keratonosis, keratosis, keratosis nigricans, leukoderma, lichen, livedo, lupus, melanism, melanosis, molluscum, necrobiosis lipoidica, necrobiosis lipoidica diabeticorum, pemphigus, prurigo, rhagades, Saint Anthony's fire, seborrhea, vitiligo, xanthoma, xanthosis, Psoriatic arthritis, Reiter's syndrome, Guttate psoriasis, Dyshidriotic eczema, Acute and chronic graft versus host disease, Systemic sclerosis, Morphea, Spongiotic dermatitis, Allergic dermatitis, Nummular eczema, Pityriasis rosacea, Pityriasis rubra pilaris, Pemphigus erythematosus, Pemphigus vulgaris, Lichenoid keratosis, Lichenoid nitidus, Lichen planus, Lichenoid dermatitis, Seborrheic dermatitis, Autosensitization dermatitis, Dermatitis herpetiformis, and Eosinophilic dermatitis.
[95] The present invention also provides for the use of a pharmaceutical composition of any one of embodiments [90] or [91], wherein the skin disorder is a chronic skin disorder.
[96] The present invention also provides for the use of a pharmaceutical composition of embodiment [91], wherein the symptom is selected from the group of pain, itching, inflammation, and combinations thereof.
[97] The present invention also provides for the use of a pharmaceutical composition of any one of embodiments [90]-[96], wherein the administration occurs after the mammal has achieved a remission following treatment with a biological therapy.

This description has set forth numerous characteristics and advantages of various embodiments and details of structure and function of various embodiments, but is intended to be illustrative and not intended in an exclusive or exhaustive sense. Changes in detail, material and management of parts, order of process and design may occur without departing from the scope of the appended claims and their legal equivalents. Obviously, numerous modifications and variations of the present invention are possible in light of the above teachings. It is therefore to be understood that within the scope of the appended claims, the invention may be practiced otherwise than as specifically described herein.

The invention can be illustrated with the following non-limiting examples:

### EXAMPLES

The following prophetic examples, falling within the scope of the invention, can be carried out.

### Example 1

Apply a topical formulation of dCF in the concentration range of about 0.00001 to about 0.01 w/w % to the inflamed areas, with or without an adhesive skin patch, at 100% of the BSA, for a period of 1 day to 6 months. The number of concurrent treatments could be up to as many as 3 treatments per day for a period of up to about 12 weeks. After each treatment the inflamed area may be washed with soap and water and dried before the next application.

Using the highest concentration of 0.01% (0.10 mg/g), 100% BSA coverage (40 grams) and 7% systemic absorption, patients would be systemically exposed to 0.28 mg of dCF per day. This corresponds to a safety margin of 1.8 times more safe than the oncology treatment. For the subsequent lower concentrations, the safety factor would be 18, 180, and 1800.

### Example 2

Apply a topical formulation of dCF in the concentration range of about 0.00001 to about 0.01 w/w % to the inflamed areas, with or without an adhesive skin patch, at 30% of the BSA, for a period of 1 day to 6 months. The number of concurrent treatments could be up to as 3 treatments per day for a period of up to about 12 weeks. After each treatment the inflamed area may be washed with soap and water and dried before the next application.

Using the highest concentration of 0.01% (0.10 mg/g), 30% BSA coverage (12 grams) and 7% systemic absorption, patients would be systemically exposed to 0.084 mg of dCF per day. This corresponds to a safety margin of 6. For the subsequent lower concentrations, the safety factor would be 60, 600, and 6000.

### Example 3

Apply a topical formulation of dCF in the concentration range of 0.00001-0.10 w/w % to the inflamed areas, with or without an adhesive skin patch, at 10% of the body surface area, for period of 1 day to 6 months. The frequency of applications could be up 3 times per day. After each treatment the inflamed area may be washed with soap and water and dried before the next application.

Using the highest concentration of 0.10% (1.0 mg/g), 10% body surface area coverage (4 grams) and 7% systemic absorption, patients would be systemically exposed to 0.028 mg of dCF per day. This corresponds to a safety margin of 1.8. For the subsequent lower concentrations, the safety factor would be 18, 180, 1800, and 18000.

The dCF concentrations proposed above are in the concentration range to inhibit the growth of lymphocytes in vitro, but that is in combination with dAdo. Assuming that the intracellular levels of dAdo are sufficient to generate elevated endogenous levels of dATP, the above examples will be adequate to treat patients with autoimmune diseases. From the above examples, at the most severe case where 40 grams of a 0.01% dCF topical formulation is delivered per day, this treatment would still be 1.8 times safer than the oncology dose. For most patients with mild to moderate topical autoimmune conditions, the application would be less than 12 grams per day. At 12 grams per day this treatment would be 6 times safer than the oncology dose. To improve the therapeutic index, the treatment schedule could be extended and lower concentrations administered thereby increasing the safety margins in the range of 50 to 18000.

### Example 4

Solution Formulation:

| Component | Function | Amount (% w/w) |
|---|---|---|
| 2′-Deoxycoformycin | Active | 0.00001-0.10 |
| Isopropyl Alcohol | Solvent | 35-55 |
| Propylene Glycol | Solvent | 1-15 |
| Hydroxypropyl Cellulose | Thickening agent | 0-5 |
| Phosphoric Acid | Acidifying Agent | pH = 7-9 |
| Dibasic sodium phosphate | Base | 0.01-1.5 |
| Menthol | Odorant | 0-1 |
| Purified Water | Diluent | 25-80 |

### Example 5

Gel Formulation:

| Component | Function | Amount (% w/w) |
|---|---|---|
| 2′-Deoxycoformycin | Active | 0.00001-0.10 |
| Propylene glycol | Solvent | 0.1-10 |
| Methylparaben | Preservative | 0.01-0.1 |
| Propylparaben | Preservative | 0.01-0.1 |
| Edetate Disodium | Chelating agent | 0.01-0.1 |
| Dibasic sodium phosphate | Basic Agent | 0.01-1.5 |
| Carbomer | Gelling Agent | 0.1-2 |
| Phosphoric Acid | Neutralizing Agent | QS pH 7-9 |
| Ethanol | Solvent | 0-75 |
| Purified Water | Solvent | 25-95 |

### Example 6

Cream Formulation:

| Component | Function | Amount (% w/w) |
|---|---|---|
| 2′-Deoxycoformycin | Active | 0.00001-0.10 |
| Sorbitol 70% Solution | Humectant | 1-3 |
| Emulsifying Wax | Cream Base | 5-25 |
| Glycerin | Emollient | 0-20 |
| Isopropyl Palmitate | Penetration | 1-10 |
| Benzyl alcohol | Preservative | 0.1-0.5 |
| Edetate disodium | Chelating agent | 0.01-0.55 |
| Dibasic sodium phosphate | Basic Agent | 0.01-0.55 |
| Phosphoric Acid | Neutralizing Agent | pH 7-9 |
| Ceteth 20 | Surfactant | 0-5 |
| Mineral Oil | Emollient | 0-55 |
| Purified Water | Solvent | 50-80 |

### Example 7

Ointment Formulation:

| Excipient | Function | Amount (% w/w) |
|---|---|---|
| 2′-Deoxycoformycin | Active | 0.00001-0.10 |
| Microcrystalline Wax | Ointment base | 1-15 |
| White Petrolatum | Ointment base | 55-99 |
| Tocopherol | Anti-oxidant | 0-0.5 |
| Steareth-2 | Surfactant | 1-10 |
| Propylene Glycol | Solvent | 1-10 |
| Edetate disodium | Chelating agent | 0.001-0.55 |
| Dibasic sodium phosphate | Basic Agent | 0.01-0.55 |
| Phosphoric Acid | Neutralizing Agent | pH 7-9 |
| Purified Water | Solvent | 1-10 |

### Example 8

Two-Part Formulation:

Nipent, a commercial oncology injectable product has a two-year shelf life and it is supplied as a lyophilized powder. Given the stability of dCF when lyophilized, a two-part formulation would consist of dCF lyophilized in Container A (e.g., a syringe) and a topical vehicle in Container B (e.g., a syringe). Prior to administration, the dCF and delivery components are mixed thoroughly, for example, by repetitively transferring the components between the syringes. In this way, the product is reconstituted.

A two-part (mix and use) formulation minimizes the probability of dCF hydrolysis because the drug is exposed to water only very briefly prior to application. The preferred two-part formulation uses a cream base; however, a gel or ointment base may also be implemented if necessary. An example of a two-part cream formulation is listed below.

### Example 9

Two-Part Cream Formulation:
Part 1 - Drug solution for lyophilization: Container/Syringe A:

| Component | Function | Amount (% w/v) |
|---|---|---|
| 2′-Deoxycoformycin | Active | 0.000026-0.26 |
| Mannitol | Bulking Agent | 0.0-8.0 |
| Polyethylene Glycol 4000 | Bulking Agent | 0.0-8.0 |
| Water for Injection | Solvent | QS 100 |

Fill Target = 0.4 mL in a 3-cc Becton Dickinson Sterifill Syringe
Final Drug Amount = 0.05 mg

Part 2 - Cream Base for reconstitution: Container/Syringe B:

| Component | Function | Amount (% w/w) |
|---|---|---|
| Sorbitol 70% Solution | Humectant | 1.0-5.0 |
| Emulsifying Wax | Cream Base | 5.0-20.0 |
| Glycerin | Emollient | 1.0-5.0 |
| Isopropyl Palmitate | Penetration | 2.0-10.0 |
| Benzyl alcohol | Preservative | 0.1-0.5 |
| Dibasic sodium phosphate | Basic Agent | 0.1-0.5 |
| Phosphoric Acid | Neutralizing Agent | QS pH = 7-9 |
| Purified Water | Solvent | QS 100 |

Fill Target = 1 g in a 3-cc female Ultratek Syringe

Final Reconstituted Product:

| Component | Function | Amount (% w/w) |
|---|---|---|
| 2′-Deoxycoformycin | Active | 0.00001-0.10 |
| Mannitol | Bulking Agent | 0-21 |
| Polyethylene Glycol 4000 | Bulking Agent | 0-21 |
| Sorbitol 70% Solution | Humectant | 1-5 |
| Emulsifying Wax | Cream Base | 5-20 |
| Glycerin | Emollient | 1-5 |
| Isopropyl Palmitate | Penetration | 2-10 |
| Benzyl alcohol | Preservative | 0.1-0.5 |
| Dibasic sodium phosphate | Basic Agent | 0.01-0.10 |
| Phosphoric Acid | Neutralizing Agent | QS pH = 7-9 |
| Purified Water | Solvent | QS 100 |

Reconstitution: For example, Syringe A and Syringe B are coupled
together by means of integrated Leur Loks. The product is mixed by first
passing the Cream Base into the Drug Syringe, and then returning the
mixture to Syringe B. The product is mixed back and forth in this manner
30-100 times to completely mix the drug with the cream. The cream
formulation is then dispensed from the Syringe A for application.

### Example 10

Apply a topical formulation that contains dCF in the concentration range of about 0.00001 to about 0.01 w/w % and dAdo in the concentration range of about 0.00005 to about 5.0 w/w % to the inflamed areas, with or without an adhesive skin patch. The number of concurrent treatments could be as little as 1 and as many as 3 treatments per day for a period of up to about 12 weeks. Treatment of up to 100% of BSA will be used.

### Example 11

Step 1. Apply a topical formulation that contains dCF in the concentration range of about 0.00005 to about 0.1 w/w % with or without an occlusive dressing. The number of treatments with dCF could be about 1 to about 14 days (e.g., about 1 to about 7 days). After each administration the inflamed area may be washed with soap and water and dried before next application. Treatment area is up to 20% of BSA. The treatment will give dCF adequate time to inhibit the ADA enzyme in cells that are in the dermis. After washing the treated areas, the application area should be dried thoroughly before next application to eliminate the possible hydrolysis of dCF.

Step 2. Apply a topical formulation that contains dAdo in the concentration range of about 0.00005 to about 5.0 w/w % with or without an occlusive dressing. The number of treatments with dAdo could be about 1 to about 14 days (e.g., about 1 to about 7 days). After each administration the inflamed area may be washed with soap and water and dried before next application. Treatment area is up to 100% of BSA. At this point ADA is inhibited in the treated cells so dAdo will not be deaminated in those cells. This will allow dAdo to preferentially be converted to dATP generating the elevated levels of dATP necessary to induce apoptosis.

Step 3. After Step 1 and 2 are completed, over the next 0-5 days no treatment will occur. This time will be used to monitor the affect of the treatment and give time for the system to equilibrate before the start of the next sequential treatment.

Step 4. If the desired response is not obtained, Steps 1-3 will be repeated for a period of up to 12-weeks.

### Example 12

Step 1. Apply a topical formulation that contains dCF in the concentration range of about 0.00005 to about 0.01 w/w % with or without an occlusive dressing. The number of treatments with dCF could be about 1 to about 14 days (e.g., about 1 to about 7 days). After each administration the inflamed area may be washed with soap and water and dried before next application. Treatment area is up to 100% of BSA. The treatment will give dCF adequate time to inhibit the ADA enzyme in cells that are in the dermis. After washing the treated areas, the application area should be dried thoroughly before next application to eliminate the possible hydrolysis of dCF.

Step 2. Apply a topical formulation that contains dAdo in the concentration range of about 0.00005 to about 5.0 w/w % with or without an occlusive dressing. The number of treatments with dAdo could be about 1 to about 14 days (e.g., about 1 to about 7 days). After each administration the inflamed area may be washed with soap and water and dried before next application. Treatment area is up to 100% of BSA. At this point ADA is inhibited in the treated cells so dAdo will not be deaminated in those cells. This will allow dAdo to preferentially be converted to dATP generating the elevated levels of dATP necessary to induce apoptosis.

Step 3. After Step 1 and 2 are completed, over the next 0-5 days no treatment will occur. This time will be used to monitor the affect of the treatment and give time for the system to equilibrate before the start of the next sequential treatment.

Step 4. If the desired response is not obtained, Steps 1-3 will be repeated for a period of up to 12-weeks.

### Example 13

Apply a topical formulation that contains dCF in the concentration range of 0.00005-0.10 w/w % and dAdo in the concentration range of 0.00005-5.0% (w/w) to the inflamed areas with or without an adhesive skin patch. The frequency of applications may be once per day and as many as 3 times per day over a period of 6 months. Usually a topical formulation containing dCF and dAdo will be applied to no more than 20% of the body surface area; however, in patients with more severe disease, the application may include in excess of 75% of the body surface area.

### Example 14

Sequential Topical Application of dCF, Followed by Topical Application of dAdo

Step 1. Apply a topical formulation that contains dCF in the concentration range of 0.00005-0.10 w/w % with or without an occlusive dressing. The frequency of applications with dAdo may be about 1 to about 14 days. After each administration, the inflamed skin may be washed and dried before next application. The treatment area will usually not exceed 20% of the body surface area, but in patients with severe disease, it may exceed 75% of the body surface area. The treatment will give dCF adequate time to inhibit the ADA enzyme in cells that are in the dermis. After washing the treated areas, the application area should be dried thoroughly before next application to eliminate the possible hydrolysis of dCF.

Step 2. Apply a topical formulation that contains dAdo in the concentration range of 0.00005-5.0 w/w % with or without an occlusive dressing. The frequency of applications with dAdo may be about 1 to about 14 days. After each administration, the inflamed skin may be washed and dried before next application. The treatment area will usually not exceed 20% of the body surface area, but in patients with severe disease, it may exceed 75% of the body surface area.

Step 3. If the desired response is not obtained, Steps 1-3 will be repeated for a period of up to 6 months. Each sequence of dCF/dAdo applications is a treatment cycle. Treatment cycles may range for 2 days to one month. Many treatment cycles may be implemented until a response is achieved.

### Example 15

Apply a topical formulation that contains dCF in the concentration range of about 0.00001 to about 0.01 w/w % and AraA in the concentration range of about 0.0000005 to about 5.0 w/w % to the inflamed areas, with or without an adhesive skin patch. The number of concurrent treatments could be as little as 1 and as many as 3 treatments per day for a period of up to about 12 weeks. Treatment of up to 100% of BSA will be used.

### Example 16

Apply a topical formulation that contains dCF in the concentration range of about 0.00005 to about 0.01 w/w % and AraA in the concentration range of about 0.0000005 to about 5.0 w/w % to the inflamed areas, with or without an adhesive skin patch. The number of concurrent treatments could be as little as 1 and as many as 3 treatments per day for a period of up to about 12 weeks. Treatment of up to 100% of BSA will be used.

### Example 17

Step 1. Apply a topical formulation that contains dCF in the concentration range of about 0.00001 to about 0.10 w/w % with or without an occlusive dressing. The number of treatments with dCF could be about 1 to about 14 days (e.g., about 1 to about 7 days). After each administration the inflamed area may be washed with soap and water and dried before next application. Treatment area is up to 20% of BSA. The treatment will give dCF adequate time to inhibit the ADA enzyme in cells that are in the dermis. After washing the treated areas, the application area should be dried thoroughly before next application to eliminate the possible hydrolysis of dCF.

Step 2. Apply a topical formulation that contains AraA in the concentration range of about 0.00005 to about 5.0 w/w % with or without an occlusive dressing. The number of treatments with AraA could be as little as 1 and as many as 10 from 1-5 days. After each administration the inflamed area may be washed with soap and water and dried before next application. Treatment area is up to 100% of BSA. At this point ADA is inhibited in the treated cells so AraA will not be deaminated in those cells. This will allow AraA to preferentially be converted to Ara-ATP, inhibit DNA synthesis, and induce apoptosis.

Step 3. After Step 1 and 2 are completed, over the next 0-5 days no treatment will occur. This time will be used to monitor the affect of the treatment and give time for the system to equilibrate before the start of the next sequential treatment.

Step 4. If the desired response is not obtained, Steps 1-3 will be repeated for a period of up to about 12-weeks.

### Example 18

Concurrent Treatment of dCF with AraA

Apply a topical formulation that contains dCF in the concentration range of 0.00005-0.10 w/w % and AraA in the concentration range of 0.0000005-0.10 w/w % to the inflamed areas, with or without an adhesive skin patch. The frequency of applications may be from 1 to 3 times per day for a period of up to 6 months. Typically the treatment includes less than 20% of the body surface area, but it may exceed 75% of body surface area.

### Example 19

Sequential Treatment of dCF with AraA

Step 1. Apply a topical formulation that contains dCF in the concentration range of 0.00005-0.10 w/w % with or without an occlusive dressing. The frequency of applications with dAdo may be between 1 and 3 times per day for 1-14 days. After each administration the inflamed skin may be washed and dried before next application. The treatment area will usually not exceed 20% of the body surface area, but in patients with severe disease, it may exceed 75% of the body surface area.

Step 2. Apply a topical formulation that contains AraA in the concentration range of 0.00005-5.0 w/w % with or without an occlusive dressing. The frequency of applications with dAdo may be about 1 to about 14 days. After each administration the inflamed area may be washed and dried before next application. The treatment area will usually not exceed 20% of the body surface area, but in patients with severe disease, it may exceed 75% of the body surface area.

Step 3. If the desired response is not obtained, Steps 1-3 will be repeated for a period of up to 6 months. Each sequence of dCF/dAraA applications is a treatment cycle. Treatment cycles may range for 2 days to one month. Many treatment cycles may be implemented until a response is achieved.

### Example 20

Concurrent Treatment of dCF with dAd

Apply a topical formulation that contains dCF in the concentration range of 0.00005-0.10 w/w % and dAd in the concentration range of 0.00005-5.0 w/w % to the inflamed areas, with or without an adhesive skin patch. The frequency of applications may be from 1 to 3 times per day for a period of up to 6 months. Typically the treatment includes less than 20% of the body surface area, but it may exceed 75% of body surface area.

### Example 21

Sequential Exposure of T-cells and Monocytoid Cells to Deoxycoformycin (dCF) and Deoxyadenosine (dAd)

Introduction

Deoxycoformycin (dCF) is known to induce apoptosis in T-cells and monocytoid cells through the inhibition of adenosine deaminase (ADA). The concurrent exposure of the cells to dCF and deoxyadenosine (dAd) leads to greater levels of apoptosis than dCF alone; and the addition of dAd reduces the effective dose of dCF required to induce apoptosis (Niitsu et al. 1999; Niitsu et al. 2000; Bagnara, et al. 1992; for the latter reference see below). In other words, dAd potentiates the pro-apoptotic effect of dCF on T-cells and monocytoid cells.

A sequential exposure experiment has not been conducted before. Note that Bagnara et al. (1992) added dCF and then added dAd to human T-lymphoblasts. The interval between addition of dCF and dAd was 30 minutes. These investigators did not wash out the dCF before adding the dAd. Therefore, the cells were exposed to dCF alone and subsequently to both agents. Thus, this is a concurrent, not a sequential, exposure experiment.

Deoxycoformycin (dCF) binds extremely avidly to ADA with a dissociation half-life of 68 hours. Thus, it is hypothesized that the sequential exposure of the cells to dCF, followed by deoxyadenosine (dAd) will lead to greater levels of apoptosis than dCF alone. Whether cells in which ADA is inhibited by dCF are susceptible to the induction of apoptosis by dAd (after dCF has been washed out from the medium) was investigated. To test this hypothesis, cells were exposed to dCF for 2 days, washed the cells to remove free dCF from the cell culture medium, and then incubated the cells for another two days. The concentration of dAd needed to induce apoptosis in T cells (Jurkat) and monocytoid (U937) cells that have been exposed to dCF was determined.

Methods

Jurkat T cells and the U937 monocytoid cells were cultured in the presence of 0, 0.1, 1 or 10 mM dCF for 2 days. After the initial drug exposure step, the cells were washed 3 times in drug free medium and resuspended in dAd at various concentrations of dAd (0, 10, 50 or 250 mM). The cells were then incubated in dAd for 2 more days. At the end of the treatment period, the cells were harvested for analysis by a TUNEL assay, a flow-cytometry-based method to quantify the percentage of cells that have undergone apoptosis. The level of apoptosis is expressed as “% FITC shift”. As a positive control, the cells were exposed to 5 mM camptothecin for 2 days. The results are presented below.

Results

The results are summarized below in Tables 1 and 2.

TABLE 1
Jurkat Cells

| | Drug After dCF | | |
|---|---|---|---|
| Conc dCF (uM) | Wash-Out | Conc dAd (uM) | % FITC Shift |
| 0 | none | — | 1.8 |
| | dAd | 10 | 1.6 |
| | | 50 | 4.2 |
| | | 250 | 22.2 |
| | Camptothecin | 5 | 80.2 |
| 0.1 | none | — | 1.1 |
| | dAd | 10 | 2.9 |
| | | 50 | 2.0 |
| | | 250 | 19.3 |
| 1 | none | — | 3.0 |
| | dAd | 10 | 1.6 |
| | | 50 | 7.1 |
| | | 250 | 69.6 |
| 10 | none | — | 1.0 |
| | dAd | 10 | 2.2 |
| | | 50 | 14.6 |
| | | 250 | 73.8 |

TABLE 2
U937 Cells

| | Drug After dCF | | |
|---|---|---|---|
| Conc dCF (uM) | Wash-Out | Conc (uM) | % FITC Shift |
| 0 | none | — | 1.2 |
| | dAd | 10 | 1.8 |
| | | 50 | 2.6 |
| | | 250 | 92.0 |
| | Camptothecin | 5 | 93.4 |
| 0.1 | none | — | 4.3 |
| | dAd | 10 | 2.0 |
| | | 50 | 90.8 |
| | | 250 | 77.4 |
| 1 | none | — | 2.0 |
| | dAd | 10 | 0.4 |
| | | 50 | 80.4 |
| | | 250 | 81.3 |
| 10 | none | — | 6.1 |
| | dAd | 10 | 71.8 |
| | | 50 | 71.9 |
| | | 250 | 90.6 |

Discussion of Results

Note that significant levels of apoptosis are achieved in Jurkat T-cells exposed to:
dCF at 0 uM for 2 days, followed by dAd at 250 uM for 2 days (22.2%)
dCF at 0.1 uM for 2 days, followed by dAd at 250 uM for 2 days (19.3%)
dCF at 1.0 uM for 2 days, followed by dAd at 50 uM for 2 days (7.1%)
dCF at 1.0 uM for 2 days, followed by dAd at 250 uM for 2 days (69.6%)
dCF at 10 uM for 2 days, followed by dAd at 50 uM for 2 days (14.6%)
dCF at 10 uM for 2 days, followed by dAd at 250 uM for 2 days (73.8%)
Camptothecin (80.2%)
   Note that significant levels of apoptosis are achieved in U937 cells exposed to:
dCF at 0 uM for 2 days, followed by dAd at 250 uM for 2 days (92%)
dCF at 0.1 uM for 2 days, followed by dAd at 50 uM for 2 days (90.8%)
dCF at 0.1 uM for 2 days, followed by dAd at 250 uM for 2 days (77.4%)
dCF at 1.0 uM for 2 days, followed by dAd at 50 uM for 2 days (80.4%)
dCF at 1.0 uM for 2 days, followed by dAd at 250 uM for 2 days (81.3%)
dCF at 10 uM for 2 days, followed by dAd at 10 uM for 2 days (71.8%)
dCF at 10 uM for 2 days, followed by dAd at 50 uM for 2 days (71.9%)
dCF at 10 uM for 2 days, followed by dAd at 250 uM for 2 days (90.6%)
Camptothecin (93.4%)

The results indicate that sequential treatment with dCF for two days, followed by dAd for two days, induces significant levels of apoptosis in both T-cells (Jurkat) and monocytoid cells (U937). This is the first demonstration that sequential exposure to dCF, followed by dAd, causes apoptosis of T-cells or monocytoid cells.

Significantly, 50 uM dAd induces 7.1% apoptosis in T-cells that are pre-treated with 0.1 uM dCF. By comparison, 50 uM dAd induces 90.8% apoptosis in U937 cells that are pre-treated with 0.1 uM dCF. Thus, monocytoid cells appear to be more sensitive than T-cells to the apoptotic effects of dCF/dAd.

Surprisingly, in the above mentioned experiment is that dAd appears to have pro-apoptotic activity in the absence of dCF. Thus, 92% of U937 cells, and 22.2% of Jurkat cells, became apoptotic in the presence of 250 uM dAd. Experiments are in progress to determine the minimum concentration of dAd that can induce apoptosis in T-cells and monocytoid cells. From about 75 micromolar to about 100 micromolar deoxyadenosine (dAdo) induces apoptosis in 4937 cells. The effect of duration on the pro-apoptotic effects of dAd will also be determined. For example, dAd at levels lower than 250 uM may continue to induce significant apoptosis in cell exposed beyond 2 days. It is also possible that 250 uM dAd may induce greater levels of apoptosis if the incubations are continued beyond 2 days.

Based upon the data summarized above, it is proposed that dAd alone may have the ability to induce apoptosis of T-cells, monocytes, and cells derived from monocytes, such as dendritic cells, inflammatory dendritic epithelial cells (IDEC), Langerhans cells, macrophages, and other types of antigen-presenting cells. The findings are of particular importance for the present invention, because the cellular targets of dCF and/or dAd include: T-cells, monocytes, and various cells derived from monocytes, such as dendritic cells, Langerhans cells and macrophages in the epidermis and dermis. Krueger, J. G. 2002, J. Amer. Acad. Dermatology, 46:1-23; Chairman and Krueger, J. G. 2004, Current Opinion in Rheumatology 16: 331-337.

### Example 22

Sequential and Simultaneous Exposure of U937 Cells to Deoxycoformycin (dCF) and Deoxyadenosine (dAd)

Introduction

Deoxycoformycin (dCF) is known to induce apoptosis in T-cells and monocytoid cells through the inhibition of adenosine deaminase (ADA). The concurrent exposure of the cells to dCF and deoxyadenosine (dAd) leads to greater levels of apoptosis than dCF alone; and the addition of dAd reduces the effective dose of dCF required to induce apoptosis (Niitsu et al. 1999; Niitsu et al. 2000; Bagnara, et al. 1992; for the latter reference see below). In other words, dAd potentiates the pro-apoptotic effect of dCF on T-cells and monocytoid cells.

A sequential exposure experiment has not been conducted before. Note that Bagnara et al. (1992) added dCF and then added dAd to human T-lymphoblasts. The interval between addition of dCF and dAd was 30 minutes. These investigators did not wash out the dCF before adding the dAd. Therefore, the cells were exposed to dCF alone and subsequently to both agents. Thus, this is a concurrent, not a sequential, exposure experiment.

Deoxycoformycin (dCF) binds extremely avidly to ADA with a dissociation half-life of 68 hours. Thus, it is hypothesized that the sequential exposure of the cells to dCF, followed by deoxyadenosine (dAd) will lead to greater levels of apoptosis than dCF alone. Whether cells in which ADA is inhibited by dCF are susceptible to the induction of apoptosis by dAd (after dCF has been washed out from the medium) was investigated. To test this hypothesis, cells were exposed to dCF for 2 days, washed the cells to remove free dCF from the cell culture medium, and then incubated the cells for another two days. The concentration of dAd needed to induce apoptosis in T cells (Jurkat) and monocytoid (U937) cells that have been exposed to dCF was determined.

Methods Simultaneous Exposure—U937 monocytoid cells were cultured in the presence of 0, 1, 2.5, 10, 20, 50, 100, 150, 200, and 250 μM dAdo for 2 days. Additionally, U937 monocytoid cells were cultured in the presence of 0.1, 1, and 10 μM dCF with the addition of dAdo in the concentration range of 1-50 μM for 2 days. At the end of the treatment period, the cells were harvested for analysis by a TUNEL assay, a flow-cytometry-based method to quantify the percentage of cells that have undergone apoptosis. The level of apoptosis is expressed as “% FITC shift”. As a positive control, the cells were exposed to 5 mM camptothecin for 2 days. The results are presented below.

Sequential Exposure—U937 monocytoid cells were cultured in the presence of 0 and 10 μM dCF for 2 days and then the cells were washed 3 times in drug free medium. At 0, 6, 24, and 48 hours, 50 μM dAd was added and the cells were incubated for an additional 2 days. At the end of the treatment period, the cells were harvested for analysis by a TUNEL assay, a flow-cytometry-based method to quantify the percentage of cells that have undergone apoptosis. The level of apoptosis is expressed as “% FITC shift”. As a positive control, the cells were exposed to 5 mM camptothecin for 2 days. The results are presented below.

Results

The results are summarized below in Tables 1 and 2.

TABLE 1
% Apoptotic U937 Cells at Different Concentrations
of dCF and dAd in Simultaneous Exposure

| Conc dCF (μM) | Conc dAd (μM) | % FITC Shift | % PI Shift |
|---|---|---|---|
| 0 | — | 1.5 | 1.4 |
| | 1 | 1.2 | 1.1 |
| | 2.5 | 2.1 | 1.0 |
| | 10 | 1.8 | 1.5 |
| | 20 | 1.7 | 1.8 |
| | 50 | 1.5 | 3.8 |
| | 100 | 33.7 | 15.9 |
| | 150 | 78.3 | 17.3 |
| | 200 | 62.6 | 19.7 |
| | 250 | 66.4 | 17.8 |
| | camptothecin | 79.5 | 43.4 |
| 0.1 | — | 3.1 | 1.4 |
| | 1 | 2.1 | 1.4 |
| | 2.5 | 7.4 | 11.5 |
| | 10 | 75.5 | 16.5 |
| | 20 | 82.6 | 19.2 |
| | 50 | 77.3 | 37.9 |
| 1 | — | 2.4 | 1.7 |
| | 1 | 1.8 | 4.7 |
| | 2.5 | 61.4 | 28.4 |
| | 10 | 70.0 | 28.3 |
| | 20 | 77.8 | 42.8 |
| | 50 | 59.8 | 60.5 |
| 10 | — | 4.3 | 1.2 |
| | 1 | 6.8 | 15.7 |
| | 2.5 | 78.6 | 21.9 |
| | 10 | 74.4 | 30.6 |
| | 20 | 65.8 | 46.3 |
| | 50 | 67.7 | 58.8 |

TABLE 2
% Apoptotic U937 Cells at Different Times After Wash-out of dCF

| Time After Wash-Out | | | |
|---|---|---|---|
| (hr) | Conc dCF (μM) | % FITC Shift | % PI Shift |
| 0 | 0 | 1.6 | 1.4 |
| 0 | 10 | 76.0 | 28.6 |
| 0 | 0 + camptothecin | 84.3 | 29.2 |
| 6 | 0 | 2.2 | 1.3 |
| 6 | 10 | 66.3 | 18.6 |
| 24 | 0 | 2.2 | 1.1 |
| 24 | 10 | 1.7 | 7.9 |
| 48 | 0 | 1.0 | 2.0 |
| 48 | 10 | 1.4 | 3.3 |

Discussion

Deoxyadenosine, at concentrations in the micromolar range and in the absence of dCF, induces apoptosis in monocytoid cells (e.g., dAd alone is toxic at all concentrations >100 μM). The combination of dAdo (2.5 μM) and dCF (1 μM) produces and even greater effect. The simultaneous exposure is effective at lower drug concentrations than the wash-out. In a previous study, done by wash-out, 10 μM dAd was not effective at 0.1 or 1 μM dCF, although it was effective at 10 μM dCF. In the simultaneous study, an effect of 2.5 μM dAd at dCF concentrations as low as 0.1 μM was demonstrated and even an effect of 1 μM dAd at 10 μM dCF was demonstrated. Thus, while simultaneous exposure requires less drug than wash-out, wash-out is still an effective approach. In fact, the wash-out experiments demonstrate that the effect is still present when dAd is added 6 hrs after wash-out (Table 2). There may even be an effect after 24 hrs (Table 2).

The results suggest that the combination may have a better therapeutic index compared to dCF alone, and that dAd alone is a viable treatment alternative, especially since the target cells reside in the epidermis: 1) langerhans cells, which are derived from monocytes; and 2) CD8+T-cells, including CD45RO+ memory cells, to which the causal antigen is presented by the langerhans cells.

Thus, the topical use of combination therapy or dAd alone can eliminate the two primary cell types that drive inflammation in psoriatic plaque. This is different than other treatments, including corticosteroids, retinoids, vitamin D analogues, and calcineurin inhibitors, in that none of them can selectively eliminate langerhans cells and T-cells.

### Example 23

Step 1. Apply a topical formulation that contains dAdo in the concentration range of about 0.00005 to about 5.0 w/w % with or without an occlusive dressing. The number of treatments with dAdo could be about 1 to about 14 days (e.g., about 1 to about 7 days). After each administration the inflamed area may be washed with soap and water and dried before next application. Treatment area is up to 100% of BSA. At this point ADA is inhibited in the treated cells so dAdo will not be deaminated in those cells. This will allow dAdo to preferentially be converted to dATP generating the elevated levels of dATP necessary to induce apoptosis.

Step 2. After Step 1 is completed, over the next 0-5 days no treatment will occur. This time will be used to monitor the affect of the treatment and give time for the system to equilibrate before the start of the next sequential treatment.

Step 3. If the desired response is not obtained, Step 1 will be repeated for a period of up to 12-weeks.

### Example 24

Exposure of U937 Monocytoid Cells to Deoxyadenosine (dAd), Adenosine (Ad) and Adenine (A)

Introduction

Deoxycoformycin (dCF) is known to induce apoptosis in T-cells and monocytoid cells through the inhibition of adenosine deaminase (ADA). The concurrent exposure of the cells to dCF and deoxyadenosine (dAd) leads to greater levels of apoptosis than dCF alone. Furthermore, the addition of dAd reduces the effective dose of dCF required to induce apoptosis (Niitsu et al; 1999; Niitsu et al 2000; Bagnara, et al 1992). In other words, dAd is believed to potentiate the pro-apoptotic effect of dCF on T-cells and monocytoid cells.

Materials and Methods

U937 monocytoid cells were cultured in the presence of dAd at various concentrations of dAd (0, 50, 75, 100, 250 and 1000 micromoles/liter). The cells were incubated in dAd for 2 days. At the end of the treatment period, the cells were harvested for analysis by a TUNEL assay, a flow-cytometry-based method to quantify the percentage of cells that have undergone apoptosis. The level of apoptosis expressed as “% FITC shift”. The level of cell cycle arrest is expressed as “% PI shift”. As a positive control, the cells were exposed to 5 mM camptothecin for 2 days.

Results and Discussion

Surprisingly, initial experiments demonstrated that dAd (without the addition of dCF) induces apoptosis in U937 cells. A dose ranging experiment was conducted to determined the minimum concentration of dAd needed to induce apoptosis in monocytoid (U937) cells which have not been exposed to dCF. The results are summarized in Table 1 below (U937 cells).

| Drug | Concentration (uM) | % FITC Shift | % PI Shift |
|---|---|---|---|
| dAd (Sigma) | 50 | 1.5 | 3.2 |
| | 75 | 1.8 | 8.8 |
| | 100 | 9.0 | 12.1 |
| | 250 | 65.8 | 12.0 |
| | 1000 | 79.2 | 29.5 |
| dAd (Acros) | 50 | 0.7 | 2.9 |
| | 75 | 1.8 | 7.1 |
| | 100 | 3.5 | 9.8 |
| | 250 | 54.1 | 14.6 |
| | 1000 | 80.2 | 32.6 |
| Adenosine | 50 | 0.7 | 1.7 |
| | 75 | 0.6 | 2.1 |
| | 100 | 0.6 | 2.0 |
| | 250 | 0.6 | 1.9 |
| | 1000 | 2.9 | 7.9 |
| Adenine | 50 | 0.4 | 2.0 |
| | 75 | 0.6 | 2.4 |
| | 100 | 0.3 | 2.5 |
| | 250 | 0.7 | 3.4 |
| | 1000 | 2.9 | 9.5 |
| No drug | | 1.0 | 2.1 |
| Camptothecin | 5 | 72.8 | 32.2 |

The data indicate that deoxyadenosine (dAd) induces apoptosis at 100 uM and progressively more at 250 and 1000. dAd arrests cell cycle beginning at 75 uM.

Adenosine and adenine show little or no effect on apoptosis or cell cycle up to 250 uM and a slight hint of an effect at 1000 uM.

### Example 25

2-deoxyadenosine (dAdo)

1) 2-deoxyadenosine (dAdo) in a Topical Formulation Including Penetration Enhancer(s) Used to Treat Topical Skin Disorders

dAdo can be toxic to monocytes and lymphocytes at concentrations greater than 100 μM (Example 21 and 23, Siaw et al, Rosowsky et al). The necessity for these high levels is due to the rapid deamination of dAdo by ADA. Due to this, a topical formulation of dAdo should include a penetration enhancer, which is “an ingredient of a topical formulation that causes increased penetration into the skin.” Additionally, a penetration enhancer could be any technique used to increase the permeation rate of dAdo, e.g. a solution of bath water (5-130° C.) containing dAdo in the concentration range of 0.00005%-5%. A penetration enhancer is necessary because of the high concentrations of dAdo needed in situ.

To date dAdo has never been formulated with penetration enhancer(s) for a topical product. With aggressive formulations with penetration enhancers, it is conceivable that penetration rates could be as high as 50% absorption, but more likely in the range of 5-15%. The concentration range for dAdo is about 0.00005 to about 5 w/w %. The penetration enhancers include, but are not limited to, DMSO, EDTA, EGTA, Loramide DEA, Ethoxydiglycol, NMP, Triacetin, Propylene Glycol, Benzyl Alcohol, Sodium Laureth, Sulfate, Dimethyl Isosorbide, Isopropyl Myristate, Isopropyl Palmitate, Isopropyl Isostearate, Propylene Glycol Monostearate, Diisopropyl Adipate, Diethyl Sebacate, Oleic Acid, Ethyl Oleate, Glyceryl Oleate, Caprylic/Capric Triglyceride, Propylene Glycol Dicaprylate/Dicaprate, Laureth-4, Oleth-2, Oleth-20, Propylene Carbonate, Nonoxynol-9, Nonoxynol-15, and a solution (warn or cold) of dAdo for immersing inflamed skin.

2) Concentration Ranges and Treatment Schedules when dAdo is Used as a Monotherapy

a) Apply a topical formulation of dAdo including of a penetration enhancer in the concentration range of about 0.00005 to about 5 w/w % to the inflamed areas, with or without an adhesive skin patch, at 100% of the BSA, for a period of 1 day to 6 months. The number of concurrent treatments could be up to as many as 3 treatments per day for a period of up to about 12 weeks. After each treatment the inflamed area may be washed with soap and water and dried before the next application.

b) Apply a topical formulation of dAdo including of a penetration enhancer in the concentration range of about 0.00005 to about 5 w/w % to the inflamed areas, with or without an adhesive skin patch, at 30% of the BSA, for a period of 1 day to 6 months. The number of concurrent treatments could be up to as 3 treatments per day for a period of up to about 12 weeks. After each treatment the inflamed area may be washed with soap and water and dried before the next application.

c) Apply a topical formulation of dAdo including of a penetration enhancer in the concentration range of about 0.00005 to about 5 w/w % to the inflamed areas, with or without an adhesive skin patch, at 10% of the BSA, for period of 1 day to about 6 months. The number of concurrent treatments could be up to as many as 3 treatments per day for a period of up to about 12 weeks. After each treatment the inflamed area may be washed with soap and water and dried before the next application.

The dAdo concentrations proposed above in a)-c) are in the concentration range to inhibit the growth of lymphocytes in vitro. From the above examples, at the most severe case where 40 grams of a 5% dAdo topical formulation is delivered per day this treatment is believed to be extremely safe. No safety data is present at the current time for dAdo, but this compound is thought to be benign because of its ability to be readily catabolized to 2′-deoxyinosine, so in the extreme case of a 5% dAdo formulation the toxicity should be minimal.

Once toxicity data is generated, prolonging the treatment schedule, reducing concentrations of dAdo, and/or decreasing the penetration rate of the topical formulations would increase the safety margins and the therapeutic index if necessary. Based upon the in vitro data and formulations it is concluded that dAdo may be used as a safe topical treatment for mild, moderate, and severe autoimmune skin diseases.

3) Topical Formulation for 2-deoxyadenosine (dAdo)

One-Part Formulation—dAdo, like other nucleosides, are often stable under specific conditions. Prophetic formulations are given as examples. A one-part formulation includes dAdo combined with a base topical delivery system, such as a solution, gel, cream, or ointment. The concentration of water varies with each formulation where a solution base may have 25-80 w/w % water; a gel base may have 25-95 w/w % water; a cream base may have 50-80 w/w % water; and an ointment base may have between 1-10 w/w % water. Nucleosides and their analogs are most stable in a pH range of 5 to 9, so all formulations are buffered respectively. Below are four-examples of a one-part formulation using different bases.

| | | |
|---|---|---|
| Component | Function | % w/w |

a) Solution Formulation

| | | |
|---|---|---|
| 2-deoxyadenosine | Active | 0.00005-5 |
| Isopropyl Alcohol | Solvent | 35-55 |
| Propylene Glycol | Penetration | 1-15 |
| | Enhancer | |
| Isopropyl Palmitate | Penetration | 1-10 |
| | Enhancer | |
| Hydroxypropyl Cellulose | Thickening | 0-5 |
| | agent | |
| Phosphoric Acid | Acidifying | pH 5-9 |
| | Agent | |
| Dibasic sodium phosphate | Base | 0.01-1.5 |
| Menthol | Odorant | 0-1 |
| Purified Water | Diluent | 25-80 |

b) Gel Formulation

| | | |
|---|---|---|
| 2-deoxyadenosine | Active | 0.00005-5 |
| Propylene glycol | Penetration | 0.1-10 |
| | Enhancer | |
| Isopropyl Palmitate | Penetration | 1-10 |
| | Enhancer | |
| Methylparaben | Preservative | 0.01-0.1 |
| Propylparaben | Preservative | 0.01-0.1 |
| Edetate Disodium | Chelating agent | 0.01-0.1 |
| Dibasic sodium phosphate | Basic Agent | 0.01-1.5 |
| Carbomer | Gelling Agent | 0.1-2 |
| Phosphoric Acid | Neutralizing | pH 5-9 |
| | Agent | |
| Ethanol | Solvent | 0-75 |
| Purified Water | Solvent | 25-95 |

c) Cream Formulation

| | | |
|---|---|---|
| 2-deoxyadenosine | Active | 0.00005-5 |
| Sorbitol 70% Solution | Humectant | 1-3 |
| Emulsifying Wax | Cream Base | 5-25 |
| Glycerin | Emollient | 0-20 |
| Isopropyl Palmitate | Penetration | 1-10 |
| | Enhancer | |
| Benzyl alcohol | Preservative | 0.1-0.5 |
| Edetate disodium | Chelating agent | 0.01-0.55 |
| Dibasic sodium phosphate | Basic Agent | 0.01-0.55 |
| Phosphoric Acid | Neutralizing Agent | pH 5-9 |
| Ceteth 20 | Surfactant | 0.5-5 |
| Mineral Oil | Emollient | 0-55 |
| Purified Water | Solvent | 50-80 |

d) Ointment Formulation

| Excipient | Function | % w/w |
|---|---|---|
| 2-deoxyadenosine | Active | 0.00005-5 |
| Microcrystalline Wax | Ointment base | 0-15 |
| White Petrolatum | Ointment base | 55-99 |
| Tocopherol | Anti-oxidant | 0-0.5 |
| Steareth-2 | Surfactant | 1-10 |
| Propylene Glycol | Penetration | 1-10 |
| | Enhancer | |
| Isopropyl Palmitate | Penetration | 1-10 |
| | Enhancer | |
| Edetate disodium | Chelating agent | 0.001-0.55 |
| Dibasic sodium phosphate | Basic Agent | 0.01-0.55 |
| Phosphoric Acid | Neutralizing Agent | pH 5-9 |
| Purified Water | Solvent | 0-10 |

Two-Part Formulation—A two-part formulation would consist of a dAdo alone or lyophilized in Container A (e.g., a syringe) and a topical vehicle in Container B (e.g., a syringe). Prior to administration, dAdo and delivery components are mixed thoroughly, for example, by repetitively transferring the components between syringes. In this way, the product is reconstituted.

A two-part (mix and use) formulation minimizes the degradation that may be possible due to excipient or vehicle interaction since dAdo is exposed to the excipients only briefly before application. The preferred two-part formulation uses a cream base; however, a gel, solution, or ointment base may also be implemented if necessary. An example of a two-part cream formulation is listed below.

e) Two-Part Cream Formulation
Part 1 - Drug solution for lyophilization: Container/Syringe A

| Component | Function | % w/v |
|---|---|---|
| 2-deoxyadenosine | Active | 0.00013-13 |
| Mannitol | Bulking Agent | 0.0-8.0 |
| Polyethylene Glycol 4000 | Bulking Agent | 0.0-8.0 |
| Water for Injection | Solvent | QS 100 |

Fill Target = 0.4 mL in a 3-cc Becton Dickinson Sterifill Syringe
Final Drug Amount = 0.05 mg

Part 2 - Cream Base for reconstitution: Container/Syringe B

| Component | Function | % w/w |
|---|---|---|
| Sorbitol 70% Solution | Humectant | 1.0-5.0 |
| Emulsifying Wax | Cream Base | 5.0-20.0 |
| Glycerin | Emollient | 0-5.0 |
| Isopropyl Palmitate | Penetration | 2.0-10.0 |
| Benzyl alcohol | Preservative | 0.1-0.5 |
| Dibasic sodium phosphate | Basic Agent | 0.1-0.5 |
| Phosphoric Acid | Neutralizing Agent | pH 5-9 |
| Purified Water | Solvent | QS 100 |

Fill Target = 1 g in a 3-cc female Ultratek Syringe

Final Reconstituted Product

| Component | Function | % w/w |
|---|---|---|
| 2-deoxyadenosine | Active | 0.00005-5 |
| Mannitol | Bulking Agent | 0-21 |
| Polyethylene Glycol 4000 | Bulking Agent | 0-21 |
| Sorbitol 70% Solution | Humectant | 1-5 |
| Emulsifying Wax | Cream Base | 5-20 |
| Glycerin | Emollient | 0-5 |
| Isopropyl Palmitate | Penetration | 2-10 |
| Benzyl alcohol | Preservative | 0.1-0.5 |
| Dibasic sodium phosphate | Basic Agent | 0.01-0.10 |
| Phosphoric Acid | Neutralizing Agent | pH 5-9 |
| Purified Water | Solvent | QS 100 |

Reconstitution: Syringe A and Syringe B are coupled together by means
of integrated Leur Loks.
The product is mixed by first passing the Cream Base into the Drug
Syringe, and then returning the mixture to Syringe B. The product is
mixed back and forth in this manner 30-100 times to completely mix
the drug with the cream. The cream formulation is then dispensed from
the Syringe A for application.

4) Concentration Ranges and Treatment Schedules when dAdo Treatment Includes a Corticosteroid in Combination

a) Apply a topical formulation of dAdo in the concentration range of about 0.00005 to about 5 w/w % and a corticosteroid in the concentration range of 0.00005-1 w/w % to the inflamed areas, with or without an adhesive skin patch, at 100% of the BSA, for a period of 1 day to 6 months. The number of concurrent treatments could be up to as many as 3 treatments per day for a period of up to about 12 weeks. After each treatment the inflamed area may be washed with soap and water and dried before the next application.

b) Apply a topical formulation of dAdo in the concentration range of about 0.00005 to about 5 w/w % and a corticosteroid in the concentration range of 0.00005-1 w/w % to the inflamed areas, with or without an adhesive skin patch, at 30% of the BSA, for a period of 1 day to 6 months. The number of concurrent treatments could be up to as many as 3 treatments per day for a period of up to about 12 weeks. After each treatment the inflamed area may be washed with soap and water and dried before the next application.

c) Apply a topical formulation of dAdo in the concentration range of about 0.00005 to about 5 w/w % and a corticosteroid in the concentration range of 0.00005-1 w/w % to the inflamed areas, with or without an adhesive skin patch, at 10% of the BSA, for a period of 1 day to 6 months. The number of concurrent treatments could be up to as many as 3 treatments per day for a period of up to about 12 weeks. After each treatment the inflamed area may be washed with soap and water and dried before the next application.

d) Apply a topical formulation of dAdo in the concentration range of about 0.00005 to about 5 w/w % and calcipotriene in the concentration range of 0.00005-1 w/w % to the inflamed areas, with or without an adhesive skin patch, at 100% of the BSA, for a period of 1 day to 6 months. The number of concurrent treatments could be up to as many as 3 treatments per day for a period of up to about 12 weeks. After each treatment the inflamed area may be washed with soap and water and dried before the next application.

e) Apply a topical formulation of dAdo in the concentration range of about 0.00005 to about 5 w/w % and calcipotrien in the concentration range of 0.00005-1 w/w % to the inflamed areas, with or without an adhesive skin patch, at 30% of the BSA, for a period of 1 day to 6 months. The number of concurrent treatments could be up to as 3 treatments per day for a period of up to about 12 weeks. After each treatment the inflamed area may be washed with soap and water and dried before the next application.

f) Apply a topical formulation of dAdo in the concentration range of about 0.00005 to about 5 w/w % and calcipotriene in the concentration range of 0.00005-1 w/w % to the inflamed areas, with or without an adhesive skin patch, at 10% of the BSA, for a period of 1 day to 6 months. The number of concurrent treatments could be up to as many as 3 treatments per day for a period of up to about 12 weeks. After each treatment the inflamed area may be washed with soap and water and dried before the next application.

### Example 26

2-chlorodeoxyadenosine (Cladrabine or CdA)

1) Range of 2-chlorodeoxyadenosine (CdA) Concentrations in Topical Formulations

To date, CdA has never been formulated into a topical product. Several potential solutions to the instability issue discussed above are provided in the below examples. Experiments conducted in vitro have generally concluded that CdA is effective at cell killing by apoptosis in 2-4 days. The degree of penetration of each drug into the dermis is not known, thus, formulations are being developed using a combination of empirical and inductive experiments. The concentration range for CdA is guided by safety data from clinical studies in which leukemia patients received IV CdA. Based upon in vitro and in vivo data, CdA monotherapy would be effective, provided that endogenous (intradermal) CdA accumulates during therapy. However, the effect may take many days to gain momentum.

The oncology dose is 3.6 mg/m² every day for 7 days. Based upon the assumption that the average body surface area is 1.8 m², a safe dose of CdA is 6.48 mg every day. For oncology patients, at this dosage, the adverse events were mild to moderate and diminished with treatment, therefore 3.6 mg/m²/day is referred to as the safe dose that can be administered systemically. At this dose range, patients with hairy cell leukemia (HCL) achieve responses and complete remissions, within 1 week of treatment. (Morris et al., 1997)

Based upon topical formulations that are similar to the formulations of CdA, it is estimated that the systemic absorption of CdA will be no more than 7%. Calculations of safety margins have been performed, based upon this assumption and the known toxicity profile of CdA when administered IV for oncology indications. Systemic absorption is a concern, because of the potential for serious adverse events, such as lymphopenia and renal toxicity. At oncology doses, these adverse reactions are usually minimized, but the therapeutic index is narrow. Another factor that plays a role in the safety of the formulation is the amount of product administered each day. Approximately 4 grams, 12 grams, and 40 grams of a topical formulation are estimated to cover 10%, 30%, and 100% of the body surface area. Below are some examples of safety margin calculations using CdA monotherapy, based upon 7% estimated fractional absorption, and various dose intensities.

2) Concentration Ranges, Treatment Schedules, and Safety Margins when CdA is Used as a Monotherapy

a) Apply a topical formulation of CdA in the concentration range of about 0.000001 to about 0.1 w/w % to the inflamed areas, with or without an adhesive skinpatch, at 100% of the BSA, for a period of 1 day to 6 months. The number of concurrent treatments could be up to as many as 3 treatments per day for a period of up to about 12 weeks. After each treatment the inflamed area may be washed with soap and water and dried before the next application.

Using the highest concentration of 0.1% (1.0 mg/g), 100% BSA coverage (40 grams) and 7% systemic absorption, patients would be systemically exposed to 2.8 mg of CdA per day. This corresponds to a safety margin of 2.3 or 2.3 times more safe than the oncology treatment (6.48 mg/day). For the subsequent lower concentrations the safety factor would be 23, 230, 2300, 23000, and 230000.

b) Apply a topical formulation of CdA in the concentration range of about 0.000001 to about 0.1 w/w % to the inflamed areas, with or without an adhesive skin patch, at 30% of the BSA, for a period of 1 day to 6 months. The number of concurrent treatments could be up to as many as 3 treatments per day for a period of up to about 12 weeks. After each treatment the inflamed area may be washed with soap and water and dried before the next application.

Using the highest concentration of 0.1% (1.0 mg/g), 30% BSA coverage (12 grams) and 7% systemic absorption, patients would be systemically exposed to 0.84 mg of CdA per day. This corresponds to a safety margin of 7.7. For the subsequent lower concentrations, the safety factor would be 77, 770, 7700, 77000 and 770000.

c) Apply a topical formulation of CdA in the concentration range of about 0.000001 to about 0.1 w/w % to the inflamed areas, with or without an adhesive skin patch, at 10% of the BSA, for period of 1 day to about 6 months. The number of concurrent treatments could be up to as many as 3 treatments per day for a period of up to about 12 weeks. After each treatment the inflamed area may be washed with soap and water and dried before the next application.

Using the highest concentration of 0.1% (1.0 mg/g), 10% BSA coverage (4 grams) and 7% systemic absorption, patients would be systemically exposed to 0.28 mg of CdA per day. This corresponds to a safety margin of 23. For the subsequent lower concentrations, the safety factor would be 230, 2300, 23000, 230000, and 2300000.

The CdA concentrations described above in a)-c) are the concentration ranges to inhibit the growth of lymphocytes in vitro. From the above examples, at the most sever case where 40 grams of a 0.1% CdA topical formulation is delivered per day this treatment would still be 2.3 times safer than the oncology dose. For most patients with mild to moderate topical autoimmune conditions, the application would be less than 12 grams per day. At 12 grams per day this treatment would be 7.7 times safer than the oncology dose. To improve the therapeutic index, the treatment schedule could be extended and lower concentrations administered thereby increasing the safety margins in the range of 7.7 to 2300000.

Another topical formulation would be 0.00001% CdA (0.0001 mg/g), which is equivalent to approximately 25 nM achieved in the skin assuming 7% penetration. According to in vitro data, 25 nM is needed to cause complete inhibition of monocytes. (Niitsu et al, 2000) Using the most dose intensive regimen of 40 grams delivered each day, a topical formulation consisting of 0.00001% CdA and 7% penetration would be considered 23000 times safer than the oncology dose.

To further improve the therapeutic index, the treatment schedule could be prolonged while using reduced concentrations of CdA thereby increasing the safety margins. Based upon the calculations listed above, and the ability to stabilize the molecule by minimizing excipient interaction, it is concluded that CdA may be used as a safe topical treatment for mild, moderate, and severe autoinmune skin diseases.

3) Topical Formulation for 2-chlorodeoxyadenosine (CdA or Cladrabine)

One-Part Formulation

CdA, like other nucleosides and their analogs, are often stable under specific conditions. Prophetic formulations are given below as examples. A one-part formulation includes CdA combined with a base topical delivery system, such as a solution, gel, cream, or ointment. The concentration of water varies with each formulation where a solution base may have 25-80 w/w % water; a gel base may have 25-95 w/w % water; a cream base may have 50-80 w/w % water; and an ointment base may have between 1-10 w/w % water. Nucleosides and their analogs are most stable in a pH range of 5 to 9, so all formulations are buffered respectively. Below are four-examples of a one-part formulation using different bases.

| | | |
|---|---|---|
| Component | Function | % w/w |

a) Solution Formulation

| | | |
|---|---|---|
| 2-chlorodeoxyadenosine | Active | 0.000001-0.1 |
| Isopropyl Alcohol | Solvent | 35-55 |
| Propylene Glycol | Solvent | 1-15 |
| Hydroxypropyl Cellulose | Thickening | 0-5 |
| | agent | |
| Phosphoric Acid | Acidifying | pH 5-9 |
| | Agent | |
| Dibasic sodium phosphate | Base | 0.01-1.5 |
| Menthol | Odorant | 0-1 |
| Purified Water | Diluent | 25-80 |

b) Gel Formulation

| | | |
|---|---|---|
| 2-chlorodeoxyadenosine | Active | 0.000001-0.1 |
| Propylene glycol | Solvent | 0.1-10 |
| Methylparaben | Preservative | 0.01-0.1 |
| Propylparaben | Preservative | 0.01-0.1 |
| Edetate Disodium | Chelating agent | 0.01-0.1 |
| Dibasic sodium phosphate | Basic Agent | 0.01-1.5 |
| Carbomer | Gelling Agent | 0.1-2 |
| Phosphoric Acid | Neutralizing | pH 5-9 |
| | Agent | |
| Ethanol | Solvent | 0-75 |
| Purified Water | Solvent | 25-95 |

c) Cream Formulation

| | | |
|---|---|---|
| 2-chlorodeoxyadenosine | Active | 0.000001-0.1 |
| Sorbitol 70% Solution | Humectant | 1-3 |
| Emulsifying Wax | Cream Base | 5-25 |
| Glycerin | Emollient | 0-20 |
| Isopropyl Palmitate | Penetration | 1-10 |
| Benzyl alcohol | Preservative | 0.1-0.5 |
| Edetate disodium | Chelating agent | 0.01-0.55 |
| Dibasic sodium phosphate | Basic Agent | 0.01-0.55 |
| Phosphoric Acid | Neutralizing Agent | pH 5-9 |
| Ceteth 20 | Surfactant | 0.5-5 |
| Mineral Oil | Emollient | 0-55 |
| Purified Water | Solvent | 50-80 |

d) Ointment Formulation

| Excipient | Function | % w/w |
|---|---|---|
| 2-chlorodeoxyadenosine | Active | 0.000001-0.1 |
| Microcrystalline Wax | Ointment base | 0-15 |
| White Petrolatum | Ointment base | 55-99 |
| Tocopherol | Anti-oxidant | 0-0.5 |
| Steareth-2 | Surfactant | 1-10 |
| Propylene Glycol | Solvent | 1-10 |
| Edetate disodium | Chelating agent | 0.001-0.55 |
| Dibasic sodium phosphate | Basic Agent | 0.01-0.55 |
| Phosphoric Acid | Neutralizing Agent | pH 5-9 |
| Purified Water | Solvent | 0-10 |

Two-Part Formulation—Leustatin®, a commercial oncology injectable product has a two-year shelf life and it is supplied as an isotonic solution of 2-chloroadenosine (CdA) and sodium chloride. The solution has a pH range from 5.5 to 8.0. Given the two-year solution stability of CdA at 2-8° C., a two-part formulation would consist of a CdA isotonic solution in a pH range of 5.5 to 8.0 in container A (such as a syringe) and a topical vehicle in container B (such as a syringe). Prior to administration, the CdA solution and delivery components are mixed thoroughly, such as by repetitively transferring the components between the syringes. In this way, the product is reconstituted.

A two-part (mix and use) formulation minimizes the degradation that may be possible due to excipient or vehicle interaction since CdA is exposed to the excipients only briefly before application. The preferred two-part formulation uses a cream base; however, a gel, solution, or ointment base may also be implemented if necessary. An example of a two-part cream formulation is listed below.

e) Two-Part Cream Formulation
Part 1 - Drug solution for lyophilization: Container/Syringe A

| Component | Function | % w/v |
|---|---|---|
| 2-chlorodeoxyadenosine | Active | 0.0000026-0.26 |
| Mannitol | Bulking Agent | 0.0-8.0 |
| Polyethylene Glycol 4000 | Bulking Agent | 0.0-8.0 |
| Water for Injection | Solvent | QS 100 |

Fill Target = 0.4 mL in a 3-cc Becton Dickinson Sterifill Syringe
Final Drug Amount = 0.05 mg

Part 2 - Cream Base for reconstitution: Container/Syringe B

| Component | Function | % w/w |
|---|---|---|
| Sorbitol 70% Solution | Humectant | 1.0-5.0 |
| Emulsifying Wax | Cream Base | 5.0-20.0 |
| Glycerin | Emollient | 0-5.0 |
| Isopropyl Palmitate | Penetration | 2.0-10.0 |
| Benzyl alcohol | Preservative | 0.1-0.5 |
| Dibasic sodium phosphate | Basic Agent | 0.1-0.5 |
| Phosphoric Acid | Neutralizing Agent | pH 5-9 |
| Purified Water | Solvent | QS 100 |

Fill Target = 1 g in a 3-cc female Ultratek Syringe

Final Reconstituted Product

| Component | Function | % w/w |
|---|---|---|
| 2-chlorodeoxyadenosine | Active | 0.000001-0.1 |
| Mannitol | Bulking Agent | 0-21 |
| Polyethylene Glycol 4000 | Bulking Agent | 0-21 |
| Sorbitol 70% Solution | Humectant | 1-5 |
| Emulsifying Wax | Cream Base | 5-20 |
| Glycerin | Emollient | 0-5 |
| Isopropyl Palmitate | Penetration | 2-10 |
| Benzyl alcohol | Preservative | 0.1-0.5 |
| Dibasic sodium phosphate | Basic Agent | 0.01-0.10 |
| Phosphoric Acid | Neutralizing Agent | pH 5-9 |
| Purified Water | Solvent | QS 100 |

Example of Reconstitution: Syringe A and Syringe B are coupled
together by means of integrated Leur Loks. The product is mixed by
first passing the Cream Base into the Drug Syringe, and then returning
the mixture to Syringe B. The product is mixed back and forth in this
manner 30-100 times to completely mix the drug with
the cream. The cream formulation is then dispensed from the Syringe A
for application.

### Example 27

2-fluoroadenine-9β-D-Arabinofuranoside (F-Ara-A)

1) Range of 2-fluoroadenine-9β-D-Arabinofuranoside (F-Ara-A) Concentrations in Topical Formulations

To date, the nucleoside F-Ara-A has not been formulated into a topical delivery system, although the corresponding nucleotide of F-Ara-A, fludarabine was used in an Aquaphor® ointment as the topical delivery base to evaluate the efficacy in psoriasis. (Nouri, et al, 1997) F-Ara-A differs from fludarabine in the fact that fludarabine has a highly charged 5′monophosphate moiety. The active metabolite for both is F-Ara-ATP. F-Ara-A in a unique formulation designed to carry the drug into the skin would have a greater benefit since non-charge molecules can penetrate cell membranes more readily and that a formulation designed specifically for penetration of F-Ara-A would ensure penetration.

Several potential solutions to the instability issues provided by F-Ara-A are provided as examples. Experiments conduced in vitro have generally concluded that F-Ara-A is effective at cell killing by apoptosis in 2-4 days. The degree of penetration of each drug into the dermis is unknown, so formulations are being developed using a combination of empirical and inductive experiments. The concentration range for F-Ara-A is guided by safety data from clinical studies in which leukemia patients received IV F-Ara-A. Based upon in vitro and in vivo data, F-Ara-A monotherapy would be effective, provided that endogenous (intradermal) F-Ara-A accumulates during therapy. However, the effect may take many days to gain momentum.

The oncology dose is 25 mg/m² every day for 5 days. Based upon the assumption that the average body surface area is 1.8 m², a safe dose of F-Ara-A is 45 mg every day. For oncology patients, at this dosage, the adverse events were mild to moderate and diminished with treatment, therefore 25 mg/m²/day is referred to as the safe dose that can be administered systemically. At this dose range, patients with chronic lymphocytic leukemia (CLL) achieve responses in the first few weeks, and complete remissions, on average, after 3 months of treatment. (Kolesar et al, 1996)

Based upon topical formulations that are similar to the formulations of F-Ara-A, it is estimated that the systemic absorption of F-Ara-A will be no more than 7%. Calculations of safety margins have been performed, based upon this assumption and the known toxicity profile of F-Ara-A when administered IV for oncology indications. Systemic absorption is a concern, because of the potential for serious adverse events, such as lymphopenia and renal toxicity. At oncology doses, these adverse reactions are usually minimized, but the therapeutic index is narrow. Another factor that plays a role in the safety of the formulation is the amount of product administered each day. Approximately 4 grams, 12 grams, and 40 grams of a topical formulation are estimated to cover 10%, 30%, and 100% of the body surface area. Below are some examples of safety margin calculations using F-Ara-A monotherapy, based upon 7% estimated fractional absorption, and various dose intensities.

2) Concentration Ranges, Treatment Schedules, and Safety Margins when 2-fluoroadenine-9β-D-Arabinofuranoside (F-Ara-A or Fludarabine des-phosphate) is Used as a Monotherapy

a) Apply a topical formulation of F-Ara-A in the concentration range of about 0.000001 to about 1.0 w/w % to the inflamed areas, with or without an adhesive skin patch, at 100% of the BSA, for a period of 1 day to 6 months. The number of concurrent treatments could be up to as many as 3 treatments per day for a period of up to about 12 weeks. After each treatment the inflamed area may be washed with soap and water and dried before the next application.

Using the highest concentration of 1.0% (10 mg/g), 100% BSA coverage (40 grams) and 7% systemic absorption, patients would be systemically exposed to 28 mg of F-Ara-A per day. This corresponds to a safety margin of 1.6 or 1.6 times more safe than the oncology treatment (45 mg/day). For the subsequent lower concentrations the safety factor would be 16, 160, 1600, 16000, 160000, and 1600000.

b) Apply a topical formulation of F-Ara-A in the concentration range of about 0.000001 to about 1.0 w/w % to the inflamed areas, with or without an adhesive skin patch, at 30% of the BSA, for a period of 1 day to 6 months. The number of concurrent treatments could be up to as 3 treatments per day for a period of up to about 12 weeks. After each treatment the inflamed area may be washed with soap and water and dried before the next application.

Using the highest concentration of 1.0% (10 mg/g), 30% BSA coverage (12 grams) and 7% systemic absorption, patients would be systemically exposed to 8.4 mg of F-Ara-A per day. This corresponds to a safety margin of 5.4. For the subsequent lower concentrations, the safety factor would be 54, 540, 5400, 54000, 540000, and 5400000.

c) Apply a topical formulation of F-Ara-A in the concentration range of about 0.000001 to about 1.0 w/w % to the inflamed areas, with or without an adhesive skin patch, at 10% of the BSA, for period of 1 day to about 6 months. The number of concurrent treatments could be up to as many as 3 treatments per day for a period of up to about 12 weeks. After each treatment the inflamed area may be washed with soap and water and dried before the next application.

Using the highest concentration of 1.0% (10 mg/g), 10% BSA coverage (4 grams) and 7% systemic absorption, patients would be systemically exposed to 2.8 mg of F-Ara-A per day. This corresponds to a safety margin of 16. For the subsequent lower concentrations, the safety factor would be 160, 1600, 16000, 160000, 1600000, and 16000000.

The F-Ara-A concentrations discussed above in a)-c) are in the concentration range to inhibit the growth of lymphocytes in vitro. From the above examples, at the most sever case where 40 grams of a 1.0% F-Ara-A topical formulation is delivered per day this treatment would still be 1.6 times more safe than the oncology dose. For most patients with mild to moderate topical autoimmune conditions, the application would be less than 12 grams per day. At 12 grams per day this treatment would be 5.4 times safer than the oncology dose. To improve the therapeutic index, the treatment schedule could be extended and lower concentrations administered thereby increasing the safety margins in the range of 5.4 to 16000000. The F-Ara-A concentrations listed above are estimates based upon an assumed penetration of drug, and the concentration ranges of F-Ara-A that inhibits the growth of monocytes in vitro. (Niitsu et al, 2000) Assuming that intracellular levels of F-Ara-A are sufficient to generate elevated levels of F-Ara-ATP, the above examples of single agent (monotherapy) will be adequate to treat patients with autoimmune diseases.

Another topical formulation would be 0.0002% (0.002 mg/g) F-Ara-A, which is equivalent to approximately 0.5 μM achieved in the skin assuming 7% penetration. According to in vitro data, 0.5 μM is the concentration that causes 50% inhibition of monocytes. (Niitsu et al, 2000) Using the most dose intensive regimen of 40 grams delivered each day, a topical formulation consisting of 0.0002% F-Ara-A and 7% penetration would be considered 8036 times safer than the oncology dose.

To further improve the therapeutic index, the treatment schedule could be prolonged while using reduced concentrations of F-Ara-A thereby increasing the safety margins. Based upon the calculations listed above, and the ability to stabilize the molecule by minimizing excipient interaction, it is concluded that F-Ara-A may be used as a safe topical treatment for mild, moderate and severe autoimmune skin diseases.

3) Topical Formulation for 2-fluoroadenine-9b-D-Arabinofuranoside (F-Ara-A or Fludarabine des-phosphate)

One-Part Formulation—F-Ara-A, like other nucleosides and their analogs, are often stable under specific conditions. Prophetic formulations are given as examples below. A one-part formulation includes F-Ara-A combined with a base topical delivery system, such as a solution, gel, cream, or ointment. The concentration of water varies with each formulation where a solution base may have 25-80 w/w % water; a gel base may have 25-95 w/w % water, a cream base may have 50-80 w/w % water; and an ointment base may have between 1-10 w/w % water. Nucleosides and their analogs are most stable in a pH range of 5 to 9, so all formulations are buffered respectively. Below are four-examples of a one-part formulation using different bases.

| | | |
|---|---|---|
| Component | Function | % w/w |

a) Solution Formulation

| | | |
|---|---|---|
| 2-fluoroadenine-9b-D- | Active | 0.000001-1.0 |
| Arabinofuranoside | | |
| Isopropyl Alcohol | Solvent | 35-55 |
| Propylene Glycol | Solvent | 1-15 |
| Hydroxypropyl Cellulose | Thickening | 0-5 |
| | agent | |
| Phosphoric Acid | Acidifying | pH 5-9 |
| | Agent | |
| Dibasic sodium phosphate | Base | 0.01-1.5 |
| Menthol | Odorant | 0-1 |
| Purified Water | Diluent | 25-80 |

b) Gel Formulation

| | | |
|---|---|---|
| 2-fluoroadenine-9b-D- | Active | 0.000001-1.0 |
| Arabinofuranoside | | |
| Propylene glycol | Solvent | 0.1-10 |
| Methylparaben | Preservative | 0.01-0.1 |
| Propylparaben | Preservative | 0.01-0.1 |
| Edetate Disodium | Chelating agent | 0.01-0.1 |
| Dibasic sodium phosphate | Basic Agent | 0.01-1.5 |
| Carbomer | Gelling Agent | 0.1-2 |
| Phosphoric Acid | Neutralizing | pH 5-9 |
| | Agent | |
| Ethanol | Solvent | 0-75 |
| Purified Water | Solvent | 25-95 |

c) Cream Formulation

| | | |
|---|---|---|
| 2-fluoroadenine-9b-D- | Active | 0.000001-1.0 |
| Arabinofuranoside | | |
| Sorbitol 70% Solution | Humectant | 1-3 |
| Emulsifying Wax | Cream Base | 5-25 |
| Glycerin | Emollient | 0-20 |
| Isopropyl Palmitate | Penetration | 1-10 |
| Benzyl alcohol | Preservative | 0.1-0.5 |
| Edetate disodium | Chelating agent | 0.01-0.55 |
| Dibasic sodium phosphate | Basic Agent | 0.01-0.55 |
| Phosphoric Acid | Neutralizing Agent | pH 5-9 |
| Ceteth 20 | Surfactant | 0.5-5 |
| Mineral Oil | Emollient | 0-55 |
| Purified Water | Solvent | 50-80 |

d) Ointment Formulation

| Excipient | Function | % w/w |
|---|---|---|
| 2-fluoroadenine-9b-D- | Active | 0.000001-1.0 |
| Arabinofuranoside | | |
| Microcrystalline Wax | Ointment base | 0-15 |
| White Petrolatum | Ointment base | 55-99 |
| Tocopherol | Anti-oxidant | 0-0.5 |
| Steareth-2 | Surfactant | 1-10 |
| Propylene Glycol | Solvent | 1-10 |
| Edetate disodium | Chelating agent | 0.001-0.55 |
| Dibasic sodium phosphate | Basic Agent | 0.01-0.55 |
| Phosphoric Acid | Neutralizing Agent | pH 5-9 |
| Purified Water | Solvent | 0-10 |

Two-Part Formulation—Fludara®, a commercial oncology injectable product has a two-year shelf life and it is supplied as a lyophilized solid cake with mannitol and sodium hydroxide. Given the two-year stability of Fludarabine in a lyophilized cake at 2-8° C., a two-part formulation may be necessary for product stability. The two-part product would consist of a F-Ara-A in a lyophilized cake with mannitol and sodium hydroxide in container A (e.g., a syringe) and a topical vehicle in container B (e.g., a syringe). Prior to administration, the F-Ara-A solution and delivery components are mixed thoroughly by, for example, repetitively transferring the components between the syringes. In this way, the product is reconstituted.

A two-part (mix and use) formulation minimizes the degradation that may be possible due to excipient or vehicle interaction since F-Ara-A is exposed to the excipients only briefly before application. The preferred two-part formulation uses a cream base; however, a gel, solution, or ointment base may also be implemented. An example of a two-part cream formulation is listed below.

e) Two-Part Cream Formulation
Part 1 - Drug solution for lyophilization: Container/syringe A

| Component | Function | % w/v |
|---|---|---|
| 2-fluoroadenine-9b-D- | Active | 0.0000026-2.6 |
| Arabinofuranoside | | |
| Mannitol | Bulking Agent | 0.0-8.0 |
| Polyethylene Glycol 4000 | Bulking Agent | 0.0-8.0 |
| Water for Injection | Solvent | QS 100 |

Fill Target = 0.4 mL in a 3-cc Becton Dickinson Sterifill Syringe
Final Drug Amount = 0.05 mg

Part 2 - Cream Base for reconstitution: Container/syringe B

| Component | Function | % w/w |
|---|---|---|
| Sorbitol 70% Solution | Humectant | 1.0-5.0 |
| Emulsifying Wax | Cream Base | 5.0-20.0 |
| Glycerin | Emollient | 0-5.0 |
| Isopropyl Palmitate | Penetration | 2.0-10.0 |
| Benzyl alcohol | Preservative | 0.1-0.5 |
| Dibasic sodium phosphate | Basic Agent | 0.1-0.5 |
| Phosphoric Acid | Neutralizing Agent | pH 5-9 |
| Purified Water | Solvent | QS 100 |

Fill Target = 1 g in a 3-cc female Ultratek Syringe

Final Reconstituted Product

| Component | Function | % w/w |
|---|---|---|
| 2-fluoroadenine-9b-D- | Active | 0.000001-1.0 |
| Arabinofuranoside | | |
| Mannitol | Bulking Agent | 0-21 |
| Polyethylene Glycol 4000 | Bulking Agent | 0-21 |
| Sorbitol 70% Solution | Humectant | 1-5 |
| Emulsifying Wax | Cream Base | 5-20 |
| Glycerin | Emollient | 0-5 |
| Isopropyl Palmitate | Penetration | 2-10 |
| Benzyl alcohol | Preservative | 0.1-0.5 |
| Dibasic sodium phosphate | Basic Agent | 0.01-0.10 |
| Phosphoric Acid | Neutralizing Agent | pH 5-9 |
| Purified Water | Solvent | QS 100 |

Reconstitution: Syringe A and Syringe B are coupled together by means of
integrated Leur Loks.
The product is mixed by first passing the Cream Base into the Drug
Syringe, and then returning the mixture to Syringe B. The product is
mixed back and forth in this manner 30-100 times to completely mix
the drug with the cream. The cream formulation is then dispensed from
the Syringe A for application.

### Example 28

Cytosine 1β-D-Arabinofuranoside (Ara-C)

1) Range of Cytosine 1β-D-Arabinofuranoside (Ara-C) Concentrations in Topical Formulations

To date, the nucleoside Ara-C has not been formulated into a topical delivery system. Several potential solutions to the instability issue are provided as examples below. Experiments conduced in vitro have concluded that Ara-C is effective at cell killing by apoptosis in 2-4 days. The degree of penetration of each drug into the dermis is unknown, so formulations are developed using a combination of empirical and inductive experiments. The concentration range for Ara-C is guided by safety data from clinical studies in which leukemia patients received IV Ara-C. Based upon in vitro and in vivo data, Ara-C monotherapy would be effective, provided that endogenous (intradermal) Ara-C accumulates during therapy. However, the effect may take many days to gain momentum.

The oncology dose is 30 mg/m² every day for 4 days. Based upon the assumption that the average body surface area is 1.8 m², a safe dose of Ara-C is 54 mg every day. For oncology patients, at this dosage, the adverse events were mild to moderate and diminished with treatment, therefore 54 mg/m²/day is referred to as the safe dose that can be administered systemically.

Based upon topical formulations that are similar to the formulations of Ara-C, it is estimated that the systemic absorption of Ara-C will be no more than 7%. Calculations of safety margins have been performed, based upon this assumption and the known toxicity profile of Ara-C when administered IV for oncology indications. Systemic absorption is a concern, because of the potential for serious adverse events, such as lymphopenia and renal toxicity. At oncology doses, these adverse reactions are usually minimized, but the therapeutic index is narrow. Another factor that plays a role in the safety of the formulation is the amount of product administered each day. Approximately 4 grams, 12 grams, and 40 grams of a topical formulation are estimated to cover 10%, 30%, and 100% of the body surface area. Below are some examples of safety margin calculations using Ara-C monotherapy, based upon 7% estimated fractional absorption, and various dose intensities.

2) Concentration Ranges, Treatment Schedules and Safety Margins When Cytosine 1β-D-Arabinofuranoside (Ara-C)is Used as a Monotherapy

a) Apply a topical formulation of Ara-C in the concentration range of about 0.000001 to about 1.0 w/w % to the inflamed areas, with or without an adhesive skin patch, at 100% of the BSA, for a period of 1 day to 6 months. The number of concurrent treatments could be up to as many as 3 treatments per day for a period of up to about 12 weeks. After each treatment the inflamed area may be washed with soap and water and dried before the next application.

Using the highest concentration of 1.0% (10 mg/g), 100% BSA coverage (40 grams) and 7% systemic absorption, patients would be systemically exposed to 28 mg of Ara-C per day. This corresponds to a safety margin of 1.9 or 1.9 times more safe than the oncology treatment (54 mg/day). For the subsequent lower concentrations the safety factor would be 19, 190, 1900, 19000, 190000, and 1900000.

b) Apply a topical formulation of Ara-C in the concentration range of about 0.000001 to about 1.0 w/w % to the inflamed areas, with or without an adhesive skin patch, at 30% of the BSA, for a period of 1 day to 6 months. The number of concurrent treatments could be up to as 3 treatments per day for a period of up to about 12 weeks. After each treatment the inflamed area may be washed with soap and water and dried before the next application.

Using the highest concentration of 1.0% (10 mg/g), 30% BSA coverage (12 grams) and 7% systemic absorption, patients would be systemically exposed to 8.4 mg of Ara-C per day. This corresponds to a safety margin of 6.4. For the subsequent lower concentrations, the safety factor would be 64, 640, 6400, 64000, 640000, and 6400000.

c) Apply a topical formulation of Ara-C in the concentration range of about 0.000001 to about 1.0 w/w % to the inflamed areas, with or without an adhesive skin patch, at 10% of the BSA, for period of 1 day to about 6 months. The number of concurrent treatments could be up to as many as 3 treatments per day for a period of up to about 12 weeks. After each treatment the inflamed area may be washed with soap and water and dried before the next application.

Using the highest concentration of 1.0% (10 mg/g), 10% BSA coverage (4 grams) and 7% systemic absorption, patients would be systemically exposed to 2.8 mg of Ara-C per day. This corresponds to a safety margin of 19. For the subsequent lower concentrations, the safety factor would be 190, 1900, 19000, 190000, 1900000, and 19000000.

The Ara-C concentrations proposed in above in a)-c) are in the concentration range to inhibit the growth of lymphocytes in vitro. From the above examples, at the most sever case where 40 grams of a 1.0% Ara-C topical formulation is delivered per day this treatment would still be 1.9 times more safe than the oncology dose. For most patients with mild to moderate topical autoimmune conditions, the application would be less than 12 grams per day. At 12 grams per day this treatment would be 6.4 times safer than the oncology dose. To improve the therapeutic index, the treatment schedule could be extended and lower concentrations administered thereby increasing the safety margins in the range of 6.4 to 19000000. The Ara-C concentrations listed above are estimates based upon an assumed penetration of drug, and the concentration ranges of Ara-C that inhibits the growth of monocytes in vitro. (Niitsu et al, 2000) Assuming that intracellular levels of Ara-C are sufficient to generate elevated levels of Ara-C, the above examples of single agent (monotherapy) will be adequate to treat patients with autoimmune diseases.

Another topical formulation would be 0.0002% (0.002 mg/g) Ara-C, which is equivalent to approximately 0.5 μM achieved in the skin assuming 7% penetration. According to in vitro data, 0.5 μM is the concentration needed to cause 50% inhibition of monocytes. (Niitsu et al, 2000) Using the most dose intensive regimen of 40 grams delivered each day, a topical formulation consisting of 0.0002% Ara-C and 7% penetration would be considered 9643 times safer than the oncology dose.

To further improve the therapeutic index, the treatment schedule could be prolonged while using reduced concentrations of Ara-C thereby increasing the safety margins. Based upon the calculations listed above, and the ability to stabilize the molecule by minimizing excipient interaction, it is concluded that Ara-C may be used as a safe topical treatment for mild, moderate and severe autoimmune skin diseases. 3) Topical Formulation for Cytosine 1b-D-Arabinofuranoside (Ara-C)

One-Part Formulation—Ara-C, like other nucleosides and their analogs, are often stable under specific conditions. Prophetic formulations are given below as examples. A one-part formulation includes Ara-C combined with a base topical delivery system, such as a solution, gel, cream, or ointment. The concentration of water varies with each formulation where a solution base may have 25-80 w/w % water; a gel base may have 25-95 w/w % water; a cream base may have 50-80 w/w % water; and an ointment base may have between 1-10 w/w % water. Nucleosides and their analogs are most stable in a pH range of 5 to 9, so all formulations are buffered respectively. Below are four-examples of a one-part formulation using different bases.

| | | |
|---|---|---|
| Component | Function | % w/w |

a) Solution Formulation

| | | |
|---|---|---|
| Cytosine 1b-D- | Active | 0.000001-1.0 |
| Arabinofuranoside | | |
| Isopropyl Alcohol | Solvent | 35-55 |
| Propylene Glycol | Solvent | 1-15 |
| Hydroxypropyl Cellulose | Thickening | 0-5 |
| | agent | |
| Phosphoric Acid | Acidifying | pH 5-9 |
| | Agent | |
| Dibasic sodium phosphate | Base | 0.01-1.5 |
| Menthol | Odorant | 0-1 |
| Purified Water | Diluent | 25-80 |

b) Gel Formulation

| | | |
|---|---|---|
| Cytosine 1b-D- | Active | 0.000001-1.0 |
| Arabinofuranoside | | |
| Propylene glycol | Solvent | 0.1-10 |
| Methylparaben | Preservative | 0.01-0.1 |
| Propylparaben | Preservative | 0.01-0.1 |
| Edetate Disodium | Chelating agent | 0.01-0.1 |
| Dibasic sodium phosphate | Basic Agent | 0.01-1.5 |
| Carbomer | Gelling Agent | 0.1-2 |
| Phosphoric Acid | Neutralizing | pH 5-9 |
| | Agent | |
| Ethanol | Solvent | 0-75 |
| Purified Water | Solvent | 25-95 |

c) Cream Formulation

| | | |
|---|---|---|
| Cytosine 1b-D- | Active | 0.000001-1.0 |
| Arabinofuranoside | | |
| Sorbitol 70% Solution | Humectant | 1-3 |
| Emulsifying Wax | Cream Base | 5-25 |
| Glycerin | Emollient | 0-20 |
| Isopropyl Palmitate | Penetration | 1-10 |
| Benzyl alcohol | Preservative | 0.1-0.5 |
| Edetate disodium | Chelating agent | 0.01-0.55 |
| Dibasic sodium phosphate | Basic Agent | 0.01-0.55 |
| Phosphoric Acid | Neutralizing Agent | pH 5-9 |
| Ceteth 20 | Surfactant | 0.5-5 |
| Mineral Oil | Emollient | 0-55 |
| Purified Water | Solvent | 50-80 |

d) Ointment Formulation

| Excipient | Function | % w/w |
|---|---|---|
| Cytosine 1b-D- | Active | 0.000001-1.0 |
| Arabinofuranoside | | |
| Microcrystalline Wax | Ointment base | 0-15 |
| White Petrolatum | Ointment base | 55-99 |
| Tocopherol | Anti-oxidant | 0-0.5 |
| Steareth-2 | Surfactant | 1-10 |
| Propylene Glycol | Solvent | 1-10 |
| Edetate disodium | Chelating agent | 0.001-0.55 |
| Dibasic sodium phosphate | Basic Agent | 0.01-0.55 |
| Phosphoric Acid | Neutralizing Agent | pH 5-9 |
| Purified Water | Solvent | 0-10 |

Two-Part Formulation—Cytosar-U®, a commercial oncology injectable product has a two-year shelf life and it is supplied as a lyophilized solid cake. Given the two-year stability of Ara-C in a lyophilized cake at 20-25° C., a two-part formulation may be necessary for product stability. The two-part product would consist of Ara-C in a lyophilized cake in Container A (e.g., a syringe) and a topical vehicle in Container B (e.g., syringe). Prior to administration, the Ara-C solution and delivery components are mixed thoroughly, for example, by repetitively transferring the components between the syringes. In this way, the product is reconstituted.

A two-part (mix and use) formulation minimizes the degradation that may be possible due to excipient or vehicle interaction since Ara-C is exposed to the excipients only briefly before application. The preferred two-part formulation uses a cream base; however, a gel, solution, or ointment base may also be implemented. An example of a two-part cream formulation is listed below.

e) Two-Part Cream Formulation
Part 1 - Drug solution for lyophilization: Container/Syringe A

| Component | Function | % w/v |
|---|---|---|
| Cytosine 1b-D- | Active | 0.0000026-2.6 |
| Arabinofuranoside | | |
| Mannitol | Bulking Agent | 0.0-8.0 |
| Polyethylene Glycol 4000 | Bulking Agent | 0.0-8.0 |
| Water for Injection | Solvent | QS 100 |

Fill Target = 0.4 mL in a 3-cc Becton Dickinson Sterifill Syringe
Final Drug Amount = 0.05 mg

Part 2 - Cream Base for reconstitution: Container/Syringe B

| Component | Function | % w/w |
|---|---|---|
| Sorbitol 70% Solution | Humectant | 1.0-5.0 |
| Emulsifying Wax | Cream Base | 5.0-20.0 |
| Glycerin | Emollient | 0-5.0 |
| Isopropyl Palmitate | Penetration | 2.0-10.0 |
| Benzyl alcohol | Preservative | 0.1-0.5 |
| Dibasic sodium phosphate | Basic Agent | 0.1-0.5 |
| Phosphoric Acid | Neutralizing Agent | pH 5-9 |
| Purified Water | Solvent | QS 100 |

Fill Target = 1 g in a 3-cc female Ultratek Syringe

Final Reconstituted Product

| Component | Function | % w/w |
|---|---|---|
| Cytosine 1b-D- | Active | 0.000001-1.0 |
| Arabinofuranoside | | |
| Mannitol | Bulking Agent | 0-21 |
| Polyethylene Glycol 4000 | Bulking Agent | 0-21 |
| Sorbitol 70% Solution | Humectant | 1-5 |
| Emulsifying Wax | Cream Base | 5-20 |
| Glycerin | Emollient | 0-5 |
| Isopropyl Palmitate | Penetration | 2-10 |
| Benzyl alcohol | Preservative | 0.1-0.5 |
| Dibasic sodium phosphate | Basic Agent | 0.01-0.10 |
| Phosphoric Acid | Neutralizing Agent | pH 5-9 |
| Purified Water | Solvent | QS 100 |

Reconstitution: Syringe A and Syringe B are coupled together by means of
integrated Leur Loks.
The product is mixed by first passing the Cream Base into the Drug
Syringe, and then returning the mixture to Syringe B. The product
is mixed back and forth in this manner 30-100 times to completely
mix the drug with the cream. The cream formulation
is then dispensed from the Syringe A for application.

### Example 29

Specific formulations that include dCF and/or dAdo and/or CdA, in the amounts described below, can be prepared and used to treat skin disorders. Such compositions will typically have the skin absorption profile as described below. In vitro data presented herein show dCF+dAdo to be toxic to U-937 cells in combination at 0.1 μM and 22 μM, respectively. Additionally, dAdo at 250 μM and CdA at 0.1 μM (Niiitsu et al 2000) were shown to be toxic to U-937 cells on their own Assuming 7% penetration of a dermal formulation, appropriate concentrations are shown in bold.

| | | | Concentration in |
|---|---|---|---|
| Topical | | | the skin assuming |
| Concentration | | | 7% penetration |
| % dCF | ug/mL | uM | uM |
| 0.00001 | 0.1 | 0 | 0.03 |
| 0.00005 | 0.5 | 2 | 0.13 |
| 0.0001 | 1 | 4 | 0.26 |
| 0.001 | 10 | 37 | 2.61 |
| 0.01 | 100 | 373 | 26.09 |
| 0.1 | 1000 | 3728 | 260.93 |
| % dAdo | ug/mL | mM | uM |
| 0.00005 | 0.5 | 0.002 | 0.14 |
| 0.0005 | 5 | 0.02 | 1.39 |
| 0.005 | 50 | 0.20 | 13.93 |
| 0.01 | 100 | 0.40 | 27.86 |
| 0.05 | 500 | 2 | 139.31 |
| 0.1 | 1000 | 4 | 278.62 |
| 0.5 | 5000 | 20 | 1393.09 |
| 1 | 10000 | 40 | 2786.18 |
| 3 | 30000 | 119 | 8358.54 |
| 5 | 50000 | 199 | 13930.90 |
| % CdA | | | |
| 0.000001 | ug/mL | mM | uM |
| 0.00001 | 0.1 | 0.0004 | 0.02 |
| 0.00005 | 0.5 | 0.0018 | 0.12 |
| 0.0001 | 1 | 0.004 | 0.25 |
| 0.001 | 10 | 0.035 | 2.45 |
| 0.01 | 100 | 0.350 | 24.50 |
| 0.1 | 1000 | 3.500 | 245.01 |

The safety factor of 0.00005% dCF+0.01% dAdo in combination is estimated to by 756. For dAdo, no toxicity data is available to correlate to safety but it is readily metabolized to naturally according products, so the risk is assumed to be negligible. The safety factor for 0.00005% CdA is estimated to be 17,142. All safety factors are estimated by covering 30% of body surface area (10.8 grams/day), assuming 7% systemic absorption, and compared to the oncology doses (4 mg/m²/14 days for dCF and 3.6 mg/m²/day for CdA).

Concentrations may be increased or decreased based on the actual systemic exposure or absorption.

The concentrations of the respective compounds have been determined to be toxic and induce apoptosis at levels achieved in the skin with safety factors much greater than the oncology dose. Thus, three preferred embodiments of the invention provide topical formulations including: 1) dCF and dAdo in combination 2) dAdo alone, and 3) CdA alone.

### Example 30

The formulation of any one of Examples 1-28 is administered to a patient previously afflicted with psoriasis, and is currently in remission. The symptoms of the disease may be worse than it was prior to the biological therapy (called rebound or flare). The use of the formulation of any one of Examples 1-28 has several potential implications/benefits. For instance, dAd alone, cladribine alone, dCF alone or dAd+dCF (very low concentrations, concurrent or sequentially applied), F-Ara-A or Ara-C are presumably sufficiently non toxic to allow the patient to apply the product to a greater proportion of the body surface area (e.g., more than 10% which means it could be used in moderate to severe disease). Also, the improved therapeutic index allows the patient to apply it for long periods of time without suffering the cumulative toxicity observed with all other topical therapies (calcipotrienes, retinoids, corticosteroids, PUVA, UVB).

### Example 31

Cytokine Secretion and Caspase-3 Induction in U-937 and Jurkat Cells by iTAP (immunoapoptotic (programmed cell death of selected immunocytes) Topical (e.g., cream, ointment, lotion or gel) Agent (e.g., active pharmaceutical ingredient) for Psoriasis (and other autoimmune skin diseases))

Introduction

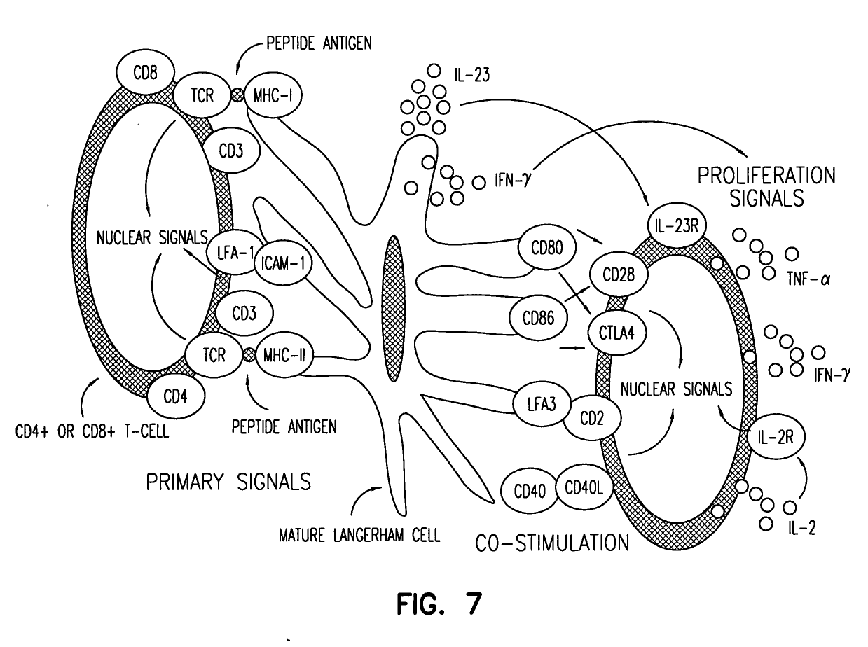

Psoriasis is a Type 1 T-cell (CD4+ and CD8+), immune-mediated disease characterized by hyperproliferative keratinocytes. This produces psoriatic lesions such as erythema and scaling. Psoriatic skin has been associated with the infiltration of antigen presenting cells (APC's or Langerhan) and T-lymphocytes (T-cells). Psoriasis can be triggered by a number of different agents including drugs and bacterial and fungal infections. Activated antigen presenting and T-cells produce abnormal cytokines levels in psoriasis, some of which are IL-2, IL-23, TNF-α, and IFN-γ (FIG. 7).

Nucleoside (2′-deoxyadenosine, dAd) and nucleoside analogs (Cladribine, CdA) are known to induce apoptosis in monocytoid (U-937) and T-lymphocyte (Jurkat) cells. They posses toxicity after they are phosphorylated by a nucleoside kinases (i.e. deoxycytidine kinase) to the triphosphate, namely 2′-deoxyadenosine-5′-triphosphate (dATP) and 2-chloro-2′-deoxyadenosine-5′-triphosphate (CdATP). dAd posses toxicity at much higher levels due to its ability to be catabolized by adenosine deaminase (ADA). CdA is not susceptible to this catabolism due to the halogen at the 2-position, therefore making it more potent. Apoptosis for both compounds in monocytes and lymphocytes is initiated through the inhibition of ribonucleotide reductase (RNR) and stimulation of poly(ADP) ribose polymerase (PARP). This causes a decrease in deoxynucleotide (dNTP) pools and adenosine-5′-triphosphate (ATP) concentrations, respectively, within the cell. This results in the induction of Caspase-3, which causes the fragmentation of DNA. Other halogenated nucleoside analogs are also thought to work by this mechanism such as 2-fluoro-2′-deoxyadenosine (FdA). Cytarabine (Ara-C) has similar toxicity in monocytes and lymphocytes by DNA polymerases inhibition, mainly through chain termination.

In a search for a topical agent that can locally deplete levels of T-cells in the skin, apoptotic compounds were evaluated for their mechanisms of action. The ability that dAd and CdA induce apoptosis by the same mechanisms was examined by cytokine secretion. The experiment was carried out with previously determined apoptotic concentrations of dAd (500 μM), CdA (1 μM), FdA (1 μM) and Ara-C (1 μM) on U-937 and Jurkat cell lines activated by PHA and LPS, respectively. Caspase-3 and cytokine (IL-2, IL-6, TNF, and IFN-γ) levels were determined and compared to controls at 23 and 46 hours. FdA was incorporated to evaluate the effect of the halogen group and Ara-C for its overlapping mechanisms of action.

Methods

U-937 and Jurkat cells were cultured in the presence of 500 μM dAd and 1 μM CdA for 1 and 2 days. At the end of the treatment period, the cells were harvested and Caspase-3 induction was determined using a flow-cytometry-based method to quantify the percentage of cells that induced Caspase-3. The level of apoptosis is expressed as “% FITC shift”. Cytokine secretion (IL-2, IL-6, TNF, and IFN-γ) was measured at the same time by ELISA and is expressed in pg/mL. The cells were exposed to 5 μM camptothecin for 1 and 2 days as a positive control and to “no drug” and “no drug, no activation” as negative controls. Results are presented below.

Results

TNF and IFN-γ are not shown because there was no noticeable difference between the controls and drug treatments. The focus was on IL-6 and IL-2 production because differences were observed based on exposure type. The results are shown in Tables 1 and 2 for IL-6 and IL-2, respectively.

TABLE 1
Caspase-3 induction and cytokine secretion in U-937 cells with
dAd and CdA

| | | | Caspase-3 | Cytokine |
|---|---|---|---|---|
| | | | Induction | Secretion |
| | | | % FITC Shift - | IL-6 pg/ml |
| | Time | | Mean | Mean of 3 |
| Cell Line | (hr) | Drug Treatment | of 3 Replicates | Replicates |
| U-937 | 23 | no drug + LPS | 2 | 8 |
| | | FdA, 1 μM | 67 | 7 |
| | | CdA, 1 μM | 67 | 7 |
| | | dAd, 500 μM | 67 | 4 |
| | | ara-C, 1 μM | 43 | 7 |
| | | Camptothecin, 5 μM | — | 0 |
| U-937 | 46 | no drug, no LPS | 1 | 3 |
| | | no drug + LPS | 2 | 9 |
| | | FdA, 1 μM | 51 | 8 |
| | | CdA, 1 μM | 51 | 7 |
| | | dAd, 500 μM | 43 | 4 |
| | | ara-C, 1 μM | 52 | 7 |
| | | Camptothecin, 5 μM | — | 0 |

TABLE 2
Caspase-3 induction and cytokine secretion in Jurkat cells
with dAd and CdA

| | | | Caspase-3 | Cytokine |
|---|---|---|---|---|
| | | | Induction | Secretion |
| | | | % FITC Shift - | IL-2 pg/ml |
| | | | Mean of 3 | Mean of 3 |
| Cell Line | Time (hr) | Drug Treatment | Replicates | Replicates |
| Jurkat | 23 | no drug | 52 | 50 |
| | | FdA, 1 μM | 75 | 41 |
| | | CdA, 1 μM | 73 | 51 |
| | | dAd, 500 μM | 73 | 17 |
| | | ara-C, 1 μM | 80 | 35 |
| | | Camptothecin, 5 μM | — | 0 |
| Jurkat | 46 | no drug, no PHA | 2 | 0 |
| | | no drug | 85 | 126 |
| | | FdA, 1 μM | 95 | 72 |
| | | CdA, 1 μM | 91 | 105 |
| | | dAd, 500 μM | 92 | 49 |
| | | ara-C, 1 μM | 95 | 65 |
| | | Camptothecin, 5 μM | — | 0 |

Discussion

dAd, CdA, FdA, and Ara-C all induce apoptosis at micromolar concentration range in U-937 and Jurkat cell lines as shown by Caspase-3 induction. This was not determined for Camptothecin, but was shown in a previous experiment to cause apoptosis within a few hours after exposure for both cell lines. The addition of the PHA in the Jurkat cell line produced a baseline level of Caspase-3 without any exposure to drugs.

In U-937 cells, IL-6 is a key cytokine produced by monocytoid cells when activated. Baseline levels can be seen in Table 1 for U-937 cells without drug (3 pg/mL) and for cells activated by LPS (9 pg/mL) after 46 hours. IL-6 levels in the presence of CdA, FdA and Ara-C were 8, 7 and 7 pg/mL, respectively, so there was no significant difference when compared to cells with LPS and no drug. On the contrary, IL-6 levels were inhibited to 4 pg/mL in the presence of dAd.

IL-2 is one of the signals sent when T-cells are activated. This can be seen for Jurkat cells activated by PHA after 46 hours, 126 pg/mL, and much less without simulation by PHA, 0 pg/mL. IL-6 concentrations after Caspase-3 induction were 105, 72, 65, and 49 pg/mL for CdA, FdA, Ara-C and dAd, respectively.

These results suggest that at 46 hours CdA, FdA and Ara-C had little effect on suppressing the production of IL-6 in U-937 cells, while dAd has 55% IL-6 inhibition after Caspase-3 induction when compared to “no drug+LPS.” For the Jurkat T-cells at the same time point, CdA, FdA, Ara-C and dAd all suppress IL-2, but dAd was the most pronounced at 61% inhibition when compared to “no drug+PHA”. Thus, it is a advantageous and unexpected discovery that dAd inhibits cytokine production related to cell activation in U-937 and Jurkat T-cells. Thus, the use of dAd in a topical formulation would not only result in apoptosis of monocytes and lymphocytes in the epidermis, but it would also suppress activation signals that result in activation of the keratinocytes. Monocytes are important because they can differentiate into Langerhan cells that are involved in psoriasis. This is a distinct advantage dAd has over the other nucleoside analogs investigated. The mechanistic differences are not yet understood for the inhibition of these cytokines by dAd.

All publications, patents, and patent documents cited herein are incorporated by reference herein, as though individually incorporated by reference. The invention has been described with reference to various specific and preferred embodiments and techniques. However, it should be understood that many variations and modifications may be made while remaining within the spirit and scope of the invention.

It is appreciated that certain features of the invention, which are, for clarity, described in the context of separate embodiments, may also be provided in combination in a single embodiment. Conversely, various features of the invention which are for brevity, described in the context of a single embodiment, may also be provided separately or in any sub-combination.

1. A method of treating a psoriasis in a mammal, the method comprising topically administering to a mammal in need of such treatment a pharmaceutical composition comprising:
(a) a compound of formula (I): 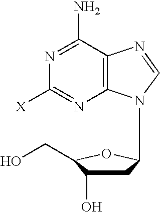
wherein X is F, Cl, Br or I;
(b) a penetration skin enhancer; and
(c) a pharmaceutically acceptable carrier;
in an amount and for a period of time effective to treat the psoriasis.

2. The method of claim 1, wherein X is F.

3. The method of claim 1, wherein X is Cl.

4. The method of claim 1, wherein X is Br.

5. The method of claim 1, wherein X is I.

6. The method of claim 1, wherein the compound of formula (I) is present in about 0.000001 wt. % to about 0.1 wt. % of the pharmaceutical composition.

7. The method of claim 1, wherein the pharmaceutical composition is topically administered, such that the daily dosage of the compound of formula (I) is up to about 6.48 mg.

8. The method of claim 1, wherein the pharmaceutical composition is topically administered, such that systemic absorption of the compound of formula (I) is less than about 7 wt. %.

9. The method of claim 1, wherein the pharmaceutical composition is a gel.

10. The method of claim 1, wherein the pharmaceutical composition is a cream.

11. The method of claim 1, wherein the pharmaceutical composition is a lotion.

12. The method of claim 1, wherein the pharmaceutical composition is a ointment.

13. The method of claim 1, wherein the pharmaceutical composition is administered for up to about 6 months.

14. The method of claim 1, wherein the pharmaceutical composition is administered for up to about 3 months.

15. The method of claim 1, wherein the pharmaceutical composition is administered at least once per day.

16. The method of claim 1, wherein the pharmaceutical composition is administered up to four times per day.

17. The use of a pharmaceutical composition comprising:
(a) a compound of formula (I): 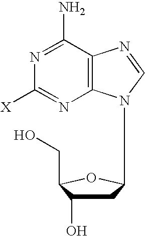
wherein X is F, Cl, Br or I;
(b) a penetration skin enhancer; and
(c) a pharmaceutically acceptable carrier;
for the manufacture of a medicament for treating psoriasis.

18. The use of a composition in claim 17, wherein X is F.

19. The use of a composition in claim 17, wherein X is Cl.

20. The use of a composition in claim 17, wherein X is Br.

21. The use of a composition in claim 17, wherein X is I.

22. The use of a composition in claim 17, wherein the compound of formula (I) is present in about 0.000001 wt. % to about 0.1 wt. % of the pharmaceutical composition.

23. The use of a composition in claim 17, wherein the pharmaceutical composition is topically administered, such that the daily dosage of the compound of formula (I) is up to about 6.48 mg.

24. The use of a composition in claim 17, wherein the pharmaceutical composition is topically administered, such that systemic absorption of the compound of formula (I) is less than about 7 wt. %.

25. The use of a composition in claim 17, wherein the pharmaceutical composition is a gel.

26. The use of a composition in claim 17, wherein the pharmaceutical composition is a cream.

27. The use of a composition in claim 17, wherein the pharmaceutical composition is a lotion.

28. The use of a composition in claim 17, wherein the pharmaceutical composition is a ointment.

29. The use of a composition in claim 17, wherein the pharmaceutical composition is administered for up to about 6 months.

30. The use of a composition in claim 17, wherein the pharmaceutical composition is administered for up to about 3 months.

31. The use of a composition in claim 17, wherein the pharmaceutical composition is administered at least once per day.

32. The use of a composition in claim 17, wherein the pharmaceutical composition is administered up to four times per day.

33. A kit comprising:
(a) a first container comprising a compound of formula (I): 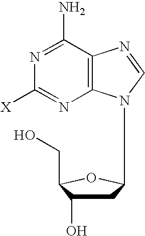
wherein X is F, Cl, Br or I; and
(b) a second container comprising a pharmaceutically acceptable carrier.

34. The kit of claim 33, wherein the first container is a syringe.

35. The kit of claim 33, wherein the second container is a syringe.

36. The kit of claim 33, wherein the first container is a syringe, the second container is a syringe, and the two syringes are adapted to reversibly interconnect in fluid tight engagement with each other.

37. A syringe system that comprises:
a first syringe having a female fitting, the first syringe comprising a first syringe barrel having an inner surface and an open proximal end;
a first syringe plunger having a first stopper tip in slidable communication with the inner surface of the first syringe barrel via the open proximal end, the first stopper tip configured for fluid-tight engagement with a first composition;
a second syringe having a male fitting, the second syringe comprising a second syringe barrel having an inner surface and an open proximal end; and
a second syringe plunger having a second stopper tip in slidable communication with the inner surface of the second syringe barrel via the open proximal end, the second stopper tip configured for fluid-tight engagement with a second composition;
wherein the female fitting is sized to receive and configured to interlock with the male fitting for fluid-tight engagement between the first and the second syringes;
the first syringe comprising a compound of formula (I): 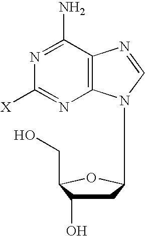
wherein X is F, Cl, Br or I; and the second syringe comprising a pharmaceutically acceptable carrier.

38. The syringe system of claim 37, wherein the female fitting is sized to receive and configured to interlock with the male fitting by a locking ring.

39. The syringe system of claim 37, wherein the locking ring is rotatably coupled with the male fitting and the locking ring is threadingly coupled with one or more projections disposed on an outer surface of the female fitting.

40. The syringe system of claim 37, wherein either or both the female fitting and the male fitting are configured to detachably connect to a discharge assembly.

41. The syringe system of claim 37, wherein a secondary stopper tip is disposed between a primary stopper tip and the proximal end of either or both the first and the second syringe barrels.

42. The syringe system of claim 37, further comprising an outwardly projecting flange near the proximal end of either or both the first syringe and the second syringe.

43. The syringe system of claim 37, wherein each syringe barrel independently has a volume from about 0.01 cc to about 100 cc.

44. The syringe system of claim 37, wherein each syringe barrel independently has a volume from about 0.5 cc to about 10 cc.
